SEQUENCE LISTING (1 sequence)

SEQ ID NO: 1 - DNA, 56 bp, Artificial sequence
  synthetic sequence
taatacgactcactatagagaaggggaggcaaaactggttttagagctagaaatag

(19) United States
(12) Patent Application Publication
(10) Pub. No.: US 2020/0223826 A1
(43) Pub. Date: Jul. 16, 2020

(54) Glucose-6-Phosphate Dehydrogenase (G6PD)-Modulating Agents And Methods Of Treating G6PD Deficiency

(71) Applicant: The Board of Trustees of the Leland Stanford Junior University, Stanford, CA (US)

(72) Inventors: Sunhee Hwang, Stanford, CA (US); DARIA MOCHLY-ROSEN, Menlo Park, CA (US); CHE-HONG CHEN, Fremont, CA (US); Andrew Goodwin Raub, Stanford, CA (US)

(21) Appl. No.: 16/632,863

(22) PCT Filed: Jul. 24, 2018
(86) PCT No.: PCT/US18/43537

§ 371 Date: Jan. 21, 2020

Related U.S. Application Data
(60) Provisional application No. 62/536,925, filed on Jul. 25, 2017.

(51) Int. Cl. C07D 403/10; C07D 407/10; C07D 407/12; C07D 403/12; C07C 215/32; C07C 225/16; C07D 401/12; C07D 409/12; C07C 211/27; C07C 211/42; C07D 487/04; C07D 471/04; C07D 471/06

(52) U.S. Cl. CPC C07D 403/10; C07D 407/10; C07D 407/12; C07D 403/12; C07C 215/32; C07C 225/16; C07D 471/06; C07D 409/12; C07C 211/27; C07C 211/42; C07D 487/04; C07D 471/04; C07D 401/12

(57) ABSTRACT

Aspects of the present disclosure include G6PD-modulating agents and methods for modulating a glucose-6-phosphate dehydrogenase (G6PD) in a sample using such agents. A G6PD-modulating agent can be dimeric and include two terminal carbocyclic or heterocyclic groups connected via a linker. In some instances, the agent includes a diamino-containing linker. In certain cases, the agent includes two amino substituents. Also provided are methods for treating a subject for a G6PD deficiency-associated condition, that include administering to a subject an effective amount of a G6PD-modulating agent to selectively activate a mutant G6PD and treat the subject. Kits and compositions for practicing the subject methods are also provided.

### CROSS-REFERENCE

This application claims the benefit of U.S. Provisional Patent Application No. 62/536,925, filed Jul. 25, 2017, which application is incorporated herein by reference in its entirety.

### GOVERNMENT RIGHTS

This invention was made with Government support under contract HD084422 and TR001085 awarded by the National Institutes of Health. The Government has certain rights in the invention.

### INTRODUCTION

The cells in our body have several mechanisms to counteract oxidative stress by producing antioxidants. One of the natural sources of antioxidants occurs through the activity of an enzyme known as glucose-6-phosphate dehydrogenase (G6PD). G6PD catalyzes the first and rate-limiting step of the pentose phosphate pathway, in which reduced NADPH (nicotinamide adenine dinucleotide phosphate) is generated. NADPH is in turn used to maintain the supply of reduced glutathione (GSH), which plays a critical role in regulating antioxidant balance and thus protecting cells from oxidative damage. Particularly, erythrocytes, which lack mitochondria, have no other means of generating NADPH and rely solely on G6PD for the generation of antioxidants.

Glucose-6-phosphate dehydrogenase (G6PD) deficiency is the second most common human genetic disorder, caused by over 160 different point mutations in G6PD. These mutations may disturb the local structural integrity, which leads to complete or partial loss of the enzyme activity and/or stability and thus disrupts the physiological antioxidant balance with significant decreases in NADPH and GSH levels and thus increases the vulnerability to oxidative stress in cells. Lacking protection against oxidative stress, G6PD-deficient individuals are highly susceptible to hemolytic anemia, neonatal jaundice, and kernicterus (bilirubin-induced brain damage), if left untreated. Currently, despite such outcomes, no medications are available to treat G6PD deficiency other than avoiding oxidative stressors and/or palliative care; thus, compounds and methods of treatment that can correct G6PD deficiency are of great interest.

### SUMMARY

Aspects of the present disclosure include G6PD-modulating agents and methods for modulating a glucose-6-phosphate dehydrogenase (G6PD) in a sample using such agents. A G6PD-modulating agent can be dimeric and include two terminal carbocyclic or heterocyclic groups connected via a linker. In some instances, the agent includes a diamino-containing linker. In certain cases, the agent includes two amino substituents. Also provided are methods for treating a subject for a G6PD deficiency-associated condition, that include administering to a subject an effective amount of a G6PD-modulating agent to selectively activate a mutant G6PD and treat the subject. Kits and compositions for practicing the subject methods are also provided.

### BRIEF DESCRIPTION OF THE FIGURES

FIG. 1A to FIG. 1J together show that Canton G6PD (R459L) variant is biochemically different from WT G6PD. FIG. 1A depicts an enzymatic scheme of G6PD activity. FIG. 1B shows a linear map of G6PD domain structure with common variants of interest indicated. FIG. 1C shows a graph of catalytic activity of recombinant WT G6PD and Canton G6PD enzymes with kinetic parameters (n=5, ***P<0.001). FIG. 1D shows a graph of thermostability of WT G6PD and Canton G6PD enzyme (n=3, P=0.002). FIG. 1E illustrates G6PD protein levels and residual G6PD activity (normalized to NT (no treatment) of each enzyme) after incubation with chymotrypsin for 1 hour (n=3, **P=0.0046, *P=0.0237). FIG. 1F shows a graph indicating protein stability assessment with cycloheximide treatment (50 μg/mL), blocking de novo protein biosynthesis, in lymphocytes derived from corresponding subjects (n=3, *P=0.0255). Protein levels were normalized to the level of each enzyme at 0 hr (no treatment). FIG. 1G shows a graph indicating G6PD activity was lower in cell lysates with Canton variant (n=4, ****P<0.0001). FIG. 1H, FIG. 1I and FIG. 1J show graphs of data that indicate lymphocytes with Canton variant generated less GSH and more reactive oxygen species (ROS) and were less viable (n=4, **P<0.008; n=3, *P=0.0421; n=4, *# P=0.0061). Error bars represent mean±SEM. NT: no treatment; Chy: chymotrypsin.

FIG. 2A to FIG. 2D together illustrate that Canton mutation (R459L) loses essential inter-helical interactions. FIG. 2A shows a structural overlay of WT G6PD (green) and Canton variant (orange). Structural NADP⁺ is shown as spheres, and arrows and circles indicate G6P and catalytic NADP⁺-binding sites (G6P and catalytic NADP⁺ were not observed in our structures). FIG. 2B show the inter-helical interactions through R459 on the an helix in WT G6PD and side chains of D181 and N185 on the adjacent helix (αe) (Left panel). Canton mutation loses such interactions, leading to displacements of the helix (αe) and a loop containing K171, P172, F173, G174, and R175 that precedes the helix (Right panel). FIG. 2C shows data indicating mutation of R459-interacting residues on the αe helix showed Canton mutation-like activity and thermostability (n=3, ***P<0.001) and FIG. 2D shows data indicating susceptibility to chymotrypsin treatment (n=3, ***P=0.0006, *P=0.023). Error bars represent mean±SEM. NT: no treatment; Chy: chymotrypsin.

FIG. 3A to FIG. 3K illustrates that exemplary compound AG1 (activator of G6PD) induces biochemical changes in the Canton variant. FIG. 3A shows a graph indicating AG1 increased the activity of Canton G6PD enzyme by 70%. FIG. 3B shows a graph of AG1 activation with EC₅₀ of ˜3 μM (n=5, ***P=0.0002). AG1 changed kinetic parameters of Canton G6PD (see Table 5 in Experimental section below). FIG. 3C indicates AG1 promoted dimerization of Canton G6PD in a dose-dependent manner (n=3). FIG. 3D shows that AG1 reduced proteolytic susceptibility of Canton G6PD (n=3, *P=0.0177). The protein level was normalized to the level of the enzyme in the no-treatment (NT) condition. FIG. 3E shows AG1 mildly increased protein stability in lymphocytes carrying the Canton variant in cycloheximide-chase assay (n=3). Protein levels were normalized to the level of each enzyme at time 0. FIG. 3F, FIG. 3G and FIG. 3H show data indicating AG1 increased G6PD activity in cell lysates with the Canton variant, mildly enhanced a GSH level and reduced a ROS level in culture (n=4, **P=0.0031). FIG. 3I shows data indicating AG1 slightly increased viability of lymphocytes carrying the Canton variant (n=4). FIG. 3J shows data indicating AG1 also activated other major G6PD variants, including A- (V68M, N126D), Mediterranean (S188F), and Kaiping (R463H) variants, respectively (n=4, **P<0.01, *P=0.011). FIG. 3K shows data indicating AG1 promoted dimerization of other major G6PD variants (n=3). 100 μM of AG1 was used for in vitro assays and 1 μM of AG1 was used for cell-based assays. 5% DMSO was used as vehicle (Veh). Cells were subjected to serum starvation for 48 hours. Error bars represent mean±SEM. MW: molecular weight; NT: no treatment.

FIG. 4A to FIG. 4J illustrate that exemplary compound AG1 attenuates ROS-induced pericardial edema in a G6PD-dependent manner. FIG. 4A shows images of zebrafish embryos that were treated at 24 hpf with 1 μM AG1 with and without chloroquine (CQ; 50 μg/ml) and scored at 32 hpf. Representative phenotypic images are provided on the left. Embryo orientation is lateral view, anterior left. Scale bar: 300 μm. FIG. 4B shows a graph of ROS levels in individual WT embryos from three independent clutches. Embryos were treated at 24 hpf for 5 hours before ROS measurement. Error bars represent mean±SD. (Kruskal-Wallis multiple comparison test, adjusted P value using Dunn's test: ***P<0.001, ns=not statistically significant, P>0.99). FIG. 4C shows graphs of G6PD activity and NADPH levels that were measured with lysates of pooled embryos. Error bars represent mean±SD of the replicate measurements (***P<0.001). FIG. 4D shows images of zebrafish embryos that were injected with either sgRNA targeting Exon 10 of g6pd (Guide Alone) or sgRNA+Cas9 protein (Guide+Cas9) to generate G6PD F0 crispants. Representative phenotypic images are provided on the left panel. Treatment conditions are the same as described for FIG. 4A.

FIG. 4E shows graph of ROS levels in individual embryos with sgRNA or sgRNA+Cas9 protein injection. Treatment conditions and the statistics are the same as described for FIG. 4B (*P=0.0267, **P<0.01, ****P<0.0001, ns=not statistically significant). FIG. 4F shows graphs of G6PD activity and NADPH levels that were measured with lysates of pooled embryos. Error bars represent mean±SD of the replicate measurements (*P<0.05). CQ: chloroquine; Veh: vehicle. FIG. 4G shows chloroquine-induced hemolysis of human erythrocytes was rescued by AG1 (1 μM) treatment (n=3, A-C represents three independent samples). FIG. 4H shows Whether AG1 improves storage of erythrocytes at refrigerated temperature was examined by monitoring hemolysis for 28 days (n=9, *P<0.05). Phenotypic images of hemolysis were provided. FIG. 4I shows protein leakage from erythrocytes was measured to examine membrane damage of erythrocytes (n=9, *P<0.05). FIG. 4J shows G6PD activity in human hemolysate was measured in the presence of AG1 (n=15, *P<0.05). Error bars represent mean±SD (assays in zebrafish) or SEM (assays in erythrocytes) of the replicate measurements. CQ: chloroquine; Veh: vehicle; KO: knockout.

FIG. 5A to FIG. 5H illustrates that AG1 reduces hemolysis upon exposure to oxidative stressors. FIG. 5A shows AG1 reduced the extent of hemolysis of 5% erythrocyte suspension treated with either 1 mM chloroquine (CQ) for 4 hours under light or 1 mM diamide for 4 hours at 37° C. (n=7 independent blood samples, *P=0.0372, **P=0.0019, ****P<0.0001). FIG. 5B to FIG. 5D show AG1 significantly increased GSH levels and reduced ROS levels when 5% erythrocyte suspension was exposed to either 1 mM chloroquine or diamide for 3 hours at 37° C. (n=1, *P<0.05, **P<0.01, ****P<0.0001), which was consistent with increased G6PD activity (n=9 for chloroquine-treated sample assay and n=11 for diamide-treated sample assay, *P<0.05, **P<0.01, ****P<0.0001). FIG. 5E shows band 3 protein was clustered (cBand3) when 5% erythrocyte suspension was treated with chloroquine, which was alleviated by AG1 treatment. Each lane represents one individual sample. FIG. 5F to FIG. 5H show AG1 (1 μM) improved storage of erythrocytes (5% suspension) at refrigerated temperature by reducing hemolysis and concomitant protein leakage for 28 days (n=13 independent blood samples, *P<0.05), which corresponded with increased G6PD activity (n=4, *P=0.0323, ***P=0.0003). Each sample was re-treated with AG1 every week. Representative hemolysis phenotypic images are provided. Error bars represent mean±SD. NT: no treatment; CQ: chloroquine; cBand3: clustered band 3 protein.

FIG. 6A to FIG. 6E illustrates some residual disorder is found in G6P- and catalytic NADP⁺-binding sites of Canton G6PD as compared with WT G6PD. FIG. 6A illustrates that the structural NADP⁺-binding site is well conserved in the structures of WT G6PD and Canton G6PD, shown in green and orange, respectively. Side chains of residues involved in binding to NADP⁺ are illustrated by sticks. Orientation of side chains of essential residues (circled) around (FIG. 6B) G6P- and (FIG. 6C) catalytic NADP⁺-binding sites. FIG. 6D shows a graph illustrating the catalytic activity of K171A and P172G mutant enzymes (n=4, ***P=0.0001). FIG. 6E shows a graph of thermal inactivation curves of WT G6PD, Canton G6PD and mutant enzymes of R459-interacting residues, D181 and N185. T_{1/2} values are summarized in FIG. 1D and FIG. 2C.

FIG. 7A illustrates an expanded view of the X-ray crystal structure of the Canton variant (R459L) of G6PD focused at the G6PD dimer interface and a proposed binding site of G6PD-modulating compounds. The cofactor NADP⁺ (also referred to as structural NADP⁺) is shown as black sticks. FIG. 7B illustrates a expanded view of structure the X-ray crystal structure of the Canton variant (R459L) of G6PD focused at the G6PD dimer interface with an exemplary compound AR3-069 (cyan) docked at the proposed binding site near structural NADP⁺ (black). The docked structure of compound AR3-069 (with residue 509 of the G6PD removed) supports the putative receptor site.

FIG. 8 shows the structure of an exemplary G6PD-modulating compound AR3-069 including measurements of approximate distances between pharmacophoric elements of interest.

FIG. 9A illustrates two views of opposite faces of a space filing representation of the X-ray crystal structure of the Canton variant (R459L) of G6PD. Residues were mutated on either side of the dimer interface. The top panel illustrates various mutations (red, blue) located at the compound binding site. The top panel illustrates the location of various mutations (green, yellow and cyan) at the opposite face of the G6PD enzyme. FIG. 9B shows a graph indicating the effect of various point mutations of the Canton variant of G6PD have on the G6PD-activity of exemplary compound AR3-069. The colored bars of the graph correspond to the mutations depicted in the structure of FIG. 3A. The Residues only at the putative binding site affect binding of the subject compounds whereas there was no effect on AC₅₀ (also EC₅₀) on the other side.

### DEFINITIONS

Before describing exemplary embodiments in greater detail, the following definitions are set forth to illustrate and define the meaning and scope of the terms used in the description. Any undefined terms have their art recognized meanings.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Where compounds described herein contain one or more chiral centers and/or double-bond isomers (i.e., geometric isomers), enantiomers or diastereomers, all possible enantiomers and stereoisomers of the compounds including the stereoisomerically pure form (e.g., geometrically pure, enantiomerically pure or diastereomerically pure) and enantiomeric and stereoisomeric mixtures are included in the description of the compounds herein. Enantiomeric and stereoisomeric mixtures can be resolved into their component enantiomers or stereoisomers using separation techniques or chiral synthesis techniques well known to the skilled artisan. The compounds can also exist in several tautomeric forms including the enol form, the keto form and mixtures thereof. Accordingly, the chemical structures depicted herein encompass all possible tautomeric forms of the illustrated compounds. The compounds described also include isotopically labeled compounds where one or more atoms have an atomic mass different from the atomic mass conventionally found in nature. Examples of isotopes that can be incorporated into the compounds disclosed herein include, but are not limited to, ²H, ³H, ¹¹C, ¹³C, ¹⁴C, ¹⁵N, ¹⁸O, ¹⁷O, etc. Compounds can exist in unsolvated forms as well as solvated forms, including hydrated forms. In general, compounds can be hydrated or solvated. Certain compounds can exist in multiple crystalline or amorphous forms. In general, all physical forms are equivalent for the uses contemplated herein and are intended to be within the scope of the present disclosure.

Unless indicated otherwise, the terms “linker”, “linking group”, “linkage” and “tether” refers to a linking moiety that connects two groups and has a backbone of 100 atoms or less in length. A linker or linkage may be a covalent bond that connects two groups or a chain of between 1 and 100 atoms in length, for example of 1, 2, 3, 4, 5, 6, 8, 10, 12, 14, 16, 18, 20, 30, 40 or 50 atoms (e.g., C, O, N and S atoms) in length, where the linker may be linear, branched, cyclic or a single atom. In certain cases, one, two, three, four or five or more carbon atoms of a linker backbone may be optionally substituted with a sulfur, nitrogen or oxygen heteroatom. The bonds between backbone atoms may be saturated or unsaturated, usually not more than one, two, or three unsaturated bonds will be present in a linker backbone. The linker may include one or more substituent groups, for example with an alkyl, aryl or alkenyl group. A linker may include, without limitations, poly(ethylene glycol) unit(s) (e.g., —(CH₂—CH₂—O)—); ethers, thioethers, amines, alkyls (e.g., (C₁-C₁₂)alkyl), which may be straight or branched, e.g., methyl, ethyl, n-propyl, 1-methylethyl (isopropyl), n-butyl, n-pentyl, 1,1-dimethylethyl (t-butyl), and the like. The linker backbone may include a cyclic group, for example, an aryl, a heterocycle or a cycloalkyl group, where 2 or more atoms, e.g., 2, 3 or 4 atoms, of the cyclic group are included in the backbone. A linker may be cleavable or non-cleavable.

“Alkyl” refers to monovalent saturated aliphatic hydrocarbyl groups having from 1 to 10 carbon atoms and such as 1 to 6 carbon atoms, or 1 to 5, or 1 to 4, or 1 to 3 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

The term “substituted alkyl” refers to an alkyl group as defined herein wherein one or more carbon atoms in the alkyl chain have been optionally replaced with a heteroatom such as —O—, —N—, —S—, —S(O)ₙ— (where n is 0 to 2), —NR— (where R is hydrogen or alkyl) and having from 1 to 5 substituents selected from the group consisting of alkoxy, substituted alkoxy, cycloalkyl, substituted cycloalkyl, cycloalkenyl, substituted cycloalkenyl, acyl, acylamino, acyloxy, amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-aryl, —SO-heteroaryl, —SO₂-alkyl, —SO₂-aryl, —SO₂-heteroaryl, and —NR^{a}R^{b}, wherein R′ and R″ may be the same or different and are chosen from hydrogen, optionally substituted alkyl, cycloalkyl, alkenyl, cycloalkenyl, alkynyl, aryl, heteroaryl and heterocyclic.

“Alkylene” refers to divalent aliphatic hydrocarbyl groups preferably having from 1 to 6 and more preferably 1 to 3 carbon atoms that are either straight-chained or branched, and which are optionally interrupted with one or more groups selected from —O—, —NR¹⁰—, —NR¹⁰C(O)—, —C(O)NR¹⁰- and the like. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Substituted alkylene” refers to an alkylene group having from 1 to 3 hydrogens replaced with substituents as described for carbons in the definition of “substituted” below.

The term “alkane” refers to alkyl group and alkylene group, as defined herein.

The term “alkylaminoalkyl”, “alkylaminoalkenyl” and “alkylaminoalkynyl” refers to the groups R′NHR″— where R′ is alkyl group as defined herein and R″ is alkylene, alkenylene or alkynylene group as defined herein.

The term “alkaryl” or “aralkyl” refers to the groups -alkylene-aryl and -substituted alkylene-aryl where alkylene, substituted alkylene and aryl are defined herein.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like. The term “alkoxy” also refers to the groups alkenyl-O—, cycloalkyl-O—, cycloalkenyl-O—, and alkynyl-O—, where alkenyl, cycloalkyl, cycloalkenyl, and alkynyl are as defined herein.

The term “substituted alkoxy” refers to the groups substituted alkyl-O—, substituted alkenyl-O—, substituted cycloalkyl-O—, substituted cycloalkenyl-O—, and substituted alkynyl-O— where substituted alkyl, substituted alkenyl, substituted cycloalkyl, substituted cycloalkenyl and substituted alkynyl are as defined herein.

The term “alkoxyamino” refers to the group —NH-alkoxy, wherein alkoxy is defined herein.

The term “haloalkoxy” refers to the groups alkyl-O— wherein one or more hydrogen atoms on the alkyl group have been substituted with a halo group and include, by way of examples, groups such as trifluoromethoxy, and the like.

The term “haloalkyl” refers to a substituted alkyl group as described above, wherein one or more hydrogen atoms on the alkyl group have been substituted with a halo group. Examples of such groups include, without limitation, fluoroalkyl groups, such as trifluoromethyl, difluoromethyl, trifluoroethyl and the like.

The term “alkylalkoxy” refers to the groups -alkylene-O-alkyl, alkylene-O-substituted alkyl, substituted alkylene-O-alkyl, and substituted alkylene-O-substituted alkyl wherein alkyl, substituted alkyl, alkylene and substituted alkylene are as defined herein.

The term “alkylthioalkoxy” refers to the group -alkylene-S-alkyl, alkylene-S-substituted alkyl, substituted alkylene-S-alkyl and substituted alkylene-S-substituted alkyl wherein alkyl, substituted alkyl, alkylene and substituted alkylene are as defined herein.

“Alkenyl” refers to straight chain or branched hydrocarbyl groups having from 2 to 6 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

The term “substituted alkenyl” refers to an alkenyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

“Alkynyl” refers to straight or branched monovalent hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

The term “substituted alkynyl” refers to an alkynyl group as defined herein having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl, and —SO₂-heteroaryl.

“Alkynyloxy” refers to the group —O-alkynyl, wherein alkynyl is as defined herein. Alkynyloxy includes, by way of example, ethynyloxy, propynyloxy, and the like.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, cycloalkenyl-C(O)—, substituted cycloalkenyl-C(O)—, aryl-C(O)—, substituted aryl-C(O)—, heteroaryl-C(O)—, substituted heteroaryl-C(O)—, heterocyclyl-C(O)—, and substituted heterocyclyl-C(O)—, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. For example, acyl includes the “acetyl” group CH₃C(O)—

“Acylamino” refers to the groups —NR²⁰C(O)alkyl, —NR²⁰C(O)substituted alkyl, N R²⁰C(O)cycloalkyl, —NR²⁰C(O)substituted cycloalkyl, —NR²⁰C(O)cycloalkenyl, —NR²⁰C(O)substituted cycloalkenyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonyl” or the term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aminocarbonylamino” refers to the group —NR²¹C(O)NR²²R²³ where R²¹, R²², and R²³ are independently selected from hydrogen, alkyl, aryl or cycloalkyl, or where two R groups are joined to form a heterocyclyl group.

The term “alkoxycarbonylamino” refers to the group —NRC(O)OR where each R is independently hydrogen, alkyl, substituted alkyl, aryl, heteroaryl, or heterocyclyl wherein alkyl, substituted alkyl, aryl, heteroaryl, and heterocyclyl are as defined herein.

The term “acyloxy” refers to the groups alkyl-C(O)O—, substituted alkyl-C(O)O—, cycloalkyl-C(O)O—, substituted cycloalkyl-C(O)O—, aryl-C(O)O—, heteroaryl-C(O)O—, and heterocyclyl-C(O)O— wherein alkyl, substituted alkyl, cycloalkyl, substituted cycloalkyl, aryl, heteroaryl, and heterocyclyl are as defined herein.

“Aminosulfonyl” refers to the group —SO₂NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, substituted heterocyclic and where R²¹ and R²² are optionally joined together with the nitrogen bound thereto to form a heterocyclic or substituted heterocyclic group and alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic and substituted heterocyclic are as defined herein.

“Sulfonylamino” refers to the group —NR²¹SO₂R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the atoms bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Aryl” or “Ar” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Aryloxy” refers to the group —O-aryl, wherein aryl is as defined herein, including, by way of example, phenoxy, naphthoxy, and the like, including optionally substituted aryl groups as also defined herein.

Unless indicated otherwise, “amino” refers to the group —NH₂.

The term “substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

The term “azido” refers to the group —N₃.

“Carboxyl,” “carboxy” or “carboxylate” refers to —CO₂H or salts thereof.

“Carboxyl ester” or “carboxy ester” or the terms “carboxyalkyl” or “carboxylalkyl” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-cycloalkyl, —C(O)O-substituted cycloalkyl, —C(O)O-cycloalkenyl, —C(O)O-substituted cycloalkenyl, —C(O)O-heteroaryl, —C(O)O-substituted heteroaryl, —C(O)O-heterocyclic, and —C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“(Carboxyl ester)oxy” or “carbonate” refers to the groups —O—C(O)O— alkyl, —O—C(O)O-substituted alkyl, —O—C(O)O-alkenyl, —O—C(O)O-substituted alkenyl, —O—C(O)O— alkynyl, —O—C(O)O-substituted alkynyl, —O—C(O)O-aryl, —O—C(O)O-substituted aryl, —O—C(O)O— cycloalkyl, —O—C(O)O-substituted cycloalkyl, —O—C(O)O-cycloalkenyl, —O—C(O)O-substituted cycloalkenyl, —O—C(O)O-heteroaryl, —O—C(O)O-substituted heteroaryl, —O—C(O)O-heterocyclic, and —O—C(O)O-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “nitrile” refers to the group —CN.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantanyl, and the like.

The term “substituted cycloalkyl” refers to cycloalkyl groups having from 1 to 5 substituents, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

“Cycloalkenyl” refers to non-aromatic cyclic alkyl groups of from 3 to 10 carbon atoms having single or multiple rings and having at least one double bond and preferably from 1 to 2 double bonds.

The term “substituted cycloalkenyl” refers to cycloalkenyl groups having from 1 to 5 substituents, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, keto, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO-alkyl, —SO-substituted alkyl, —SO-aryl, —SO— heteroaryl, —SO₂-alkyl, —SO₂-substituted alkyl, —SO₂-aryl and —SO₂-heteroaryl.

“Cycloalkynyl” refers to non-aromatic cycloalkyl groups of from 5 to 10 carbon atoms having single or multiple rings and having at least one triple bond.

“Cycloalkoxy” refers to —O-cycloalkyl.

“Cycloalkenyloxy” refers to —O-cycloalkenyl.

“Halo” or “halogen” refers to fluoro, chloro, bromo, and iodo.

“Hydroxy” or “hydroxyl” refers to the group —OH.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuran, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxylalkyl, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, —SO-alkyl, —SO-substituted alkyl, —SO-aryl, —SO— heteroaryl, —SO₂-alkyl, —SO₂-substituted alkyl, —SO₂-aryl and —SO₂-heteroaryl, and trihalomethyl.

The term “heteroaralkyl” refers to the groups -alkylene-heteroaryl where alkylene and heteroaryl are defined herein. This term includes, by way of example, pyridylmethyl, pyridylethyl, indolylmethyl, and the like.

“Heteroaryloxy” refers to —O-heteroaryl.

“Heterocycle,” “heterocyclic,” “heterocycloalkyl,” and “heterocyclyl” refer to a saturated or unsaturated group having a single ring or multiple condensed rings, including fused bridged and spiro ring systems, and having from 3 to 20 ring atoms, including 1 to 10 hetero atoms. These ring atoms are selected from the group consisting of nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for the N-oxide, —S(O)—, or —SO₂— moieties.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, tetrahydrofuranyl, and the like.

Unless otherwise constrained by the definition for the heterocyclic substituent, such heterocyclic groups can be optionally substituted with 1 to 5, or from 1 to 3 substituents, selected from alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, oxyaminoacyl, azido, cyano, halogen, hydroxyl, oxo, thioketo, carboxyl, carboxylalkyl, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, —SO— alkyl, —SO-substituted alkyl, —SO-aryl, —SO-heteroaryl, —SO₂-alkyl, —SO₂-substituted alkyl, —SO₂-aryl, —SO₂-heteroaryl, and fused heterocycle.

“Heterocyclyloxy” refers to the group —O-heterocyclyl.

The term “heterocyclylthio” refers to the group heterocyclic-S—.

The term “heterocyclene” refers to the diradical group formed from a heterocycle, as defined herein.

The term “hydroxyamino” refers to the group —NHOH.

“Nitro” refers to the group —NO₂.

“Oxo” refers to the atom (═O).

“Sulfonyl” refers to the group SO₂-alkyl, SO₂-substituted alkyl, SO₂-alkenyl, SO₂-substituted alkenyl, SO₂-cycloalkyl, SO₂-substituted cycloalkyl, SO₂-cycloalkenyl, SO₂-substituted cylcoalkenyl, SO₂-aryl, SO₂-substituted aryl, SO₂-heteroaryl, SO₂-substituted heteroaryl, SO₂-heterocyclic, and SO₂-substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein. Sulfonyl includes, by way of example, methyl-SO₂—, phenyl-SO₂—, and 4-methylphenyl-SO₂—.

“Sulfonyloxy” refers to the group —OSO₂-alkyl, OSO₂-substituted alkyl, OSO₂-alkenyl, OSO₂-substituted alkenyl, OSO₂-cycloalkyl, OSO₂-substituted cycloalkyl, OSO₂-cycloalkenyl, OSO₂-substituted cylcoalkenyl, OSO₂-aryl, OSO₂-substituted aryl, OSO₂-heteroaryl, OSO₂-substituted heteroaryl, OSO₂-heterocyclic, and OSO₂ substituted heterocyclic, wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

The term “aminocarbonyloxy” refers to the group —OC(O)NRR where each R is independently hydrogen, alkyl, substituted alkyl, aryl, heteroaryl, or heterocyclic wherein alkyl, substituted alkyl, aryl, heteroaryl and heterocyclic are as defined herein.

“Thiol” refers to the group —SH.

“Thioxo” or the term “thioketo” refers to the atom (═S).

“Alkylthio” or the term “thioalkoxy” refers to the group —S-alkyl, wherein alkyl is as defined herein. In certain embodiments, sulfur may be oxidized to —S(O)—. The sulfoxide may exist as one or more stereoisomers.

The term “substituted thioalkoxy” refers to the group —S-substituted alkyl.

The term “thioaryloxy” refers to the group aryl-S— wherein the aryl group is as defined herein including optionally substituted aryl groups also defined herein.

The term “thioheteroaryloxy” refers to the group heteroaryl-S— wherein the heteroaryl group is as defined herein including optionally substituted aryl groups as also defined herein.

The term “thioheterocyclooxy” refers to the group heterocyclyl-S— wherein the heterocyclyl group is as defined herein including optionally substituted heterocyclyl groups as also defined herein.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰,

trihalomethyl, —CN, —OCN, —SCN, —NO, —NO₂, ═N₂, —N₃, —SO₂R⁷⁰, —SO₂O
M⁺, —SO₂OR⁷⁰, —OSO₂R⁷⁰, —OSO₂O⁻M⁺, —OSO₂OR⁷⁰, —P(O)(O⁻)₂(M⁺)₂, —P(O)(OR⁷⁰)O⁻
M⁺, —P(O)(OR⁷⁰) 2, —C(O)R⁷⁰, —C(S)R⁷⁰, —C(NR⁷⁰)R⁷⁰, —C(O)O⁻
M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺,
—OC(O)OR⁷⁰, —OC(S)OR⁷⁰, —NR⁷⁰C(O)R⁷⁰, —NR⁷⁰C(S)R⁷⁰, —NR⁷⁰CO₂⁻
M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰
and —NR⁷⁰C(NR⁷⁰)NR⁸⁰R⁸⁰, where R⁶⁰ is selected from the group consisting of optionally substituted alkyl, cycloalkyl, heteroalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 5-, 6- or 7-membered heterocycloalkyl which may optionally include from 1 to 4 of the same or different additional heteroatoms selected from the group consisting of O, N and S, of which N may have —H or C₁-C₃ alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the invention and the other a typical counter ion such as chloride, or two ionized compounds disclosed herein can serve as counter ions for such divalent alkali earth ions, or a doubly ionized compound of the invention can serve as the counter ion for such divalent alkali earth ions). As specific examples, —NR⁸⁰R⁸⁰ is meant to include —NH₂, —NH-alkyl, N-pyrrolidinyl, N-piperazinyl, 4N-methyl-piperazin-1-yl and N-morpholinyl.

In addition to the disclosure herein, substituent groups for hydrogens on unsaturated carbon atoms in “substituted” alkene, alkyne, aryl and heteroaryl groups are, unless otherwise

specified, —R⁶⁰, halo, —O⁻M⁺, —OR⁷⁰, —SR⁷⁰, —S⁻M⁺, —NR⁸⁰R⁸⁰,
trihalomethyl, —CF₃, —CN, —OCN, —SCN, —NO, —NO₂, —N₃, —SO₂R⁷⁰, —SO₃⁻
M⁺, —SO₃R⁷⁰, —OSO₂R⁷⁰, —OSO₃⁻M⁺, —OSO₃R⁷⁰, —PO₃⁻²(M⁺)₂, —P(O)(OR⁷⁰)O⁻
M⁺, —P(O)(OR⁷⁰)₂, —C(O)R⁷⁰, —C(S)R⁷⁰, —C(NR⁷⁰)R⁷⁰, —CO₂⁻
M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻
M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻
M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰
and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the groups disclosed with respect to the individual terms herein, substituent groups for hydrogens on nitrogen atoms in “substituted” heteroalkyl and cycloheteroalkyl groups are, unless otherwise specified, —R⁶⁰, —O⁻M⁺, —OR⁷⁰, —SR⁷⁰, —S⁻M⁺, —NR⁸⁰R⁸⁰,

trihalomethyl, —CF₃, —CN, —NO, —NO₂, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —P(O)(O⁻)₂(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰) R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

“Pharmaceutically effective amount” and “therapeutically effective amount” refer to an amount of a compound sufficient to elicit the desired therapeutic effect (e.g., treatment of a specified disorder or disease or one or more of its symptoms and/or prevention of the occurrence of the disease or disorder). In reference to polyglutamine diseases, a pharmaceutically or therapeutically effective amount includes an amount sufficient to, among other things, prevent or cause a reduction of proteinaceous deposits in the brain of a subject.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

Also of interest as active agents for use in embodiments of the methods are prodrugs. Such prodrugs are in general functional derivatives of the compounds that are readily convertible in vivo into the required compounds. Thus, in the methods of the present disclosure, the term “administering” encompasses administering the compound specifically disclosed or with a compound which may not be specifically disclosed, but which converts to the specified compound in vivo after administration to the subject in need thereof. Conventional procedures for the selection and preparation of suitable prodrug derivatives are described, e.g., in Wermuth, “Designing Prodrugs and Bioprecursors” in Wermuth, ed. The Practice of Medicinal Chemistry, 2d Ed., pp. 561-586 (Academic Press 2003). Prodrugs include esters that hydrolyze in vivo (e.g., in the human body) to produce a compound described herein suitable for the methods and compositions of the present disclosure. Suitable ester groups include, without limitation, those derived from pharmaceutically acceptable, aliphatic carboxylic acids, particularly alkanoic, alkenoic, cycloalkanoic and alkanedioic acids, in which each alkyl or alkenyl moiety has no more than 6 carbon atoms. Illustrative esters include formates, acetates, propionates, butyrates, acrylates, citrates, succinates, and ethylsuccinates.

The term “sample” as used herein relates to a material or mixture of materials, typically, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components). In certain embodiments of the method, the sample includes a cell. In some instances of the method, the cell is in vitro. In some instances of the method, the cell is in vivo.

“Patient” refers to human and non-human subjects, especially mammalian subjects.

The term “treating” or “treatment” as used herein means the treating or treatment of a disease or medical condition in a patient, such as a mammal (particularly a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

Other definitions of terms may appear throughout the specification.

Before the present invention is described in greater detail, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

### DETAILED DESCRIPTION

As summarized above, aspects of the present disclosure include G6PD-modulating agents that can activate and/or stabilize a G6PD enzyme and methods for modulating a glucose-6-phosphate dehydrogenase (G6PD) in a sample using such agents. Also provided are methods for treating a subject for a G6PD deficiency-associated condition, that include administering to a subject an effective amount of a G6PD-modulating agent to selectively activate a mutant G6PD and treat the subject.

The present disclosure describes the characterization of one of the most common G6PD mutant enzymes, Canton G6PD, by X-ray crystallography to identify structurally distorted areas in the enzyme that lead to the decreased enzyme activity. Using this enzyme and high throughput screening methods, a class of G6PD-modulating compounds was identified that can act as activators (e.g., chaperones) to activate the enzyme and/or increase the enzyme's stability in cells (e.g., as described herein). The subject G6PD-modulating compounds demonstrate broad spectrum activity with a variety of common G6PD mutant enzymes, indicating that the subject compounds and methods can find use as agents to activate and/or stabilize G6PD enzymes. Taken together, the subject agents and methods provide a therapeutic strategy to treat G6PD deficiency-associated conditions and diseases.

### G6PD-Modulating Agents

Aspects of the present disclosure include G6PD-modulating agents. A G6PD-modulating agent can be dimeric and include two terminal carbocyclic or heterocyclic groups connected via a linker, e.g., a diamino-containing linker. The subject agent can be homodimeric (e.g., symmetrical) or heterodimeric (e.g., include a non-symmetrical linker and/or two different terminal groups). The terminal carbocyclic or heterocyclic groups can include 1-4 rings (e.g., fused rings) that are independently selected from aryl, heteroaryl, saturated or partially unsaturated carbocycle and saturated or partially unsaturated heterocycle. In some cases, the terminal groups are monocyclic or bicyclic groups (e.g., fused bicyclic or bridged bicyclic). In some instances, a terminal carbocyclic group is selected from aryl, substituted aryl group, cycloalkyl and substituted cycloalkyl. In some instances, a terminal heterocyclic group is selected from heteroaryl, substituted heteroaryl group, saturated heterocycle and substituted saturated heterocycle.

The agent can be a diamino-compound, i.e., a compound that includes two amino groups configured apart from each other at a distance of about 4-15 angstroms to provide for a desirable binding interaction with the target G6PD enzyme binding site. The amino groups can be located at any convenient positions of the molecule, such as within the linker or as part of substituent groups appended to the linker or the terminal groups. In some cases, the two amino groups are each aliphatic amines (e.g., primary, secondary, tertiary or quaternary aliphatic amino groups) which are configured at a desirable distance from each other on opposing sides of a dimeric agent. In certain cases, the two amino groups are contained in substituents that are linked to the terminal carbocyclic or heterocyclic groups. In some cases, the two amino groups are located within the linker such that they form part of the backbone on atoms connecting the terminal carbocyclic or heterocyclic groups. As such, in some cases, the agent includes a diamino-containing linker.

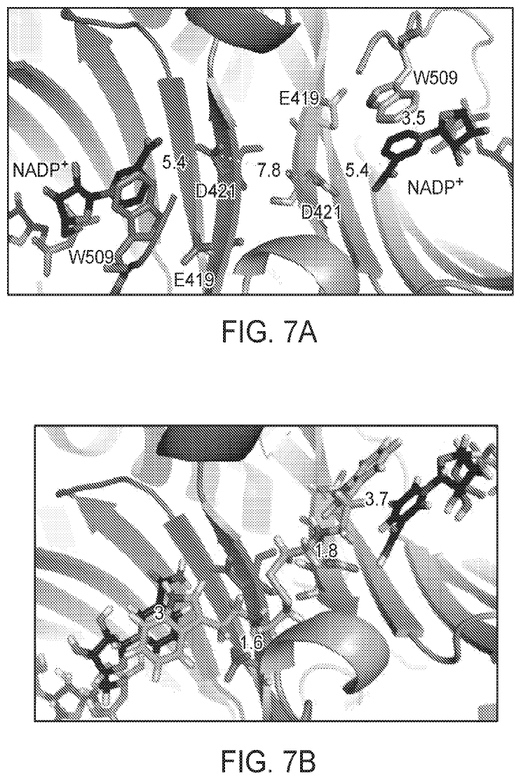

A “diamino containing linker” refers to a divalent linker that connects two moieties and itself includes two amino groups. The two amino groups can be primary, secondary, tertiary or quaternary aliphatic amino groups that are spaced apart from each other by a linkage having a backbone of about 2-20 atoms in length (e.g., 2-18, 2-16, 2-14, 2-12, 4-12 or 4-8 atoms in length). In some cases, the diamino containing linker includes two secondary amino —N(R)— groups in the backbone of the linker, where R is hydrogen, an alkyl or a substituted alkyl. In some cases, the diamino containing linker includes two amino-containing substituents as appendages to the linker. The two amino groups of the subject linker can be spaced apart from each other, e.g., via a linking group or through space, at a distance of about 4-15 angstroms (e.g., 5-10 or 6-8 angstroms, such as about 7 angstroms) and provide for a desirable binding interaction with the target G6PD enzyme binding site (see e.g., FIG. 7B). It is understood that the diamino containing linker can include additional amino group(s) at any convenient positions.

In some embodiments, a G6PD-modulating agent is a diamino compound of formula (I):

Z¹—Y—Z² (I)

wherein:

Z¹ and Z² are independently selected from aryl, substituted aryl, heteroaryl, substituted heteroaryl, carbocycle, substituted carbocycle, heterocycle and substituted heterocycle, wherein optionally Z¹ and Z² are each independently substituted with an amino-containing substituent comprising an amino group; and

Y is a central linking unit, optionally comprising two amino groups separated via a linker; wherein the agent comprises at least two amino groups configured at a distance of about 4-15 angstroms to provide for binding to a G6PD enzyme.

The amino-containing substituent can include a primary, secondary, tertiary or quaternary amino group linked to Z¹ or Z². In some cases, the amino-containing substituent is a linked primary amino group (—NH₂). In some instances of formula (I), Z¹ and Z² are each independently substituted with an amino-containing substituent selected from amino-alkyl, substituted amino-alkyl, amino-alkoxy and substituted amino-alkoxy.

In some embodiments of formula (I), the G6PD-modulating agent is of formula (Ia):

Z¹-T¹-Y-T²-Z² (Ia)

wherein:

Z¹ and Z² are independently selected from aryl, substituted aryl, heteroaryl, substituted heteroaryl, carbocycle, substituted carbocycle, heterocycle and a heterocycle;

T¹ and T² are each independently a covalent bond or a linker; and

Y is a central linking unit comprising two amino groups (i.e., a diamino-containing linker).

In certain embodiments of formula (Ia), the two amino groups of Y are incorporated into the central linking unit backbone and separated by a linker. In certain embodiments of formula (Ia), the two amino groups of Y are part of amino-containing substituents appended to the central linking unit.

In certain embodiments of formula (Ia), Y comprises one of the following structures:

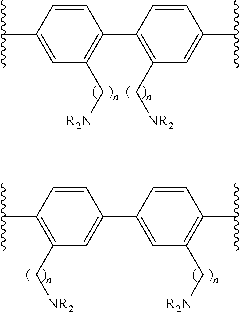

wherein:

each R is independently H, alkyl or substituted alkyl; and

n is 0-6 (e.g., 1, 2 or 3). In some instances of Y, n is 1. In certain cases of Y, n is 2. In certain cases of Y, each R is H.

In some embodiments of formulae (I)-(Ia), Y is a diamino-containing linker and the G6PD-modulating agent is of formula (IIa):

Z¹-T¹-N(R¹)ₓ^{p+}-L¹-N(R²)_{y}^{q+}-T²-Z² (IIa)

wherein:

Z¹ and Z² are independently selected from an aryl, a substituted aryl, a heteroaryl, a substituted heteroaryl, a carbocycle, a substituted carbocycle, a heterocycle and a substituted heterocycle;

T¹ and T² are each independently a covalent bond or a tether;

R¹ and R² are independently H, an alkyl, a substituted alkyl; and

L¹ is a central linker; and

x and y are independently 1 or 2, wherein:
when x is 1, p is 0;
when x is 2, p is 1;
when y is 1, q is 0; and
when y is 2, q is 1.
   In some embodiments of formula (IIa), x and y are each 1, and the agent is of the formula: Z¹-T¹-N(R¹)-L¹-N(R²)-T²-Z². In some instances, R¹ and R² are each H. In some embodiments of formula (IIa), x and y are each 2, and the two amino groups are quaternary amino groups.

In some instances of formula (IIa), L¹ is a linker having a backbone of 2-20 atoms in length (e.g., 2-16, 2-12, 3-12, 4-12 or 4-10, such as 4, 5, 6, 7, 8, 9 or 10 atoms in length), where the linker may be linear, branched or comprise a carbocyclic or heterocyclic group. In certain cases, the linker is a linear alkyl or substituted alkyl where one or two or more carbon atoms of the backbone are optionally substituted with a sulfur or oxygen heteroatom. In certain embodiments of formula (IIa), the agent comprises a diamino-containing linker of one of the following structures:

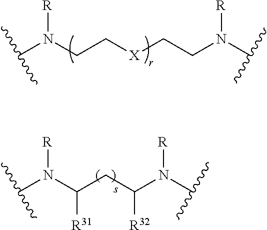

wherein:

each R is independently H, alkyl or substituted alkyl;

r is 0, 1 or 2; and

s is 0-12 (e.g., 1-6 or 2-6, such as 2, 3, 4, 5 or 6).

In some instances of formula (IIa), L¹ is a linker comprising a bivalent carbocyclic or heterocyclic group, optionally further substituted, and linked to the adjacent amino groups directly or via a C1-C6 alkyl or substituted alkyl linker. Bivalent carbocyclic or heterocyclic groups of interest which find use in L¹ include, but are not limited to, cyclobutane, cyclopentane, cyclohexane, naphthalene, quinoline, indole, benzofuran, benzothiophene, benzisooxazole, and substituted versions thereof. In certain embodiments of formula (IIa), the agent comprises a diamino-containing linker of one of the following structures:

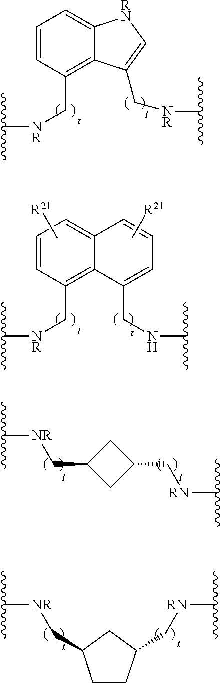

wherein:

each R is independently H, alkyl or substituted alkyl;

each t is 0-6 (e.g., 1, 2 or 3); and

each R²¹ is independently one or more substituents selected from H, alkyl, substituted alkyl, halogen (e.g., chloro, bromo or fluoro), hydroxy, alkoxy, substituted alkoxy, cyano, nitro, formyl (—CHO), sulfonic acid, carboxylic acid, sulfonamide or carobxyamide.

In some embodiments of formula (I), Z¹ and Z² are each independently substituted with an amino-containing substituent and the G6PD-modulating agent is of formula (Ib):

N(R¹)ₓ^{p+}-T¹-Z-L²-Z-T²-N(R²)_{y}^{q+} (Ib)

wherein:

Z¹ and Z² are independently selected from an aryl, a substituted aryl, a heteroaryl, a substituted heteroaryl, a saturated carbocycle, a substituted saturated carbocycle, a heterocycle and a substituted heterocycle;

T¹ and T² are each independently a covalent bond or a linker;

R¹ and R² are independently H, an alkyl, a substituted alkyl; and

L² is a central linker; and

x and y are independently 2 or 3, wherein:
when x is 2, p is 0;
when x is 3, p is 1;
when y is 2, q is 0; and
when y is 3, q is 1.
   In some embodiments of formula (Ib), x and y are each 2, and the agent is of the formula:

N(R¹)₂-T¹-Z¹-L²-Z²-T²-N(R²)₂.

In some embodiments of formula (Ib), x and y are each 3, and the two amino groups are quaternary amino groups.

In some instances of formula (Ib), L² is a linker having a backbone of 2-20 atoms in length (e.g., 2-16, 2-12, 3-12, 4-12 or 4-10, such as 4, 5, 6, 7, 8, 9 or 10 atoms in length), where the linker may be linear, branched or comprise a carbocyclic or heterocyclic group. In certain cases, the linker is a linear alkyl or substituted alkyl where one or two or more carbon atoms of the backbone are optionally substituted with a sulfur or oxygen heteroatom. In some cases of formula (Ib), L² is 2-12 atoms in length. In certain cases, the linker L² is an alkyl or substituted alkyl linker. In certain cases, the linker L² is a linear alkyl or substituted alkyl where one or two or more carbon atoms of the backbone are optionally substituted with a sulfur or oxygen heteroatom. In some instances, L² does not include an amino group.

L² can be a conjugated linker composed of co-monomer groups that for a rigid linking structure between Z¹ and Z² and provides for a desirable separation of the two amino groups. In some instances of formula (Ib), L² has a pi conjugated backbone composed of co-monomer groups such as arylene groups, heteroarylene groups, vinylene, ethynylene, and the like. In some cases, L² is a conjugated linker composed of 1-6 (e.g., 2-6 or 2-4, such as 2, 3 or 4) pi conjugated co-monomer groups selected from 1,4-phenylene, 1,3-phenylene, 2,5-pyridyl, 2,6-pyridyl, fluorene, vinylene, ethynylene, carbazole, a C2-C12 alkyne. In certain cases, L² includes arylene-ethynylene, a heteroarylene-ethynylene, ethynylene and/or 4,4′-biphenyl. In some cases, L² is selected from 4,4′-biphenyl, ethynylene-1,4-phenylene-ethynylene, 1,4-phenylene-ethynylene-1,4-phenylene and substituted versions thereof.

In some embodiments of formula (Ia), the two amino groups of Y are substituents of the central linking unit, and the G6PD-modulating agent is of formula (IIb):

Z¹-T¹-(NHetN)-T²-Z² (IIb)

wherein: —(NHetN)— is a bivalent heterocyclic ring system having 1 to 4 rings (e.g., 5 and/or 6-membered saturated or unsaturated rings, linked in a spiro, fused or conjugated configuration) and comprising two linking terminal N atoms that are linked to T¹ and T², respectively (e.g., a first tertiary amino group connected to T¹ and a second tertiary amino group connected to T²). In some instances of formula (IIb), —(NHetN)— is selected from one of the following structures:

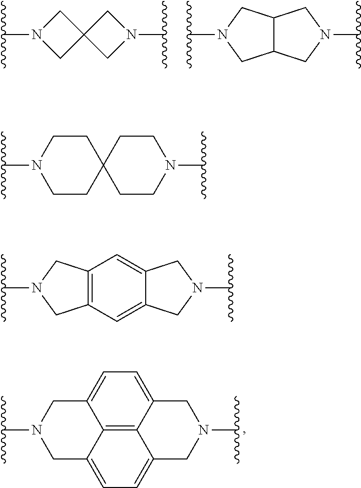

or a substituted version thereof.

In certain embodiments of formula (I)-(IIb), Z¹ and Z² are the same group. The agent can be a symmetric homodimer, e.g., C2 symmetric homodimer. In certain embodiments of formula (I)-(IIb), Z¹ and Z² are different groups. In certain instances of formula (I)-(IIb), Z¹ and Z² are each independently selected from aryl, substituted aryl, heteroaryl and substituted heteroaryl. Z¹ and Z² can be monocyclic, bicyclic or tricyclic groups that are aromatic or partially unsaturated. In certain instances of formula (I)-(IIb), Z¹ and Z² are independently selected from indole, substituted indole, benzofuran, substituted benzofuran, benzothiophene, substituted benzothiophene, phenyl, substituted phenyl, quinoline, substituted quinoline, 1,3-benzodioxole, substituted 1,3-benzodioxole, thiophene, substituted thiophene, 2,3-dihydro-1H-indene, substituted 2,3-dihydro-1H-indene, pyridyl and substituted pyridyl. In certain instances of formula (I)-(IIb), Z¹ and Z² are each independently indole or substituted indole. In some cases, Z¹ and Z² are each independently 3-indolyl or substituted 3-indolyl. In certain instances of formula (I)-(IIb), Z¹ and Z² are each independently benzofuran or substituted benzofuran. In some cases, Z¹ and Z² are each independently substituted benzofuran-3-yl or benzofuran-3-yl. In certain instances of formula (I)-(IIb), Z¹ and Z² are each independently phenyl or substituted phenyl. In certain instances of formula (I)-(IIb), Z¹ and Z² are each independently quinoline or substituted quinoline. In certain instances of formula (I)-(IIb), Z¹ and Z² are each independently a 1,3-benzodioxole or a substituted 1,3-benzodioxole.

In certain instances of formulae (I), (Ia), (IIa) and (IIb), Z¹ and Z² are independently selected from the following: 4-pyridyl, substituted 4-pyridyl (e.g., R²¹ substituted), 3-pyridyl, substituted 3-pyridyl (e.g., R²¹ substituted), 3-pyridyl, substituted 3-pyridyl (e.g., R²¹ substituted), 2-thiophenyl, substituted 2-thiophenyl (e.g., R²¹ substituted),

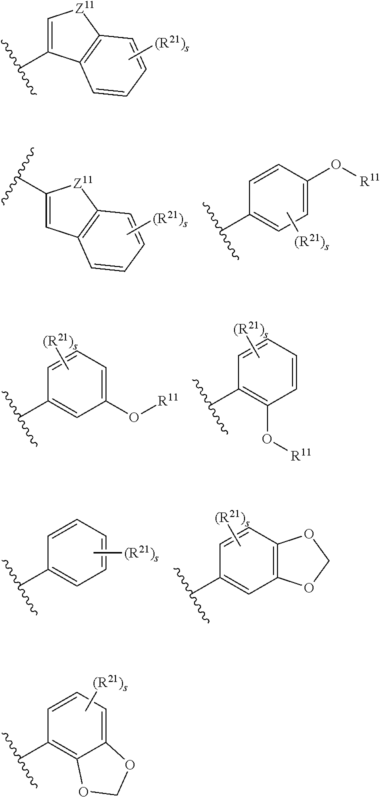

wherein:

Z¹¹ is O, S or NR, wherein R is H, alkyl or substituted alkyl;

s is 0-4 (e.g., 0, 1 or 2);

each R²¹ is independently alkyl, substituted alkyl, halogen (e.g., chloro, bromo or fluoro), hydroxy, alkoxy, substituted alkoxy, cyano, nitro, formyl (—CHO), sulfonic acid, carboxylic acid, sulfonamide or carobxyamide; and

R¹¹ is hydrogen, alkyl or substituted alkyl.

In certain instances of formula (I) and (Ib), Z¹ and Z² are independently selected from the following:

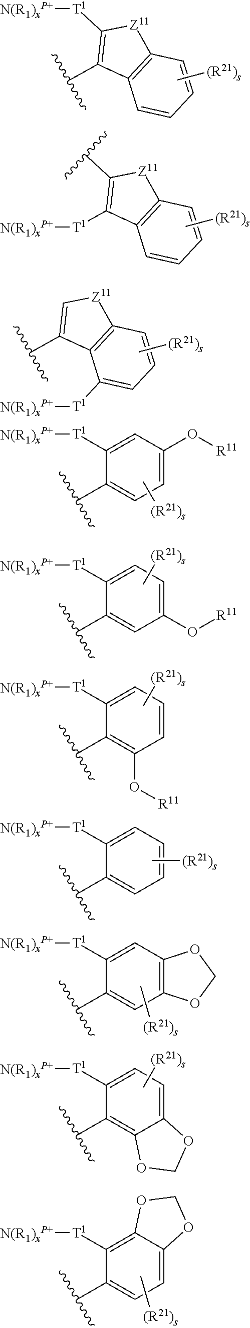

wherein:

Z¹¹ is O, S or NR, wherein R is H, alkyl or substituted alkyl;

s is 0-4 (e.g., 0, 1 or 2);

each R²¹ is independently alkyl, substituted alkyl, halogen (e.g., chloro, bromo or fluoro), hydroxy, alkoxy, substituted alkoxy, cyano, nitro, formyl (—CHO), sulfonic acid, carboxylic acid, sulfonamide or carobxyamide; and

R¹¹ is hydrogen, alkyl or substituted alkyl. In some cases of Z¹ and Z², x is 2 and p is 0. In some cases of Z¹ and Z², each R¹ is H. In some cases of Z¹ and Z², each R¹ is lower alkyl.

In certain instances of formulae (I) and (Ib), Z¹ and Z² are independently selected from:

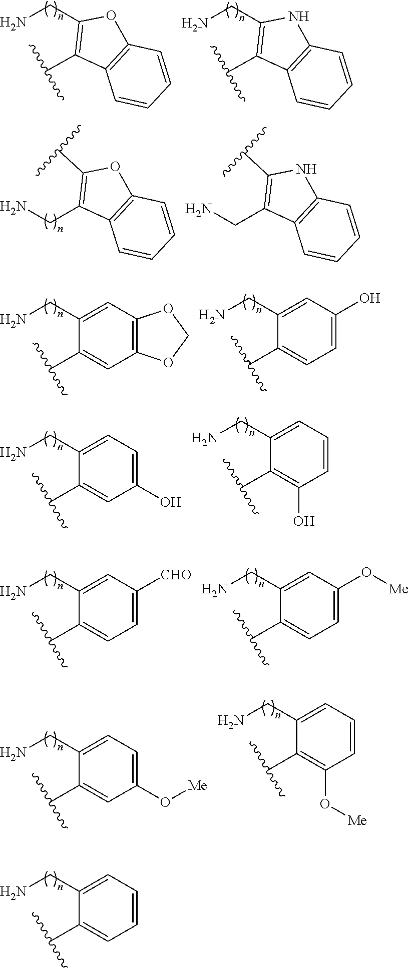

wherein n is 0-6 (e.g., 1, 2 or 3). In some instances of Z¹ and Z², n is 1. In certain cases of Z¹ and Z², n is 2.

In certain instances of formulae (I), (Ia), (Ha) and (IIb), Z¹ and Z² are independently selected from:

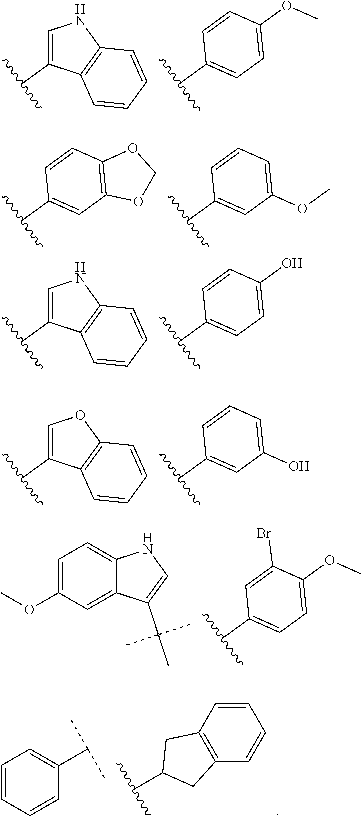

In formula (IIa), the group —N(R¹)ₓ^{p+}-L¹-N(R²)^{q}+— can define the diamino-containing linker. In formula (IIb), the groups N(R¹)ₓ^{p+}-T¹- and -T²-N(R²)_{y}^{q+} can define the amino-containing substituents. In formula (Ia), when Y is substituted with two amino-containing substituents, the substituents can be defined by the formula N(R¹)ₓ^{p+}-T¹- and -T²-N(R²)_{y}^{q+}, respectively (e.g., as defined herein). In certain instances of formula (I)-(Ib), T¹ and T² are themselves each independently a linker of 2-20 atoms in length. In certain instances of formula (I)-(Ib), T¹ and T² are each independently a lower alkyl or a substituted lower alkyl, e.g., (C₁-C₆)alkyl. In certain instances of formula (I)-(Ib), T¹ and T² are each independently a (C₁-C₁₂)alkyl, such as a (C₂-C₁₂)alkyl or (C₂-C₆)alkyl, optionally further substituted. In certain cases of formula (I)-(Ib), T¹ and T² are the same. In certain cases of formula (I)-(Ib), T¹ and T² are each independently —(CH₂)ₘ— where m is 1-6, such as 1, 2, 3, 4, 5 or 6. In certain cases, m is 1, 2 or 3. In some instances, m is 2-6, such as 2 or 3. In some instances, m is 1.

In some embodiments of formulae (Ia) and (IIa), Z¹-T¹- and Z²-T²- are each independently selected from:

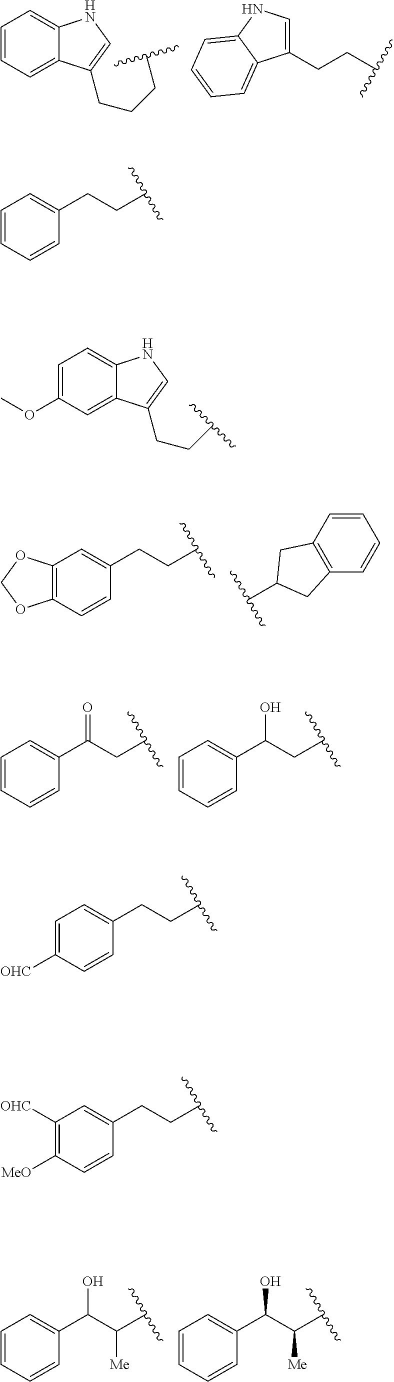

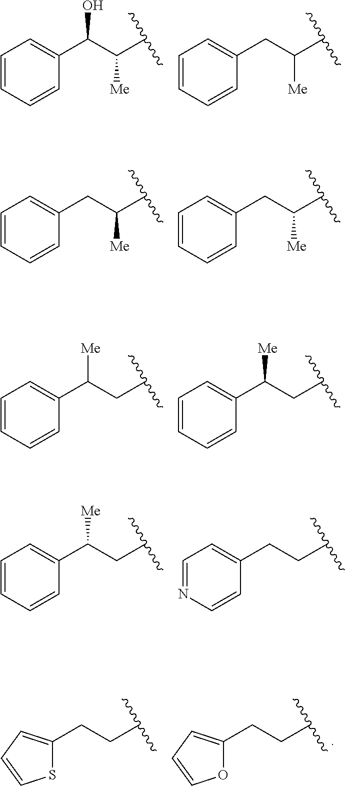

One or both of the amino groups of the diamino-containing linker can, in some cases, be secondary amino groups. As such, in certain embodiments of formula (I), x and y are each 1 and R¹ and R² are each H. One or both of the amino groups of the diamino-containing linker can, in some cases, be tertiary amino groups. As such, in certain embodiments of formula (I), x and y are each 1 and R¹ and R² are each alkyl or substituted alkyl. It is understood that a secondary or tertiary amino group can be further protonated, e.g., under physiological or aqueous conditions. One or both of the amino groups of the diamino-containing linker can, in some cases, be quaternary amino groups. As such, in certain embodiments of formula (I), x and y are each 2 and R¹ and R² are each alkyl or substituted alkyl. In certain embodiments of formula (I), R¹ and R² are each H. In certain cases of formula (I), R¹ and R² are each independently an alkyl or a substituted alkyl. In certain cases of formula (I), R¹ and R² are each independently a lower alkyl or a substituted lower alkyl. In certain instances of formula (I), R¹ and R² are each methyl.

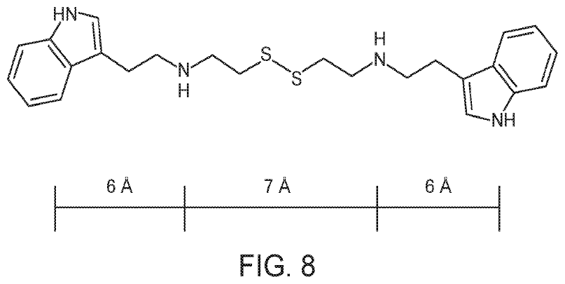

In some instances of formula (I), L is a central linker having a backbone that is 2-20 atoms in length. In certain cases, L has a backbone of 2-12 atoms in length, such as 2-6, 3-20, 3-12, 3-6, 4-20, 4-12 or 4-6 atoms in length. In certain instances, L is a linker that provides for an intramolecular spacing between the nitrogen atoms of the two amino groups of 4-15 angstroms in length, such as 5-10 or 6-8 angstroms, e.g., about 7 angstroms. FIG. 8 depicts an exemplary compound where approximate intramolecular distance between the two amino groups of the linker is illustrated.

In some embodiments of formula (IIa), L¹ is of the formula (III):

-L¹¹-Z³-L¹²- (III)

wherein:

L¹¹ and L¹² are independently alkyl, substituted alkyl or a polyethylene glycol (PEG) moiety; and

Z³ is selected from a covalent bond, a heteroatom, a cycloalkyl, an aryl, a heteroaryl, a bicyclic carbocycle, a cubane, an alkenyl, an allenyl, an alkynyl and a cleavable group.

In some instances of formula (III), L¹¹ and L¹² are independently a (C₁-C₁₂)alkyl or a substituted (C₁-C₁₂)alkyl. In some instances of formula (III), L¹¹ and L¹² are independently a (C₂-C₁₂)alkyl or a substituted (C₂-C₁₂)alkyl, such as a (C₂-C₆)alkyl or a substituted (C₂-C₆)alkyl. In certain cases of formula (III), L¹¹ and L¹² are each independently —(CH₂)— where n is 2-6, such as 2, 3, 4, 5 or 6. In some instances of formula (III), L¹¹ and L¹² are independently a polyethylene glycol (PEG) moiety, e.g., —CH₂CH₂O— or —OCH₂CH₂—.

In some instances of formula (III), Z³ is a covalent bond and L¹¹ and L¹² together define a single linker, e.g., an alkyl linker or a polyethylene glycol (PEG) linker. In certain instances of formula (IIa), L¹ is —(CH₂)— wherein n is 2-12. In some instances of formula (III), Z³ is a heteroatom, e.g., —O— or —S—. In some instances of formula (IIa), L¹ is —(CH₂)ₙ—X—(CH₂)ₘ— wherein X is O or S and n and m are each independently 2-6, such as 2, 3, 4, 5 or 6. In some instances of formula (III), Z³ is a cycloalkyl. In some cases of formula (III), Z³ is a cyclohexyl, e.g., a 1,4-cyclohexyl. In some cases of formula (III), Z³ is a cyclopentyl, e.g., a 1,3-cyclopentyl. In some cases of formula (III), Z³ is a cyclobutyl, e.g., a 1,3-cyclobutyl. In some cases of formula (III), Z³ is an aryl or a substituted aryl. In certain cases of formula (III), Z³ is a phenyl or substituted phenyl, such as a 1,4-phenyl, a 1,3-phenyl or a 1,2-phenyl. In some cases of formula (III), Z³ is a heteroaryl or substituted heteroaryl. In some cases of formula (III), Z³ is a pyridyl, e.g., a 2,6-pyridyl. In some cases of formula (III), Z³ is a bicyclic carbocycle, e.g., a naphthalenyl, an indole or a bicycle[1,1,1]pentane. In some cases of formula (III), Z³ is a cubane. In some cases of formula (III), Z³ is an alkenyl, an allenyl or an alkynyl, optionally substituted.

In some embodiments of formula (III), Z³ includes a cleavable group. Any convenient cleavable groups can be adapted for use in the subject linkers. In some cases, Z³ is a cleavable group that can be cleaved via application of a stimulus, e.g., contact with a chemical or redox reagent, a light photon or an enzyme), to change the nature of the compound and alter its binding properties with the target G6DP. Cleavable groups of interest include, but are not limited to, a chemically-cleavable moiety, an enzyme-cleavable moiety, a protease-cleavable moiety, an oxidatively-cleavable moiety, and a photocleavable moiety. In certain instances, Z³ is an ester, e.g., —C(O)O—. In certain instances, Z³ is a disulfide, e.g., —SS—.

In certain instances of formula (IIa), L¹ is selected from:

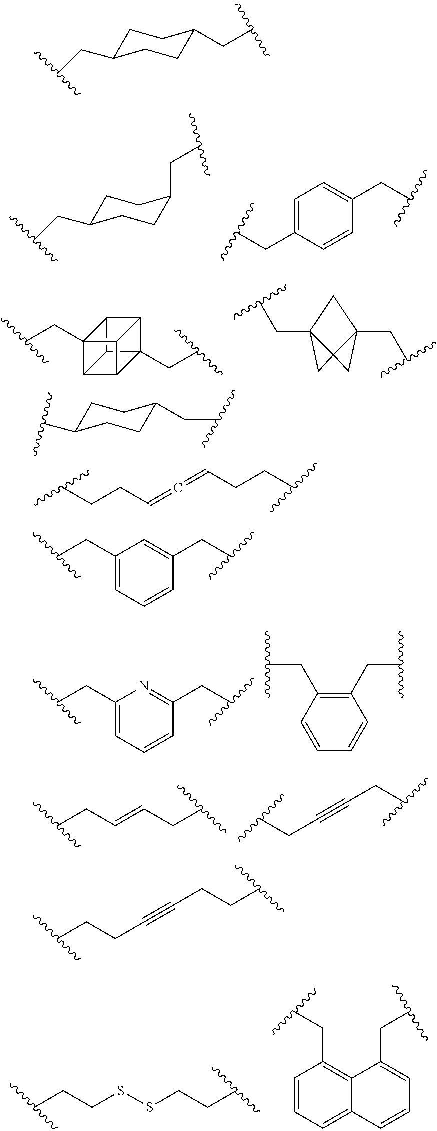

In certain embodiments of formula (IIa), L¹ is selected from one of the following structures:

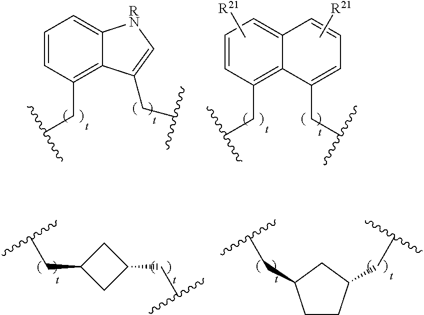

wherein each t is 0-6 (e.g., 1, 2 or 3); and each R is independently one or more substituents selected from H, alkyl, substituted alkyl, halogen (e.g., chloro, bromo or fluoro), hydroxy, alkoxy, substituted alkoxy, cyano, nitro, formyl (—CHO), sulfonic acid, carboxylic acid, sulfonamide or carobxyamide.

In certain instances of formula (IIa), Z¹ and Z² are independently selected from one of following combinations:

| Z¹ | Z² |
|---|---|
| 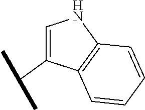 | 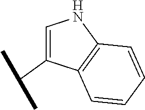 |
| 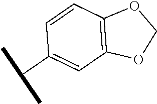 | 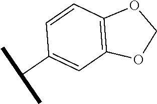 |
| 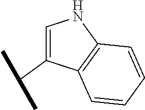 | 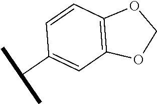 |
| 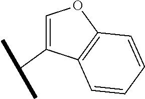 | 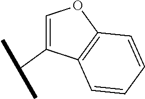 |
| 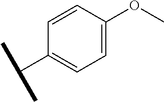 | 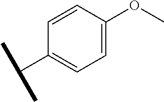 |
| 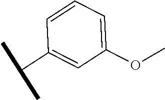 | 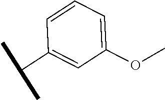 |
| 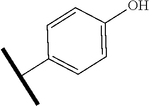 | 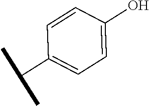 |
| 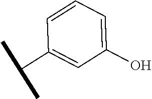 | 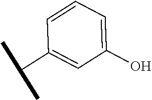 |
| 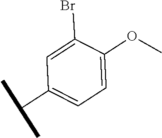 | 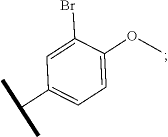 |

and L¹ is selected from:

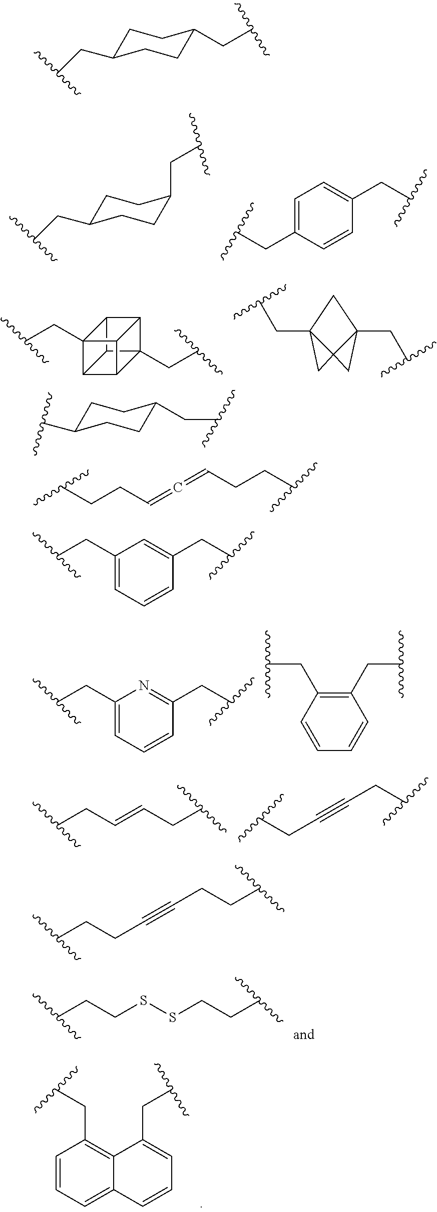

In certain instances of any one of the embodiments above, T¹ and T² are each independently —(CH₂)ₘ— where m is 1-6, such as 1, 2, 3, 4, 5 or 6; and x and y are each 1 and R¹ and R² are each H.

In some embodiments, the G6PD-modulating agent is of the formula:

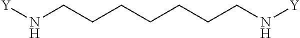

wherein each Y is selected from:

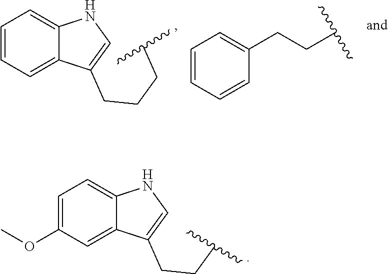

In some embodiments, the G6PD-modulating agent is of the formula:

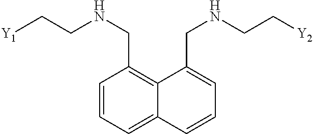

wherein Y₁ and Y₂ are selected from one of the following combinations:

| Y₁ | Y₂ |
|---|---|
| 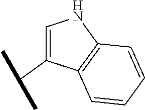 | 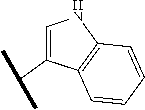 |
| 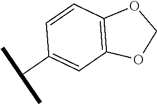 | 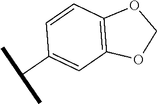 |
| 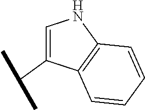 | 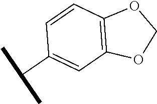 |
| 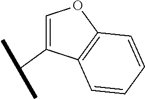 | 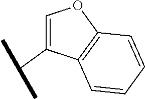 |
| 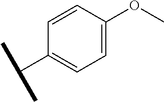 | 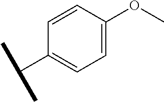 |
| 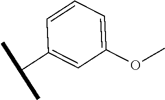 | 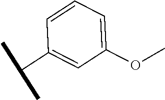 |
| 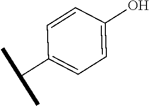 | 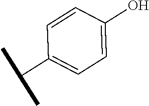 |
| 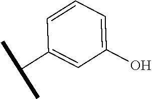 | 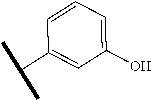 |
| 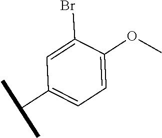 | 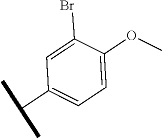 |

In some embodiments, the G6PD-modulating agent is of the formula:

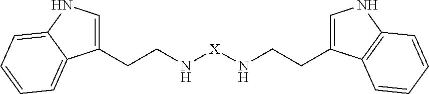

wherein X is selected from:

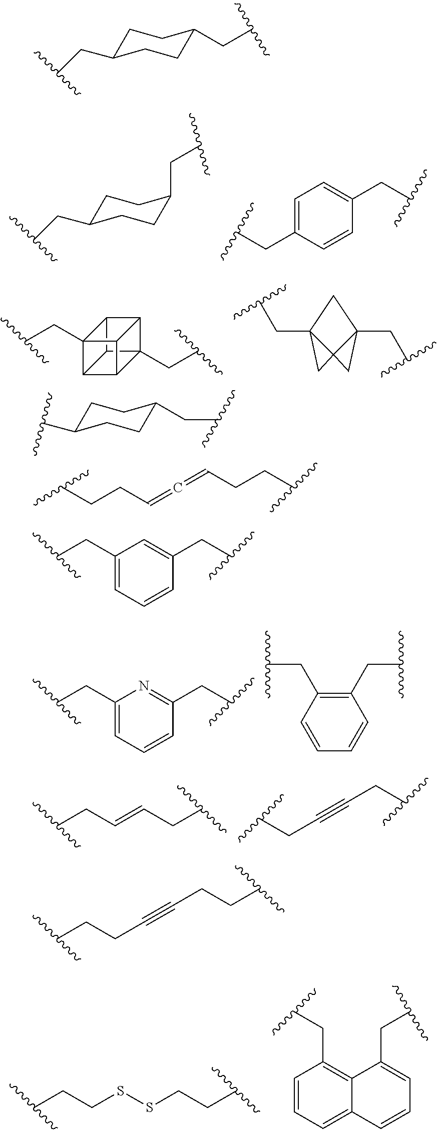

—(CH₂)₂—, —(CH₂)₃—, —(CH₂)₄—, —(CH₂)₅—, —(CH₂)₆—, —(CH₂)₇—, —(CH₂)—, —(CH₂)₉—, —(CH₂)₁₀— and —(CH₂)₂—.

In some instances, the agent is a compound of one of Tables 1-4, such as one of compounds 1-47.

TABLE 1
Compounds of formula (Ib)

| Compound | Structure |
|---|---|
| 1 | 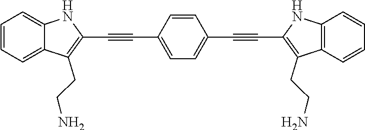 [SMILES: NCCc1c(C#Cc2ccc(C#Cc3[nH]c4ccccc4c3CCN)cc2)[nH]c2ccccc12] |
| 2 | 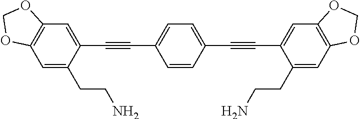 [SMILES: NCCc1cc2c(cc1C#Cc1ccc(C#Cc3cc4c(cc3CCN)OCO4)cc1)OCO2] |
| 3 | 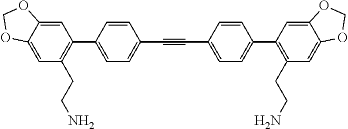 [SMILES: NCCc1cc2c(cc1-c1ccc(C#Cc3ccc(-c4cc5c(cc4CCN)OCO5)cc3)cc1)OCO2] |
| 4 | 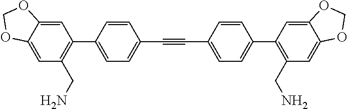 [SMILES: NCc1cc2c(cc1-c1ccc(C#Cc3ccc(-c4cc5c(cc4CN)OCO5)cc3)cc1)OCO2] |
| 5 | 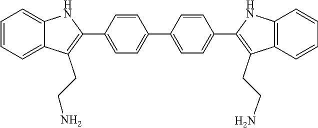 [SMILES: NCCc1c(-c2ccc(-c3ccc(-c4[nH]c5ccccc5c4CCN)cc3)cc2)[nH]c2ccccc12] |
| 6 | 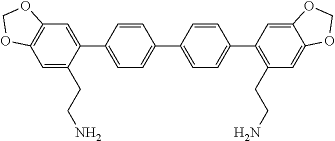 [SMILES: NCCc1cc2c(cc1-c1ccc(-c3ccc(-c4cc5c(cc4CCN)OCO5)cc3)cc1)OCO2] |
| 7 | 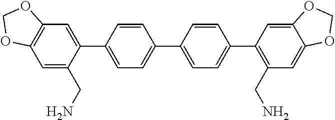 [SMILES: NCc1cc2c(cc1-c1ccc(-c3ccc(-c4cc5c(cc4CN)OCO5)cc3)cc1)OCO2] |
| 8 | 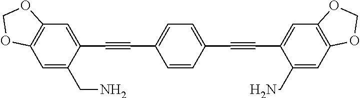 [SMILES: NCc1cc2c(cc1C#Cc1ccc(C#Cc3cc4c(cc3CN)OCO4)cc1)OCO2] |
| 9 | 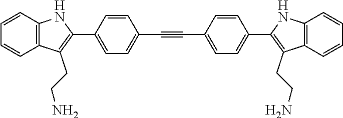 [SMILES: NCCc1c(-c2ccc(C#Cc3ccc(-c4[nH]c5ccccc5c4CCN)cc3)cc2)[nH]c2ccccc12] |
| 10 | 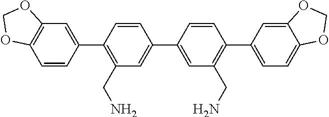 [SMILES: NCc1cc(-c2ccc(-c3ccc4c(c3)OCO4)c(CN)c2)ccc1-c1ccc2c(c1)OCO2] |
| 11 | 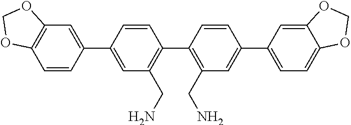 [SMILES: NCc1cc(-c2ccc3c(c2)OCO3)ccc1-c1ccc(-c2ccc3c(c2)OCO3)cc1CN] |

TABLE 2
Compounds of formula (IIa)

| Compound | Structure |
|---|---|
| 12 | 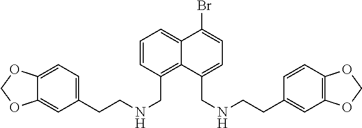 [SMILES: Brc1ccc(CNCCc2ccc3c(c2)OCO3)c2c(CNCCc3ccc4c(c3)OCO4)cccc12] |
| 13 | 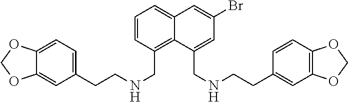 [SMILES: Brc1cc(CNCCc2ccc3c(c2)OCO3)c2c(CNCCc3ccc4c(c3)OCO4)cccc2c1] |
| 14 | 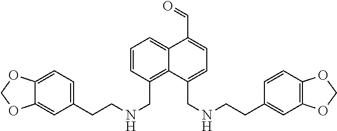 [SMILES: O=Cc1ccc(CNCCc2ccc3c(c2)OCO3)c2c(CNCCc3ccc4c(c3)OCO4)cccc12] |
| 15 | 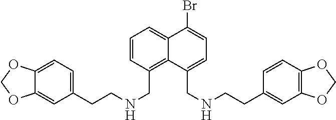 [SMILES: Brc1ccc(CNCCc2ccc3c(c2)OCO3)c2c(CNCCc3ccc4c(c3)OCO4)cccc12] |
| 16 | 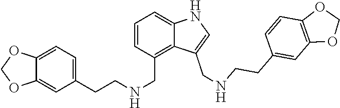 [SMILES: c1cc(CNCCc2ccc3c(c2)OCO3)c2c(CNCCc3ccc4c(c3)OCO4)c[nH]c2c1] |
| 17 | 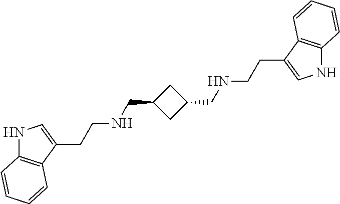 [SMILES: c1ccc2c(CCNC[C@H]3C[C@H](CNCCc4c[nH]c5ccccc45)C3)c[nH]c2c1] |
| 18 | 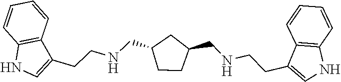 [SMILES: c1ccc2c(CCNC[C@H]3CC[C@H](CNCCc4c[nH]c5ccccc45)C3)c[nH]c2c1] |

TABLE 3
Compounds of formula (IIa)

| Compound | Structure |
|---|---|
| 19 | 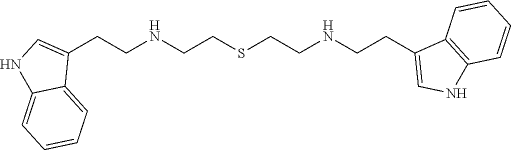 [SMILES: c1ccc2c(CCNCCSCCNCCc3c[nH]c4ccccc34)c[nH]c2c1] |
| 20 | 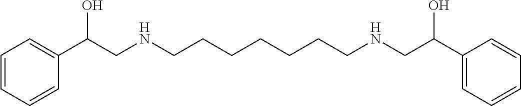 [SMILES: OC(CNCCCCCCCNCC(O)c1ccccc1)c1ccccc1] |
| 21 | 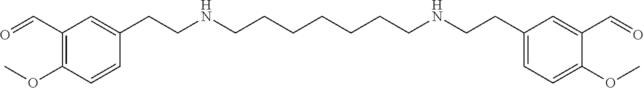 [SMILES: COc1ccc(CCNCCCCCCCNCCc2ccc(OC)c(C=O)c2)cc1C=O] |
| 22 | 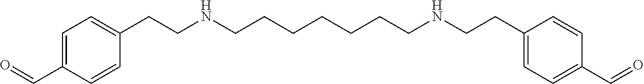 [SMILES: O=Cc1ccc(CCNCCCCCCCNCCc2ccc(C=O)cc2)cc1] |
| 23 | 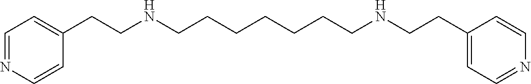 [SMILES: c1cc(CCNCCCCCCCNCCc2ccncc2)ccn1] |
| 24 | 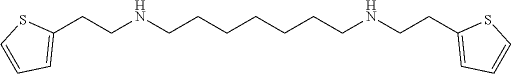 [SMILES: c1csc(CCNCCCCCCCNCCc2cccs2)c1] |
| 25 | 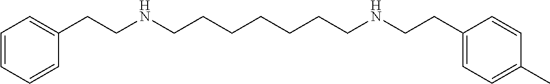 [SMILES: Cc1ccc(CCNCCCCCCCNCCc2ccccc2)cc1] |
| 26 | 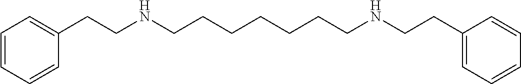 [SMILES: c1ccc(CCNCCCCCCCNCCc2ccccc2)cc1] |
| 27 | 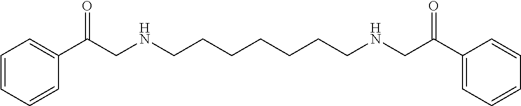 [SMILES: O=C(CNCCCCCCCNCC(=O)c1ccccc1)c1ccccc1] |
| 28 | 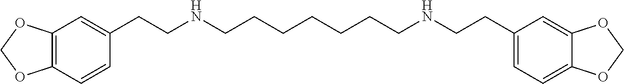 [SMILES: c1cc2c(cc1CCNCCCCCCCNCCc1ccc3c(c1)OCO3)OCO2] |
| 29 | 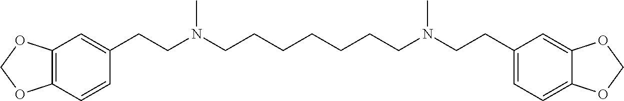 [SMILES: CN(CCCCCCCN(C)CCc1ccc2c(c1)OCO2)CCc1ccc2c(c1)OCO2] |
| 30 | 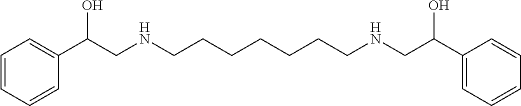 [SMILES: OC(CNCCCCCCCNCC(O)c1ccccc1)c1ccccc1] |
| 31 | 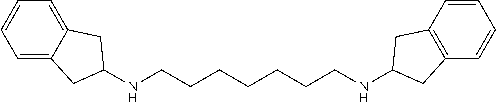 [SMILES: c1ccc2c(c1)CC(NCCCCCCCNC1Cc3ccccc3C1)C2] |
| 32 | 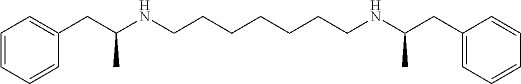 [SMILES: C[C@H](Cc1ccccc1)NCCCCCCCN[C@@H](C)Cc1ccccc1] |
| 33 | 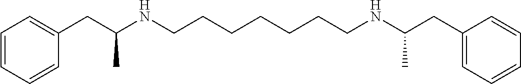 [SMILES: C[C@@H](Cc1ccccc1)NCCCCCCCN[C@@H](C)Cc1ccccc1] |
| 34 | 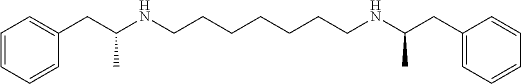 [SMILES: C[C@H](Cc1ccccc1)NCCCCCCCN[C@H](C)Cc1ccccc1] |
| 35 | 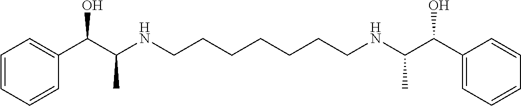 [SMILES: C[C@H](NCCCCCCCN[C@@H](C)[C@H](O)c1ccccc1)[C@H](O)c1ccccc1] |
| 36 | 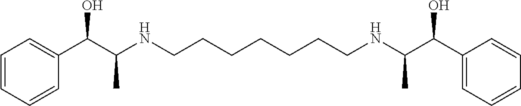 [SMILES: C[C@H](NCCCCCCCN[C@H](C)[C@@H](O)c1ccccc1)[C@H](O)c1ccccc1] |
| 37 | 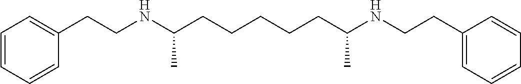 [SMILES: C[C@H](CCCCC[C@H](C)NCCc1ccccc1)NCCc1ccccc1] |
| 38 | 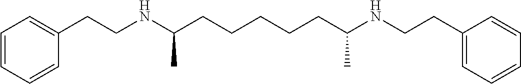 [SMILES: C[C@H](CCCCC[C@@H](C)NCCc1ccccc1)NCCc1ccccc1] |
| 39 | 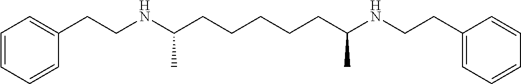 [SMILES: C[C@@H](CCCCC[C@H](C)NCCc1ccccc1)NCCc1ccccc1] |
| 40 | 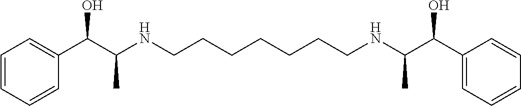 [SMILES: C[C@H](NCCCCCCCN[C@H](C)[C@@H](O)c1ccccc1)[C@H](O)c1ccccc1] |
| 41 | 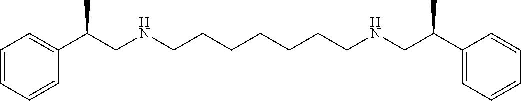 [SMILES: C[C@H](CNCCCCCCCNC[C@H](C)c1ccccc1)c1ccccc1] |
| 42 | 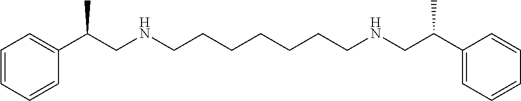 [SMILES: C[C@@H](CNCCCCCCCNC[C@H](C)c1ccccc1)c1ccccc1] |
| 43 | 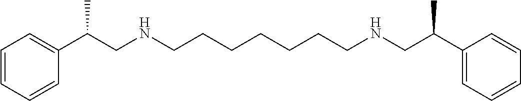 [SMILES: C[C@H](CNCCCCCCCNC[C@@H](C)c1ccccc1)c1ccccc1] |

TABLE 4
Compounds of formula (IIb)

| Compound | Structure |
|---|---|
| 44 | 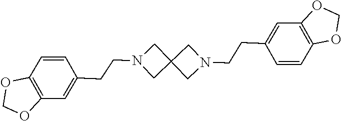 [SMILES: c1cc2c(cc1CCN1CC3(C1)CN(CCc1ccc4c(c1)OCO4)C3)OCO2] |
| 45 | 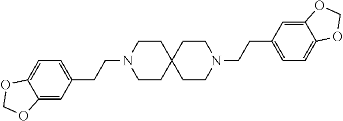 [SMILES: c1cc2c(cc1CCN1CCC3(CC1)CCN(CCc1ccc4c(c1)OCO4)CC3)OCO2] |
| 46 | 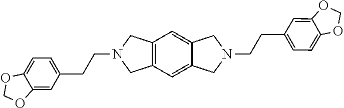 [SMILES: c1cc2c(cc1CCN1Cc3cc4c(cc3C1)CN(CCc1ccc3c(c1)OCO3)C4)OCO2] |
| 47 | 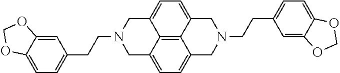 [SMILES: c1cc2c(cc1CCN1Cc3ccc4c5c(ccc(c35)C1)CN(CCc1ccc3c(c1)OCO3)C4)OCO2] |

In some embodiments, the G6PD-modulating agent is NOT 2-[2-(1H-indol-3-yl)ethylamino]ethanethiol (AG1).

Aspects of the present disclosure include a G6PD-modulating agents (e.g., as described herein), salts thereof (e.g., pharmaceutically acceptable salts), and/or solvate, hydrate and/or prodrug forms thereof. In addition, it is understood that, in any compound described herein having one or more chiral centers, if an absolute stereochemistry is not expressly indicated, then each center may independently be of R-configuration or S-configuration or a mixture thereof. It will be appreciated that all permutations of salts, solvates, hydrates, prodrugs and stereoisomers are meant to be encompassed by the present disclosure.

In some embodiments, the subject G6PD-modulating agents, or a prodrug form thereof, are provided in the form of pharmaceutically acceptable salts. Compounds containing an amine or nitrogen containing heteroaryl group may be basic in nature and accordingly may react with any number of inorganic and organic acids to form pharmaceutically acceptable acid addition salts. Acids commonly employed to form such salts include inorganic acids such as hydrochloric, hydrobromic, hydriodic, sulfuric and phosphoric acid, as well as organic acids such as para-toluenesulfonic, methanesulfonic, oxalic, para-bromophenylsulfonic, carbonic, succinic, citric, benzoic and acetic acid, and related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephathalate, sulfonate, xylenesulfonate, phenylacetate, phenylpropionate, phenylbutyrate, citrate, lactate, 3-hydroxybutyrate, glycollate, maleate, tartrate, methanesulfonate, propanesulfonates, naphthalene-1-sulfonate, naphthalene-2-sulfonate, mandelate, hippurate, gluconate, lactobionate, and the like salts. In certain specific embodiments, pharmaceutically acceptable acid addition salts include those formed with mineral acids such as hydrochloric acid and hydrobromic acid, and those formed with organic acids such as fumaric acid and maleic acid.

In some embodiments, the subject G6PD-modulating agents are provided in a prodrug form. “Prodrug” refers to a derivative of an active agent that requires a transformation within the body to release the active agent. In certain embodiments, the transformation is an enzymatic transformation. Prodrugs are frequently, although not necessarily, pharmacologically inactive until converted to the active agent. “Promoiety” refers to a form of protecting group that, when used to mask a functional group within an active agent, converts the active agent into a prodrug. In some cases, the promoiety will be attached to the drug via bond(s) that are cleaved by enzymatic or non-enzymatic means in vivo. Any convenient prodrug forms of the subject compounds can be prepared, e.g., according to the strategies and methods described by Rautio et al. (“Prodrugs: design and clinical applications”, Nature Reviews Drug Discovery 7, 255-270 (February 2008)).

In some embodiments, the subject G6PD-modulating agents, prodrugs, stereoisomers or salts thereof are provided in the form of a solvate (e.g., a hydrate). The term “solvate” as used herein refers to a complex or aggregate formed by one or more molecules of a solute, e.g. a prodrug or a pharmaceutically-acceptable salt thereof, and one or more molecules of a solvent. Such solvates are typically crystalline solids having a substantially fixed molar ratio of solute and solvent. Representative solvents include by way of example, water, methanol, ethanol, isopropanol, acetic acid, and the like. When the solvent is water, the solvate formed is a hydrate.

### Pharmaceutical Preparations

Also provided are pharmaceutical preparations. Pharmaceutical preparations are compositions that include a G6PD-modulating agent (e.g., as described herein) (for example one or more of the subject compounds, either alone or in the presence of one or more additional active agents) present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the present disclosure is formulated for administration to a mammal. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents may be used.

When administered to a mammal, the compounds and compositions of the present disclosure and pharmaceutically acceptable vehicles, excipients, or diluents may be sterile. In some instances, an aqueous medium is employed as a vehicle when the subject compound is administered intravenously, such as water, saline solutions, and aqueous dextrose and glycerol solutions.

Pharmaceutical compositions can take the form of capsules, tablets, pills, pellets, lozenges, powders, granules, syrups, elixirs, solutions, suspensions, emulsions, suppositories, or sustained-release formulations thereof, or any other form suitable for administration to a mammal. In some instances, the pharmaceutical compositions are formulated for administration in accordance with routine procedures as a pharmaceutical composition adapted for oral or intravenous administration to humans. Examples of suitable pharmaceutical vehicles and methods for formulation thereof are described in Remington: The Science and Practice of Pharmacy, Alfonso R. Gennaro ed., Mack Publishing Co. Easton, Pa., 19th ed., 1995, Chapters 86, 87, 88, 91, and 92, incorporated herein by reference. The choice of excipient will be determined in part by the particular compound, as well as by the particular method used to administer the composition. Accordingly, there is a wide variety of suitable formulations of the subject pharmaceutical compositions.

Administration of the subject G6PD-modulating agents may be systemic or local. In certain embodiments administration to a mammal will result in systemic release of a compound of the present disclosure (for example, into the bloodstream). Methods of administration may include enteral routes, such as oral, buccal, sublingual, and rectal; topical administration, such as transdermal and intradermal; and parenteral administration. Suitable parenteral routes include injection via a hypodermic needle or catheter, for example, intravenous, intramuscular, subcutaneous, intradermal, intraperitoneal, intraarterial, intraventricular, intrathecal, and intracameral injection and non-injection routes, such as intravaginal rectal, or nasal administration. In certain embodiments, the compounds and compositions of the present disclosure are administered subcutaneously. In certain embodiments, the compounds and compositions of the present disclosure are administered orally. In certain embodiments, it may be desirable to administer one or more compounds of the present disclosure locally to the area in need of treatment. This may be achieved, for example, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers.

The compounds can be formulated into preparations for injection by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives.

A subject G6PD-modulating agent may also be formulated for oral administration. For an oral pharmaceutical formulation, suitable excipients include pharmaceutical grades of carriers such as mannitol, lactose, glucose, sucrose, starch, cellulose, gelatin, magnesium stearate, sodium saccharine, and/or magnesium carbonate. For use in oral liquid formulations, the composition may be prepared as a solution, suspension, emulsion, or syrup, being supplied either in solid or liquid form suitable for hydration in an aqueous carrier, such as, for example, aqueous saline, aqueous dextrose, glycerol, or ethanol, preferably water or normal saline. If desired, the composition may also contain minor amounts of non-toxic auxiliary substances such as wetting agents, emulsifying agents, or buffers. In some embodiments, formulations suitable for oral administration can include (a) liquid solutions, such as an effective amount of the compound dissolved in diluents, such as water, or saline; (b) capsules, sachets or tablets, each containing a predetermined amount of the active ingredient, as solids or granules; (c) suspensions in an appropriate liquid; and (d) suitable emulsions. Tablet forms can include one or more of lactose, mannitol, corn starch, potato starch, microcrystalline cellulose, acacia, gelatin, colloidal silicon dioxide, croscarmellose sodium, talc, magnesium stearate, stearic acid, and other excipients, colorants, diluents, buffering agents, moistening agents, preservatives, flavoring agents, and pharmacologically compatible excipients. Lozenge forms can include the active ingredient in a flavor, usually sucrose and acacia or tragacanth, as well as pastilles including the active ingredient in an inert base, such as gelatin and glycerin, or sucrose and acacia, emulsions, gels, and the like containing, in addition to the active ingredient, such excipients as are described herein.

The subject formulations can be made into aerosol formulations to be administered via inhalation. These aerosol formulations can be placed into pressurized acceptable propellants, such as dichlorodifluoromethane, propane, nitrogen, and the like. They may also be formulated as pharmaceuticals for non-pressured preparations such as for use in a nebulizer or an atomizer.

In some embodiments, formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Formulations suitable for topical administration may be presented as creams, gels, pastes, or foams, containing, in addition to the active ingredient, such carriers as are appropriate. In some embodiments the topical formulation contains one or more components selected from a structuring agent, a thickener or gelling agent, and an emollient or lubricant. Frequently employed structuring agents include long chain alcohols, such as stearyl alcohol, and glyceryl ethers or esters and oligo(ethylene oxide) ethers or esters thereof. Thickeners and gelling agents include, for example, polymers of acrylic or methacrylic acid and esters thereof, polyacrylamides, and naturally occurring thickeners such as agar, carrageenan, gelatin, and guar gum. Examples of emollients include triglyceride esters, fatty acid esters and amides, waxes such as beeswax, spermaceti, or carnauba wax, phospholipids such as lecithin, and sterols and fatty acid esters thereof. The topical formulations may further include other components, e.g., astringents, fragrances, pigments, skin penetration enhancing agents, sunscreens (e.g., sunblocking agents), etc.

Unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier.

The term “unit dosage form,” as used herein, refers to physically discrete units suitable as unitary dosages for human and animal subjects, each unit containing a predetermined quantity of compounds of the present disclosure calculated in an amount sufficient to produce the desired effect in association with a pharmaceutically acceptable diluent, carrier or vehicle. The specifications for the novel unit dosage forms of the present disclosure depend on the particular compound employed and the effect to be achieved, and the pharmacodynamics associated with each compound in the host. In pharmaceutical dosage forms, the compounds may be administered in the form of a free base, their pharmaceutically acceptable salts, or they may also be used alone or in appropriate association, as well as in combination, with other pharmaceutically active compounds.

Dose levels can vary as a function of the specific compound, the nature of the delivery vehicle, and the like. Desired dosages for a given compound are readily determinable by a variety of means. The dose administered to an animal, particularly a human, in the context of the present disclosure should be sufficient to effect a prophylactic or therapeutic response in the animal over a reasonable time frame, e.g., as described in greater detail herein. Dosage will depend on a variety of factors including the strength of the particular compound employed, the condition of the animal, and the body weight of the animal, as well as the severity of the illness and the stage of the disease. The size of the dose will also be determined by the existence, nature, and extent of any adverse side-effects that might accompany the administration of a particular compound.

### Methods of Modulating G6PD

Aspects of the present disclosure include methods of modulating a glucose-6-phosphate dehydrogenase (G6PD) in a sample by contacting the sample with a G6PD-modulating agent (e.g., as described herein). The G6PD-modulating agent can act to stabilize the G6PD and/or modulate (e.g., activate) the G6PD enzyme. In some cases, the subject agent binds the G6PD at a NADP⁺ binding site of the enzyme thereby structurally stabilizing the enzyme. FIGS. 7A and 7B illustrates an expanded view of the X-ray crystal structure of the Canton variant (R459L) of G6PD focused at the G6PD dimer interface and a proposed binding site of the subject agents, where the structural NADP⁺ is shown as black sticks (FIG. 7A) and an exemplary compound is shown in cyan (FIG. 7B). The majority of the G6PD variants that cause severe or mild deficiency are primarily located/mutated in those functional regions of the enzyme, disturbing the enzyme's activity and stability.

In certain embodiments, the deleterious impact that is reduced by subject methods may be loss of function of a G6PD. In certain of these embodiments, the wild-type or normal activity of the G6PD is at least partially, if not completely, impaired because the variant G6PD is structurally destabilized. In these instances, the loss of function is at least partially, if not completely, reversed by binding of the subject agent. The desired function of the target G6PD may be enhanced by a statistically significant amount as compared to a suitable control, e.g., a sample or cell not contacted with the agent of interest, where the magnitude of the enhancement in desired activity may be 10% or more, such as 20% or more, 30% or more, 40% or more, 50% or more, 60% or more, 70% or more, 80% or more, 90% or more, 100% or more, or even more.

By “structurally stabilizing” is meant that binding of the subject compound leads to a folded state of the G6PD protein having enhanced stability (e.g., enhanced thermal stability and/or enhanced stability against enzymatic degradation), which can change one or more properties of the enzyme. In some cases, structurally stabilizing the G6PD restores the activity of a G6PD mutant, or increases a catalytic activity of the enzyme (e.g., activates the G6PD).

By “enhanced thermal stability” is meant the temperature at which the G6PD enzyme retains half of its catalytic activity (T_{1/2}) is increased by a statistically significant amount, and in some cases by 2 degrees Celsius or more, such as 3 degrees or more, 4 degrees or more, 5 degrees or more, 6 degrees or more, 8 degrees or more, 10 degrees or more, 15 degrees or more, 20 degrees or more, or even more, relative to a control G6PD that is not contacted with the agent.

By “enhanced stability against enzymatic degradation” is meant the half-life of the G6PD to chymotrypsin degradation is increased by 10% or more, such as 20% or more, 30% or more, 40% or more, 50% or more, 60% or more, 70% or more, 80% or more, 90% or more, 100% or more, or even more, relative to a control G6PD that is not contacted with the agent.

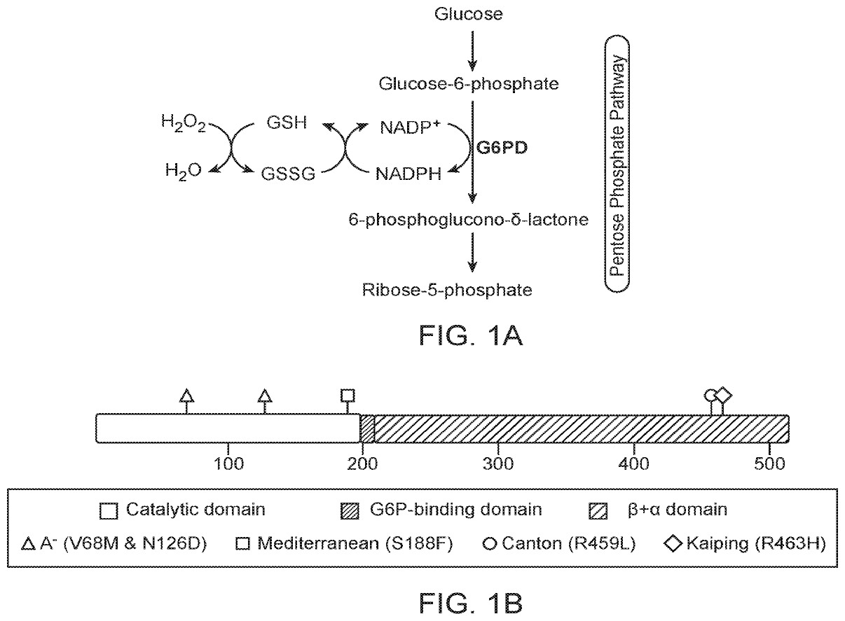

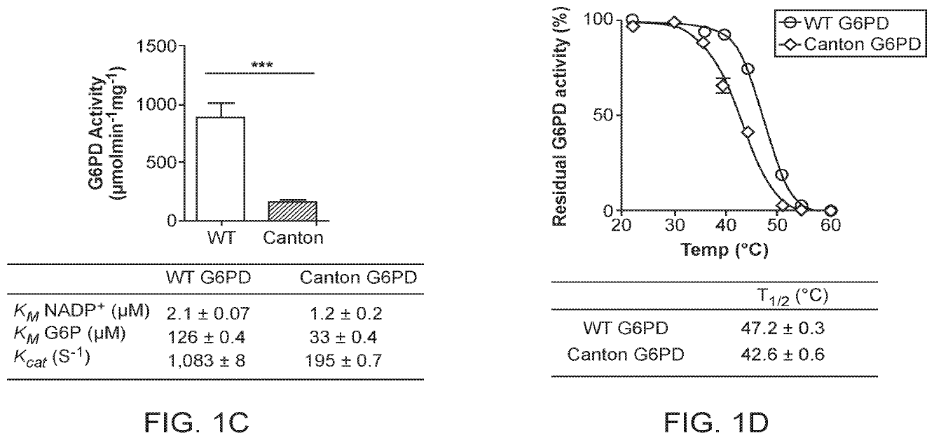

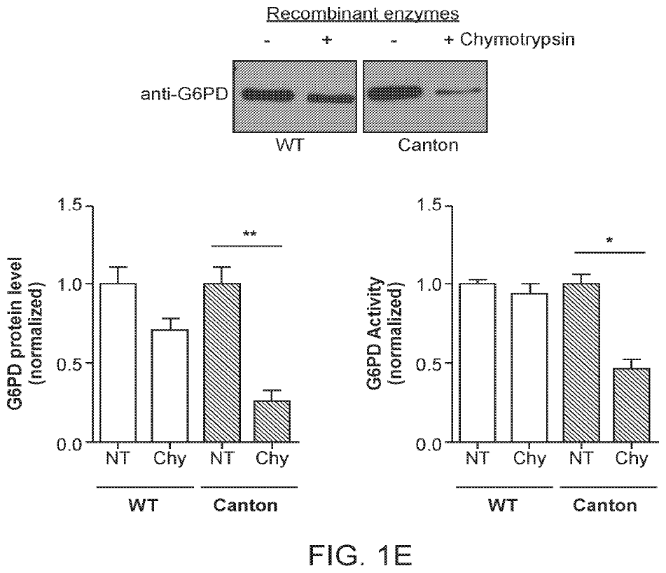

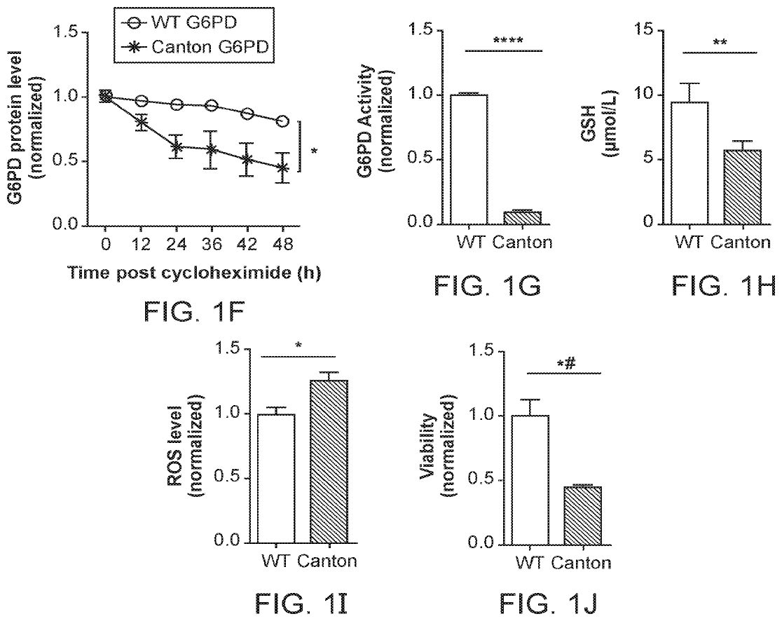

By “activates the G6PD” is meant that the level of enzymatic activity of the G6PD (see, e.g., FIG. 1A), is enhanced by a statistically significant amount, and in some cases by 10% or more, such as 20% or more, 30% or more, 40% or more, 50% or more, 60% or more, 70% or more, 80% or more, 90% or more, 100% or more, or even more, relative to a control G6PD that is not contacted with the agent. It is understood that the level of enzymatic activity of the G6PD can be measured directly (e.g., via consumption of substrate or production of enzymatic product) or indirectly (e.g., via levels of glutathione (GSH), NADPH, and/or measures of oxidative stress on a cell).

In certain embodiments, the subject method further includes a step of assessing the level of activity of the G6PD in the sample. Any convenient assays may be used to determine an enhanced stability or activation of G6PD in a sample using the subject agents relative to a control, e.g., a sample not contacted with the compound of interest, where the magnitude of the change may be 10% or more, such as 20% or more, 30% or more, 50% or more, 100% or more, such as by 2-fold or more, by 5-fold or more, by 10-fold or more, by 20-fold or more, by 50-fold or more, by 100-fold or more, or even more. As such, where the agent activates the G6PD, the activity level can be 110% or more such as 120% or more, 130% or more, 140% or more, 150% or more, 150% or more, 150% or more, 150% or more, 150% or more, 150% or more, or even more, by comparison to a control, e.g., a sample not contacted with the agent. Any convenient direct to indirect markers of G6PD activity, and any convenient assays may be utilized to determine the level of function or activity of a G6PD of interest in a sample.

In some cases, the G6PD is a G6PD variant or mutant that disturbs the enzyme's activity and stability. Any convenient G6PD variant can be targeted according to the subject methods. In such cases, the subject agent can maintain or restore the stability and/or activity of the G6PD to that of a corresponding wild-type, G6PD. Accordingly, in these embodiments the subject methods may maintain or restore a physiologically desirable activity (e.g., wild-type G6PD activity) of the target G6PD despite the presence of the harmful mutation. In certain cases, the activity of a normal allele of the target G6PD is maintained in the sample, e.g., has an activity that is within 20% (such as within 10%, within 5%, within 2% or within 1%) of the corresponding activity of a control sample not contacted with the subject agent.

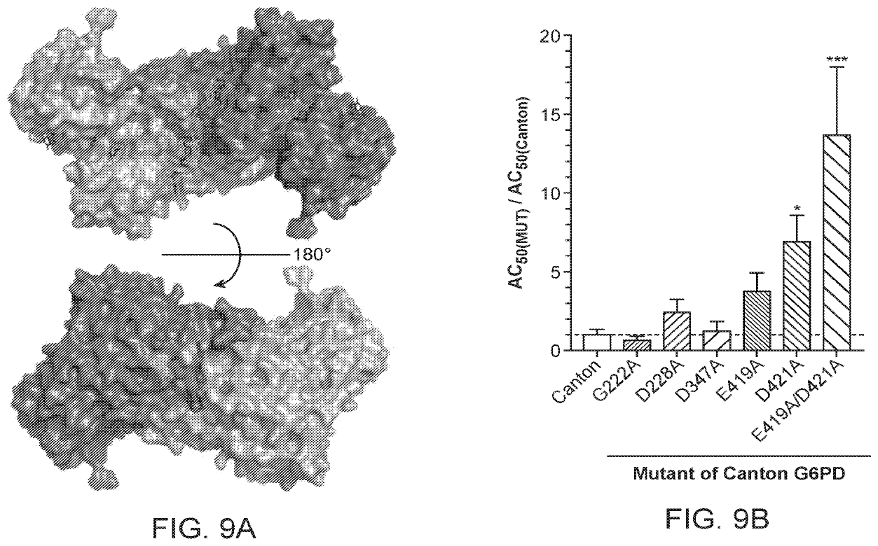

In certain embodiments, the G6PD is a mutant G6PD including a mutation at one or more of the activity-influencing positions, e.g., positions as identified in the X-ray structure analysis described herein and illustrated in FIGS. 9A and 9B. In some cases, the mutation is located at R459, V68, N126, S188, R463 and/or P172. In certain instances, the mutant G6PD is a Canton G6PD mutant. A Canton G6PD mutant refers to a G6PD that includes a R459L mutation and optionally one or more additional mutations. In certain cases, the Canton G6PD mutant is the variant including only R459L.

In certain embodiments of the method, the deleterious impact of the variant G6PD is increased vulnerability to oxidative damage, e.g., to a cell. As such, in some instances the subject methods result in a reduction in oxidative damage or susceptibility to oxidative damage, that is attributable to the target G6PD variant, where the magnitude of the reduction may vary, and in some instances is 2-fold or more, such as by 5-fold or more, by 10-fold or more, by 20-fold or more, by 50-fold or more, by 100-fold or more, or even more. e.g., as compared to a suitable control, e.g., a cell not contacted with the agent of interest. As described in greater detail below, oxidative damage may be reduced in a number of different ways that may depend on the particular target G6PD. Oxidative damage can be assessed directly or indirectly (e.g., level of NADPH and/or GSH in a cell). In some cases, the sample is a cellular sample and the subject methods result in an increase in cell viability, such as an increase of 5% or more, such as 10% or more, such as 15% or more, 20% or more, 25% or more, 30% or more, or even more, relative to a control. In some instances, the oxidative damage is reduced by maintaining a desired level of NADPH and/or GSH in the sample, e.g., a cell. Level of NADPH and/or GSH may be assayed using any convenient protocol, e.g., as described in the experimental section below.

In certain embodiments, the subject agents increase the viability of the cell, as compared to a suitable control and as determined by a cell viability assay, e.g., as determined by contacting the cell with a compound of the present disclosure to a cell and determining the number of viable cells in culture using a homogeneous method, such as the CellTiter-Glo® Luminescent Cell Viability Assay.

The term “sample” relates to a material or mixture of materials, in some cases, although not necessarily, in fluid, i.e., aqueous, form, containing one or more components of interest. Samples may be derived from a variety of sources such as from food stuffs, environmental materials, a biological sample or solid, such as tissue or fluid isolated from an individual, including but not limited to, for example, plasma, serum, spinal fluid, semen, lymph fluid, the external sections of the skin, respiratory, intestinal, and genitourinary tracts, tears, saliva, milk, blood cells, tumors, organs, and also samples of in vitro cell culture constituents (including but not limited to conditioned medium resulting from the growth of cells in cell culture medium, putatively virally infected cells, recombinant cells, and cell components).

In some cases, the sample is a cellular sample. Any convenient cells may be targeted for use in the subject methods. In some instances, the types of cells in which the compound exhibit activity are ones that include a target G6PD, e.g., a variant G6PD. In some embodiments of the method, the cell is an animal cell or a yeast cell. In certain instances, the cell is a mammalian cell.

In practicing methods according to certain embodiments, an effective amount of the compound, e.g., G6PD-modulating agent, is provided to the target cell or cells. In some instances, the effective amount of the compound is provided in the cell by contacting the cell with the compound. Contact of the cell with the modulatory agent may occur using any convenient protocol. The protocol may provide for in vitro or in vivo contact of the modulatory agent with the target cell, depending on the location of the target cell. In some instances, the cell is in vitro. In certain instances, the cell is in vivo. Contact may or may not include entry of the compound into the cell. For example, where the target cell is an isolated cell, the modulatory agent may be introduced directly into the cell under cell culture conditions permissive of viability of the target cell. The choice of method is generally dependent on the type of cell being contacted and the nature of the compound, and the circumstances under which the transformation is taking place (e.g., in vitro, ex vivo, or in vivo).

Alternatively, where the target cell or cells are part of a multicellular organism, the modulatory agent may be administered to the organism or subject in a manner such that the compound is able to contact the target cell(s), e.g., via an in vivo or ex vivo protocol. By “in vivo”, is meant the agent is administered to a living body of an animal. By “ex vivo” it is meant that cells or organs are modified outside of the body. Such cells or organs are in some cases returned to a living body.

In certain embodiments, the method is an in vivo method that includes: administering to a subject in need thereof an effective amount of a subject G6PD-modulating agent (e.g., as described herein) that selectively to activate a mutant G6PD and treat the subject for the G6PD deficiency-associated condition. The term “treating” or “treatment” as used herein means the treating or treatment of a disease or medical condition in a patient, such as a mammal (such as a human) that includes: (a) preventing the disease or medical condition from occurring, such as, prophylactic treatment of a subject; (b) ameliorating the disease or medical condition, such as, eliminating or causing regression of the disease or medical condition in a patient; (c) suppressing the disease or medical condition, for example by, slowing or arresting the development of the disease or medical condition in a patient; or (d) alleviating a symptom of the disease or medical condition in a patient.

As used herein, the terms “host”, “subject”, “individual” and “patient” are used interchangeably and refer to any mammal in need of such treatment according to the disclosed methods. Such mammals include, e.g., humans, ovines, bovines, equines, porcines, canines, felines, non-human primate, mice, and rats. In certain embodiments, the subject is a non-human mammal. In some embodiments, the subject is a farm animal. In other embodiments, the subject is a pet. In some embodiments, the subject is mammalian. In certain instances, the subject is human. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

The amount of compound administered can be determined using any convenient methods to be an amount sufficient to produce the desired effect in association with a pharmaceutically acceptable diluent, carrier or vehicle. The specifications for the unit dosage forms of the present disclosure will depend on the particular compound employed and the effect to be achieved, and the pharmacodynamics associated with each compound in the host.

In some embodiments, an effective amount of a subject compound is an amount that ranges from about 50 ng/ml to about 50 μg/ml (e.g., from about 50 ng/ml to about 40 μg/ml, from about 30 ng/ml to about 20 μg/ml, from about 50 ng/ml to about 10 μg/ml, from about 50 ng/ml to about 1 μg/ml, from about 50 ng/ml to about 800 ng/ml, from about 50 ng/ml to about 700 ng/ml, from about 50 ng/ml to about 600 ng/ml, from about 50 ng/ml to about 500 ng/ml, from about 50 ng/ml to about 400 ng/ml, from about 60 ng/ml to about 400 ng/ml, from about 70 ng/ml to about 300 ng/ml, from about 60 ng/ml to about 100 ng/ml, from about 65 ng/ml to about 85 ng/ml, from about 70 ng/ml to about 90 ng/ml, from about 200 ng/ml to about 900 ng/ml, from about 200 ng/ml to about 800 ng/ml, from about 200 ng/ml to about 700 ng/ml, from about 200 ng/ml to about 600 ng/ml, from about 200 ng/ml to about 500 ng/ml, from about 200 ng/ml to about 400 ng/ml, or from about 200 ng/ml to about 300 ng/ml).

In some embodiments, an effective amount of a subject compound is an amount that ranges from about 10 pg to about 100 mg, e.g., from about 10 pg to about 50 pg, from about 50 pg to about 150 pg, from about 150 pg to about 250 pg, from about 250 pg to about 500 pg, from about 500 pg to about 750 pg, from about 750 pg to about 1 ng, from about 1 ng to about 10 ng, from about 10 ng to about 50 ng, from about 50 ng to about 150 ng, from about 150 ng to about 250 ng, from about 250 ng to about 500 ng, from about 500 ng to about 750 ng, from about 750 ng to about 1 μg, from about 1 μg to about 10 μg, from about 10 μg to about 50 μg, from about 50 μg to about 150 μg, from about 150 μg to about 250 μg, from about 250 μg to about 500 μg, from about 500 μg to about 750 μg, from about 750 μg to about 1 mg, from about 1 mg to about 50 mg, from about 1 mg to about 100 mg, or from about 50 mg to about 100 mg. The amount can be a single dose amount or can be a total daily amount. The total daily amount can range from 10 pg to 100 mg, or can range from 100 mg to about 500 mg, or can range from 500 mg to about 1000 mg.

In some embodiments, a single dose of the subject compound is administered. In other embodiments, multiple doses of the subject compound are administered. Where multiple doses are administered over a period of time, the G6PD-modulating compound is administered twice daily (qid), daily (qd), every other day (qod), every third day, three times per week (tiw), or twice per week (biw) over a period of time. For example, a compound is administered qid, qd, qod, tiw, or biw over a period of from one day to about 2 years or more. For example, a compound is administered at any of the aforementioned frequencies for one week, two weeks, one month, two months, six months, one year, or two years, or more, depending on various factors.

Any of a variety of methods can be used to determine whether a treatment method is effective. For example, a biological sample obtained from an individual who has been treated with a subject method can be assayed for G6PD enzyme activity and/or level of GSH and/or NADPH. Assessment of the effectiveness of the methods of treatment on the subject can include assessment of the subject before, during and/or after treatment, using any convenient methods. Aspects of the subject methods further include a step of assessing the therapeutic response of the subject to the treatment.

In some embodiments, the method includes assessing the condition of the subject, including diagnosing or assessing one or more symptoms of the subject which are associated with the disease or condition of interest being treated (e.g., as described herein). In some embodiments, the method includes obtaining a biological sample from the subject and assaying the sample, e.g., for the G6PD enzyme activity and/or level of GSH and/or NADPH or for the presence of cells that are associated with the disease or condition of interest (e.g., as described herein). The sample can be a cellular sample. The assessment step(s) of the subject method can be performed at one or more times before, during and/or after administration of the subject compounds, using any convenient methods. In certain cases, the assessment step includes identification of cells including a mutant G6PD. In certain instances, assessing the subject includes diagnosing whether the subject has a G6PD-associated disease or condition of interest.

In some instances, the method delays occurrence of a symptom associated with the disease. In certain instances, the method reduces the magnitude of a symptom associated with the disease. Disease conditions of interest include those associated with G6PD deficiency, such as oxidative stress-related conditions. In some cases, a condition associated with G6PD deficiency is manifest as acute haemolysis, which can be characterized by fatigue, back pain, anaemia, and/or jaundice. In addition, several clinical disorders, such as diabetes, myocardial infarction or intense physical exercise can precipitate haemolysis in G6PD-deficient individuals. The term “modify the progression” is employed to encompass both reduction in rate of progression (e.g., as manifested in the delay of the occurrence of one or more symptoms of the disease condition), as well as reversal of progression, including cure, of a disease condition (e.g., as manifested in the reduction of magnitude of one or more symptoms of the disease condition). Specific disease conditions in which the methods and compositions of the invention find use include, but are not limited to, those listed in the Introduction section above, and oxidative stress-related conditions related to Class I, II or III G6PD deficiency, acute haemolysis, hemolytic anemia, bilirubin-induced neurological injury and bilirubin encephalopathy (kernicterus), chronic non-spherocytic hemolytic anemia, intermittent hemolytic episode, a neurological condition, edema, kidney injury and cataract. In certain cases, the condition is selected from bilirubin-induced neurological injury and bilirubin encephalopathy (kernicterus).

A variety of different surrogate markers may be employed to monitor the disease condition and the effect of therapy thereon. Practice of embodiments of the methods can result in improvement in the parameters being measured in the particular test that is employed, where the improvement in some instances is 5% or greater, such as 10% or greater, and in some instances may be 100%, or even greater. In some instances, samples taken from the blood, tissues and body fluids of patients are analyzed for surrogate markers. These markers may vary, where examples of such markers include analytes found in serum or physical measurements, such as pH or blood volume. The concentration, levels, or quantitative measurements of such markers in body fluids and tissues are often found to correspond with the emergence of disease symptoms. Additionally, surrogate markers for disease may be imaging markers, e.g., markers obtained by neuroimaging and magnetic resonance imaging (MRI). Imagining is employed to provide information about volume, levels of atrophy, and activity in white and grey matter across regions of the brain.

In the subject methods, the compound (e.g., as described herein) may be administered to the targeted cells using any convenient administration protocol capable of resulting in the desired activity. Thus, the subject compounds can be incorporated into a variety of formulations, e.g., pharmaceutically acceptable vehicles, for therapeutic administration.

The above methods find use in a variety of different applications. Certain applications are now reviewed in the following Utility section.

### Utility

The subject methods and compound compositions find use in a variety of applications in which stabilizing and/or activating a target G6PD enzyme is desired. As such, aspects of the invention include activating a mutant G6PD using a subject agent, as described herein, in any subject in need thereof, e.g., a subject that has been diagnosed with a G6PD deficiency-associated condition that can be treated by effecting one or more of the above outcomes in the subject. Of interest is use of the subject methods and compositions to modify the progression of disease conditions associated with a G6PD deficiency-associated condition. The phrase “modify the progression” is employed to encompass both reduction in rate of progression (e.g., as manifested in the delay of the occurrence of one or more symptoms of the disease condition), as well as reversal of progression, including cure, of a disease condition (e.g., as manifested in the reduction of magnitude of one or more symptoms of the disease condition). Specific disease conditions in which the methods and compositions of the invention find use include, but are not limited to oxidative stress-related conditions related to Class I, II or III G6PD deficiency, acute haemolysis, hemolytic anemia, bilirubin-induced neurological injury and bilirubin encephalopathy (kernicterus), chronic non-spherocytic hemolytic anemia, intermittent hemolytic episode, a neurological condition, edema, kidney injury and cataract.

Patients having Class I G6PD variants can have severe enzyme deficiency (e.g., <10 percent of normal) associated with chronic hemolytic anemia. Patients having Class II variants also have severe enzyme deficiency (<10 percent of normal), but there is usually only intermittent hemolysis, typically on exposure to oxidant stress such as fava bean exposure or ingestion of certain drugs. G6PD Mediterranean is an example. Patients having Class III variants have moderate enzyme deficiency (e.g., 10 to 60 percent of normal) with intermittent hemolysis, typically associated with significant oxidant stress. G6PD A⁻ is an example.

In some instances, practice of subject methods results in treatment of a subject for a disease condition. By treatment is meant at least an amelioration of one or more symptoms associated with the disease condition afflicting the subject, where amelioration is used in a broad sense to refer to at least a reduction in the magnitude of a parameter, e.g., symptom, associated with the pathological condition being treated, such as loss of cognitive function, etc. As such, treatment also includes situations where the pathological condition, or at least symptoms associated therewith, are completely inhibited, e.g., prevented from happening, or stopped, e.g., terminated, such that the subject no longer suffers from the pathological condition, or at least the symptoms that characterize the pathological condition. Treatment may also manifest in the form of a modulation of a surrogate marker of the disease condition, e.g., as described above.

A variety of hosts are treatable according to the subject methods. Generally such hosts are “mammals” or “mammalian,” where these terms are used broadly to describe organisms which are within the class mammalia, including the orders carnivore (e.g., dogs and cats), rodentia (e.g., mice, guinea pigs and rats), and primates (e.g., humans, chimpanzees and monkeys). In some embodiments, the host is human.

The subject agents, preparations, kits and methods find use in the preservation of cellular samples, e.g., erythrocyte cells, by decreasing the degree of hemolysis of the cells over time and improving preservation during erythrocyte storage. In some cases, the agents provide for storage stability of the cellular sample. In some cases, the preservative preparation finds use in conjunction with blood storage and blood transfusion methods and applications.

### Combination Therapies

The subject compounds can be administered to a subject alone or in combination with an additional, i.e., second, active agent. As such, in some cases, the subject method further comprises administering to the subject at least one additional therapy or compound. Any convenient agents may be utilized, including compounds useful for treating G6PD-deficiency associated conditions or oxidative stress related conditions in general. In some cases, the subject compounds are administered to negate the effect of a drug the subject is taking which causes oxidative stress. Such drugs are known to precipitate hemolysis in G6PD-deficient individuals. In some cases, administration of the subject agent can provide for continued treatment with a drug that causes oxidative stress.

The terms “agent,” “compound,” and “drug” are used interchangeably herein. For example, selective G6PD-modulating agent compounds can be administered alone or in conjunction with one or more other drugs, such as drugs employed in the treatment of oxidative-stress related diseases. In some embodiments, the method further includes coadministering concomitantly or in sequence a second agent. In some embodiments, the method further includes performing a blood transfusion (e.g., exchange transfusion) on the subject.

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

“Concomitant administration” of a known therapeutic drug with a pharmaceutical composition of the present disclosure means administration of the compound and second agent at such time that both the known drug and the composition of the present invention will have a therapeutic effect. Such concomitant administration may involve concurrent (i.e. at the same time), prior, or subsequent administration of the drug with respect to the administration of a subject compound. Routes of administration of the two agents may vary, where representative routes of administration are described in greater detail below. A person of ordinary skill in the art would have no difficulty determining the appropriate timing, sequence and dosages of administration for particular drugs and compounds of the present disclosure.

In some embodiments, the compounds (e.g., a subject compound and the at least one additional compound) are administered to the subject within twenty-four hours of each other, such as within 12 hours of each other, within 6 hours of each other, within 3 hours of each other, or within 1 hour of each other. In certain embodiments, the compounds are administered within 1 hour of each other. In certain embodiments, the compounds are administered substantially simultaneously. By administered substantially simultaneously is meant that the compounds are administered to the subject within about 10 minutes or less of each other, such as 5 minutes or less, or 1 minute or less of each other.

Also provided are pharmaceutical preparations of the subject compounds and the second active agent. In pharmaceutical dosage forms, the compounds may be administered in the form of their pharmaceutically acceptable salts, or they may also be used alone or in appropriate association, as well as in combination, with other pharmaceutically active compounds.

Dosage levels of the order of from about 0.01 mg to about 140 mg/kg of body weight per day are useful in representative embodiments, or alternatively about 0.5 mg to about 7 g per patient per day. Those of skill will readily appreciate that dose levels can vary as a function of the specific compound, the severity of the symptoms and the susceptibility of the subject to side effects. Dosages for a given compound are readily determinable by those of skill in the art by a variety of means.

The amount of active ingredient that may be combined with the carrier materials to produce a single dosage form will vary depending upon the host treated and the particular mode of administration. For example, a formulation intended for the oral administration of humans may contain from 0.5 mg to 5 g of active agent compounded with an appropriate and convenient amount of carrier material which may vary from about 5 to about 95 percent of the total composition. Dosage unit forms will generally contain between from about 1 mg to about 500 mg of an active ingredient, such as 25 mg, 50 mg, 100 mg, 200 mg, 300 mg, 400 mg, 500 mg, 600 mg, 800 mg, or 1000 mg.

It will be understood, however, that the specific dose level for any particular patient will depend upon a variety of factors including the age, body weight, general health, sex, diet, time of administration, route of administration, rate of excretion, drug combination and the severity of the particular disease undergoing therapy.

As such, unit dosage forms for oral or rectal administration such as syrups, elixirs, and suspensions may be provided wherein each dosage unit, for example, teaspoonful, tablespoonful, tablet or suppository, contains a predetermined amount of the composition containing one or more inhibitors. Similarly, unit dosage forms for injection or intravenous administration may include the inhibitor(s) in a composition as a solution in sterile water, normal saline or another pharmaceutically acceptable carrier. The term “unit dosage form,” as used herein, refers to physically discrete units suitable as unitary dosages for human and animal subjects, each unit containing a predetermined quantity of compounds of the present invention calculated in an amount sufficient to produce the desired effect in association with a pharmaceutically acceptable diluent, carrier or vehicle. The specifications for the novel unit dosage forms of the present invention depend on the particular peptidomimetic compound employed and the effect to be achieved, and the pharmacodynamics associated with each compound in the host. Those of skill in the art will readily appreciate that dose levels can vary as a function of the specific compound, the nature of the delivery vehicle, and the like. Preferred dosages for a given compound or agent are readily determinable by those of skill in the art by a variety of means.

### Kits & Systems & Other Compositions

Also provided are kits and systems that find use in practicing embodiments of the methods, such as those described as described above. The term “system” as employed herein refers to a collection of two or more different active agents, present in a single or disparate composition, that are brought together for the purpose of practicing the subject methods. The term kit refers to a packaged active agent or agents. In some embodiments, the subject system or kit includes a dose of a subject compound (e.g., as described herein) and a dose of a second active agent (e.g., as described herein) in amounts effective to treat a subject for a disease or condition associated with the G6PD deficiency.

Also provided are preservative preparations and kits that find use as a preservative for cellular samples, e.g., erythrocyte cells, by improves preservation of erythrocyte storage by decreasing the degree of hemolysis of the cells over time. In some cases, the preparations provide for storage stability of the cellular sample. Preservative preparations are compositions that include a G6PD-modulating agent (e.g., as described herein) (for example one or more of the subject compounds), either alone or in the presence of one or more additional components, e.g., any convenient components that find use in stabilizing or storing cells. In some cases, the preservative preparation finds use in conjunction with a blood collection tube or a cell preservative tube. In some cases, the tube is suitable for evacuation to facilitate sample collection or transfer.

Kits and systems for practicing the subject methods may include one or more pharmaceutical formulations. As such, in certain embodiments the kits may include a single pharmaceutical composition, present as one or more unit dosages, where the composition may include one or more nucleoside compounds (e.g., as described herein). In some embodiments, the kit may include two or more separate pharmaceutical compositions, each containing a different active agent, at least one of which is a nucleoside compound (e.g., as described herein).

Also of interest are kits and systems finding use in the subject methods, e.g., as described above. Such kits and systems may include one or more components of the subject methods, e.g., antioxidant, cells, enzyme substrates, dyes, buffers, etc. The various kit components may be present in the containers, e.g., sterile containers, where the components may be present in the same or different containers.

In addition to the above-mentioned components, a subject kits may further include instructions for using the components of the kit, e.g., to practice the subject method. The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, Hard Disk Drive (HDD), portable flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

The following examples are offered by way of illustration and not by way of limitation.

### EXAMPLES

### Example 1

### Materials and Methods

### Cell Culture

Lymphocytes derived from a normal subject (HG 00866) and a G6PD-deficient subject carrying Canton variant in G6PD (HG 02367) were purchased from Coriell Institute and cultured in RPMI 1640 supplemented with 15% fetal bovine serum (FBS), 100 U/ml penicillin, and 100 μg/ml streptomycin. An SH-SY5Y neuroblastoma cell line was cultured in Dulbecco's Modification of Eagle's Medium/Ham's F-12 50/50 Mix supplemented with 10% FBS, 100 U/ml penicillin, and 100 μg/ml streptomycin. A G6PD-deficient subject-derived fibroblast cell line carrying Mediterranean variant and normal fibroblast cell line as control were purchased from Coriell Institute (GM 01152) and ThermoFisher Scientific (C0135C), respectively, and cultured in minimum essential medium supplemented with 15% FBS, 100 U/ml penicillin, and 100 μg/ml streptomycin. All the cell lines were maintained at 37° C. in a humidified incubator with an atmosphere of 5% of CO₂ and 95% air.

### Plasmid Construction and Site-Directed Mutagenesis

The gene encoding wild-type (WT) G6PD was inserted into the pET-28a vector, using NdeI and SalI restriction sites by polymerase chain reaction (PCR). Site-directed mutagenesis to generate G6PD variants was performed using appropriate primer sets according to the manufacturer's guidelines (Agilent). All constructs were verified by sequencing.

### Protein Expression and Purification

G6PD and its variants were expressed in E. coli C43 (DE3). When the culture density reached an OD600 of 0.5-0.6, 0.5 mM IPTG was added to induce the protein expression. The culture was allowed to grow for additional 4-5 hours at 37° C. and then harvested by centrifugation. The pellets were lysed by sonication in buffer containing 50 mM Tris (pH 7.4), 300 mM NaCl, 5% glycerol, 0.4 mM PMSF, 1 mg/ml lysozyme, 0.1% Triton X-100, and protease inhibitor cocktail (Sigma P8340). G6PD was then purified by incubating the supernatant with TALON Superflow resin equilibrated with 1 bed volume of equilibration buffer containing 50 mM Tris (pH 7.4), 300 mM NaCl, and 5 mM imidazole. The resin was washed with 5 bed volumes of wash buffer containing 50 mM Tris (pH 7.4), 300 mM NaCl, and 20 mM imidazole in a gravity-flow column and resuspended in 5 ml of equilibration buffer of gel filtration chromatography (50 mM Tris (pH 7.4), 150 mM NaCl, and 1 mM EDTA). 100 Units of bovine thrombin was added to the resin, which was followed by overnight incubation at 4° C. with gentle shaking. Tagless G6PD was eluted and applied onto HiLoad Superdex 200 pg gel filtration chromatography. Fractions containing G6PD were pooled and concentrated using 10 kDa MWCO membrane. The final concentration of G6PD was determined by Bradford method, and the protein was stored in 40% glycerol and 1 mM EDTA at −80° C.

### Enzyme Assay and Kinetic Measurements

Enzyme activity was measured by monitoring NADPH production, which was coupled with diaphorase converting resazurin to fluorescent resorufin (excitation at 565 nm and emission at 590 nm); fluorescent signal was thus proportional to G6PD activity. Assays were performed at 25° C. and run for 5 minutes in buffer containing 50 mM Tris (pH 7.4), 0.5 mM EDTA, 3.3 mM MgCl₂, 1 U/ml diaphorase, 0.1 mM resazurin. 10 ng of recombinant enzymes or 10 μg of cell lysates was used for the assay with 10 μM NADP⁺ (Sigma) as cofactor and 100 μM G6P (Sigma) as substrate. Steady-state kinetic parameters were obtained by varying concentrations of NADP⁺ (0-10 μM) with a constant concentration of G6P (100 μM) and similarly for G6P (0-100 μM) with a constant concentration of NADP⁺ (10 μM) with 10 ng of recombinant enzymes. Data analysis was performed using GraphPad Prism software v.6 (GraphPad Software, La Jolla, Calif. USA). Kinetic parameters were obtained by fitting the data to the Michaelis-Menten equation.

### High-Throughput Screening Assay

The diaphorase-coupled enzyme assay as described above was used to screen a library of compounds for small molecule activators. The compounds were added to Canton G6PD enzyme reaction mixture at a final concentration of 16.67 μM using Caliper Life Sciences Staccato system with a Twister II robot and a Sciclone ALH3000 (Caliper Life Sciences, Alameda, Calif. USA) integrated with a V&P Scientific pin tool, which was followed by incubation for 3 hours. Addition of G6P then initiated the reaction, which was run for 2.5 minutes. The fluorescent signals were recorded 4 times during the run using Molecular Devices AnalystGT (Molecular Devices, Sunnyvale, Calif. USA). Any compounds showing 30% activation of the enzyme were rescreened in a dose-dependent manner (0-30 μM, duplicate) to identify potential hits.

### Thermostability Assay

10 ng of recombinant enzymes was incubated at various temperatures ranging from 25−65° C. for 20 minutes, and the activity was measured as described above. After normalization of the data between 0 and 100, Boltzmann sigmoidal equation was used to calculate the T₁₁₂ value, the temperature at which the enzyme retains half of original activity.

### In Vitro Proteolysis Assay

200 ng of recombinant enzymes were incubated with 1 μL of 10 ng/μL chymotrypsin for 1 hour at room temperature. 100 μM of the compound was added to some reaction conditions. The following protein level was examined by Western blot.

### Overexpression of WT G6PD and Canton Variant in SH-SY5Y Cells

Prior to the cellular-based assays using SH-SH5Y cells, duration of overexpression of WT G6PD and Canton variant was examined by transfecting the cells seeded in a 12-well plate. The genes encoding human WT G6PD and Canton variant were first PCR-amplified, which was then inserted into pcDNA 3.1/myc-His C (ThermoFisher Scientific) using HindIII and XhoI. 0.5 μg of cDNA and 1.5 μg of lipofectamine (Invitrogen) were diluted in 50 μL of Opti-MEM medium respectively and incubated for 5 minutes. The diluted DNA was combined with diluted lipofectamine, which was followed by incubation for 20 minutes prior to the addition to cells.

The transfected cells were collected at different time points (up to 72 hours) and the overexpressed G6PD levels were examined by Western blot. The transfection was carried out in serum-starved cells. Once the duration of expression was confirmed, other cellular-based assays were performed.

### Cycloheximide Chase Assay

50,000 cells (SH-SH5Y cells or fibroblast cells) or 100,000 cells (lymphocytes) were seeded in a 12-well plate and incubated overnight. The cells were subjected to serum starvation (50-75%) for 48 hours and treated with 50 μg/ml of cycloheximide at different time points (0-48 hours). The cells were treated with the compound (1 μM) for 48 hours together with cycloheximide. Then the cells were collected in PBS containing protease inhibitor cocktail and 1% Triton X-100 and centrifuged at 14,000 rpm at 4° C. for 10 minutes. 10 μg of total protein was loaded onto the SDS-PAGE gel and the protein level was examined by Western blot.

### Glutathione (GSH) Measurement

Total glutathione level was measured using a Total Glutathione Quantification Kit (Dojindo), according to the manufacturer's instructions. The assay utilized DTNB (5,5′-dithio-bis-(2-nitrobenzoic acid)) that reacts with glutathione to produce 5-mercapto-2-nitrobenzoic acid, a yellow colored product. Cells were subjected to serum starvation (50-75%) for 48 hours to induce oxidative stress and treated with the compound for 24 hours before measurement. The absorbance was read at 412 nm.

### Cell Viability Assay

Cell viability was measured using a Cell-Counting Kit-8 (CCK-8, Dojindo), utilizing WST-8 [2-(2-methoxy-4-nitrophenyl)-3-(4-nitrophenyl)-5-(2,4-disulfophenyl)-2H-tetrazolium monosodium salt, according to the manufacturer's instructions. Cells were subjected to serum starvation for 48 hours before measurement. Cells in 100 μL of medium per well were treated with 10 μL of CCK-8 solution and incubated for 2 hours at 37° C. The absorbance was read at 450 nm. Viability of lymphocytes was measured by staining with a 0.4% solution of trypan blue in buffered isotonic salt solution (pH 7.2). The viability was calculated as the number of viable cells (non-stained by the dye) divided by the total number of cells within the grids on the hemacytometer. Cells were treated with 1 μM of the compound 24 hours before the measurement.

### Cellular Reactive Oxygen Species (ROS) Measurement

Cells in a 96-well plate were incubated with chloromethyl-2′,7′-dichlorodihydrofluorescein diacetate (CM-H2DCFDA) at a final concentration of 5 μM in HBSS (Hank's balanced salt solution) for 30 minutes at 37° C. After wash, cells were treated with Hoechst 33342 (1:10,000 dilution, Molecular Probes) to stain nuclei and incubated for another 10 minutes at 37° C. Cells were washed in HBSS, and the florescence was analyzed with excitation/emission at 485/525 nm. Cells were subjected to serum starvation (50-75%) for 48 hours to induce oxidative stress and treated with the compound for 24 hours before measurement. The signal was normalized to nuclei staining (excitation/emission at 350/470 nm).

### Native Gel Electrophoresis

200 ng of recombinant enzymes were incubated with the compound (varying in assays) for 10 minutes at room temperature. To prevent streaking and artifacts in native PAGE gel, the samples in a native state (no boiling of sample and no reducing agent in sample buffer) were electrophoresed by SDS-PAGE. Cross-linking assay was initiated by incubating 500 ng of recombinant enzymes in PBS with 0.1% of glutaraldehyde and different concentrations of NADP⁺ (0, 10, 100, 1000 μM) or MgCl2 (0, 1, 10, 100 mM). The mixture was incubated for 10 minutes at room temperature, which was followed by the addition of 100 mM Tris (pH 8.0) at a final concentration to terminate the reaction. The samples were electrophoresed as described above.

G6PD siRNA-Knockdown Assay

Endogenous G6PD was knocked down in each cell line as follows; 2 pmol of siRNA G6PD (Santa Cruz Biotechnology) and 0.5 μg lipofectamine (Invitrogen) were diluted in 10 μL of Opti-MEM medium respectively and incubated for 5 minutes. The diluted siRNA was combined with diluted lipofectamine, which was followed by additional incubation for 20 minutes prior to the addition to cells in a 96-well plate.

### Zebrafish Husbandry

Adult zebrafish (AB strain; 3-18 months old) were raised and embryos were obtained through natural mating and staged according to standard protocols. Adult tbx16b104/+ were obtained. All animal procedures were performed according to NIH guidelines. Embryos were raised in E3 medium at 28.5° C.

### Zebrafish Crispants

sgRNA against Exon 10 of g6pd was designed using CHOPCHOP and in vitro transcribed according to standard protocols. The gene-specific oligo sequence was: 5′-TAATACGACTCACTATAGAGAAGGGGAGGCAAAACTGGTTTTAGAGCTAGAAATAG CAAG-3′ (SEQ ID NO: //). sgRNA and Cas9 protein (NEB) were mixed and microinjected into one-cell-stage embryos. For each injected clutch, 10 individual embryos were isolated at 24 hpf for sequencing to confirm introduction of a CRISPR-mediated indel in exon 10.

### Zebrafish Compound Treatment

Embryos were dechorinated with pronase at 24 hours post fertilization (hpf) and treated with 50 μg/ml of chloroquine and/or AG1 by directly adding the compounds to the well. For ROS measurements, embryos were incubated with compounds for 2 hours, and then the ROS detecting reagent (CM-H₂DCFDA) was added at a final concentration of 500 ng/ml to the well for a total treatment time of 5 hours. One embryo was placed to each well of a black, opaque 96-well plate. The florescence was analyzed with excitation/emission at 485/525 nm. After ROS assays, embryos at about 32 hpf were pooled and lysed in buffer containing 50 mM Tris (pH 7.4), 150 mM NaCl, 1 mM EDTA, 0.1% NP-40, and protease inhibitor cocktail, which was followed by three cycles of freeze and thawing in liquid nitrogen. The lysate was centrifuged at 14,000 rpm at 4° C. for 15 minutes. Total protein concentration in the supernatants (total lysate) was determined by Bradford method. 50 μg of total lysate was loaded onto 10% SDS-PAGE gels and the protein level was examined by Western blot using anti-G6PD antibody (Abcam (G6PD: AB87230)). 10 μg of total lysate was used for enzymatic assay. 50 μg of the lysate was used to measure NADPH levels using a NADPH quantification kit (Biovision), according to the manufacturer's instructions.

### Zebrafish Imaging

Live embryos were anesthetized and mounted in 3% methylcellulose. Embryos were imaged with a Leica M205FA microscope equipped with a 1.0× Plan Apochromatic objective and a SPOT Flex camera or a Leica DM4500B compound microscope equipped with a QImaging Retiga-SRV camera. For hemoglobin staining, fixed embryos were stained with o-dianisidine as previously reported (10), cleared, mounted in 100% glycerol, and imaged with a Leica M205FA microscope equipped with a 1.0× Plan Apochromatic objective and a SPOT Flex camera. All images were captured using SPOT or MetaMorph imaging software (Diagnostic Imaging Inc.) and processed in Photoshop (Adobe). Adjustments were limited to brightness levels and cropping. Analysis was carried out by an observer blinded to the experimental conditions.

### Blood Sample Assay

De-identified blood samples were obtained from the Stanford Blood Center. Erythrocytes were collected by filtering the samples through a cellulose slurry to remove platelets and leukocytes and then washed with saline. G6PD activity was measured (Beutler E. Red cell metabolism: A manual of biochemical methods (3rd edition). Grune & Stratton (1984)). The activity of all the samples used in this study was in a normal range (5-9 U/g Hb), suggesting that the subjects have WT G6PD. A 5% erythrocyte suspension was pre-incubated with 1-5 μM AG1 at 4° C. overnight, which was followed by treatment with (or without) either 1 mM chloroquine (CQ) or 1 mM diamide for 3-4 hours at 37° C. (for hemolysis assay with chloroquine, the mixture was incubated under light). Then centrifugation at 1,000 rpm for 5 minutes was followed. Hemoglobin release in the supernatant was monitored by measuring absorbance at 540 nm. Saline was used as a negative control (0% hemolysis) and a sample treated with 0.1% Triton X-100 was used as a positive control (100% hemolysis). For ROS measurement, erythrocyte mixture was washed with saline by centrifugation after treatment and incubated with chloromethyl-2′,7′-dichlorodihydrofluorescein diacetate (CM-H₂DCFDA) at a final concentration of 5 μM in saline at 37° C. for 15 minutes. After wash, the samples were lysed with 0.1% Triton X-100 (final concentration), and the florescence was analyzed with excitation/emission at 485/525 nm. GSH measurement was determined using a Cayman glutathione assay kit (Cayman Chemicals, 703002). Briefly, 50 μl of diluted erythrocyte lysate samples were mixed with 150 μl of assay reagents including glutathione reductase, 5,5′-dithio-bis-(2-nitrobenzoic acid) (DTNB) and NADPH, which was followed by incubation for 25 minutes at room temperature. The absorbance was read at 412 nm. For storage assay, a 5% erythrocyte suspension was stored at 4° C. with and without 1 μM AG1, and hemolysis and G6PD activity were monitored every week for 28 days to examine whether AG1 improves preservation of erythrocytes over time. Protein leakage was also examined by measuring the absorbance of the supernatant of samples at 280 nm. The samples were re-treated with AG1 every week.

### Statistical Analyses

Most assays were repeated at least in three independent experiments. The in vitro and cellular data are presented as mean±standard error of the mean (SEM).

Statistical differences between mean values were calculated by Student's t-test using GraphPad Prism software. In assays with human erythrocytes, each sample was utilized as its own control, and assay parameters were compared before and after treatment; thus, randomization was not needed. For all zebrafish experiments, at least two breeding tanks, each containing 3-4 males and 4-5 females from separate stocks were set up to generate embryos. Embryos from each tank were randomly distributed across tested conditions. No statistical methods were used to determine sample size per condition. For phenotypic analysis, raw counts for each condition were used for chi-square analysis or Fisher's exact test based on expected values. For all phenotypic analysis, the scorer was blinded to treatment conditions. For ROS assays, the normality of each distribution was assessed and determined to be non-normal. A Kruskal-Wallis test with a Dunn's secondary test was used to determine differences between all conditions. P values were corrected for multiple comparisons testing. P values and number of embryos are indicated within the figure legends, and P<0.05 was considered statistically significant.

### Example 2

Correcting Glucose-6-Phosphate Dehydrogenase 1 (G6PD) Deficiency with a Small Molecule Activator

From single-cell organisms to eukaryotes, the use of oxygen to generate ATP produces damaging oxygen free radical products. These are counteracted by cellular anti-oxidants, such as reduced glutathione (GSH). GSH, which provides the cellular first line of defense against oxidant injury, is maintained by NADPH generated mainly via the pentose phosphate pathway and its rate-limiting enzyme, glucose-6-phosphate dehydrogenase (G6PD; FIG. 1A). Accordingly, missense DNA mutations that impair G6PD activity or stability result in increased oxidative stress and a spectrum of disease phenotypes featuring, most commonly, hemolytic anemia, and collectively called G6PD deficiency (A. Minucci et al., Glucose-6-phosphate dehydrogenase (G6PD) mutations database: review of the “old” and update of the new mutations. Blood cells, molecules & diseases 48, 154-165 (2012)). Although G6PD is a ubiquitous enzyme expressed in all tissues, it is particularly essential in preserving the integrity of erythrocytes because, lacking mitochondria, they have no other sources of anti-oxidants to protect against oxidative stress. G6PD deficiency afflicts an estimated 400 million individuals worldwide. Symptoms can be triggered by oxidative stress induced by certain foods, medications, or infection. G6PD deficiency can be life-threatening, especially in newborns, leading to bilirubin-induced neurological injury and bilirubin encephalopathy (kernicterus) and even to death.

G6PD is functionally active as a dimer or a tetramer (P. Cohen, M. A. Rosemeyer, Subunit interactions of glucose-6-phosphate dehydrogenase from human erythrocytes. European journal of biochemistry 8, 8-15 (1969)). Each monomer has a catalytic NADP⁺-binding domain and β+α domain, containing an additional binding site for NADP⁺ that structurally stabilizes the enzyme (FIG. 1B). The G6P binding site is located between these two domains (FIG. 1B). The majority of the variants that cause severe or mild deficiency are primarily located in those functional regions of the enzyme, disturbing the enzyme's activity and stability (A. D. Cunningham, A. Colavin, K. C. Huang, D. Mochly-Rosen, Coupling between Protein Stability and Catalytic Activity Determines Pathogenicity of G6PD Variants. Cell reports 18, 2592-2599 (2017)).

Efforts to understand the biochemical mechanism of the Canton variant (R459L), located in the β+α domain (FIG. 1B) were undertaken. Canton G6PD is prevalent in China and Southeast Asia [50-60% of the variants (15, 16)], causing severe deficiency. Recombinant Canton G6PD enzyme showed only 18% (160 μmol min⁻¹ mg⁻¹) of normal G6PD activity (884 μmol min⁻¹ mg⁻¹) with lower KM for both NADP⁺ and G6P (FIG. 1C). This is consistent with the biochemical characteristics analyzed using blood samples derived from subjects carrying the Canton variant (N. Saha, S. H. Hong, P. S. Low, J. S. Tay, Biochemical 269 characteristics of four common molecular variants in glucose-6-phosphate dehydrogenase-deficient Chinese in Singapore. Human heredity 45, 253-257 (1995)). When crosslinked by 0.1% of glutaraldehyde, Canton G6PD displayed impaired ability to form tetramers in the presence of increased concentrations of the cofactors that facilitate tetramer formation, NADP⁺ or MgCl₂, relative to the wild-type (WT) G6PD. This reduced oligomerization state of the Canton G6PD may contribute to its reduced enzymatic activity relative to WT enzyme. Furthermore, the Canton variant was less thermostable; its T_{1/2}, the temperature at which the enzyme retains half of its catalytic activity was 4.5 degrees lower for Canton G6PD relative to normal WT enzyme (FIG. 1D). Canton G6PD was also more susceptible to degradation by chymotrypsin relative to the WT enzyme, which corresponded with significantly decreased enzyme activity (FIG. 1E). This suggests that Canton G6PD may undergo higher conformational fluctuation and leading to reduced thermostability and a greater accessibility to proteases. The Canton variant was also less stable than WT G6PD in lymphocytes derived from a subject who carries a Canton variant in G6PD; 24 hours after cycloheximide treatment (50 μg/ml) to inhibit de novo protein biosynthesis, the level of Canton variant protein was ˜33% lower than WT G6PD (FIG. 1F). G6PD activity in lysates of cells carrying the Canton variant was ˜90% lower than G6PD activity in normal lymphocytes (FIG. 1G), which coincided with low level of total GSH and increased levels of reactive oxygen species (ROS) (FIG. 1H and FIG. 1I).

Moreover, the viability of the lymphocytes with Canton variant 82 was ˜50% lower relative to normal lymphocytes when cultured in the absence of serum for 48 hours to induce oxidative stress (FIG. 1J). The same results were observed in SH-SY5Y neuronal cells transiently expressing WT G6PD or the Canton variant (His-tagged). Canton G6PD protein levels dropped by 50% within 24 hours of cycloheximide treatment, as compared to 20% decrease of WT G6PD. SH-SY5Y cells expressing the Canton variant also showed lower enzymatic activity in the lysates and a lower level of GSH and knockdown of G6PD by siRNA suppressed cell viability, recapitulating G6PD deficiency.

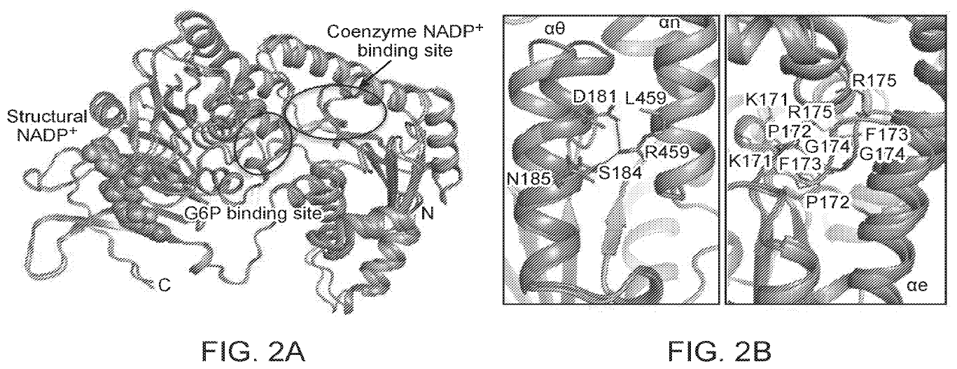

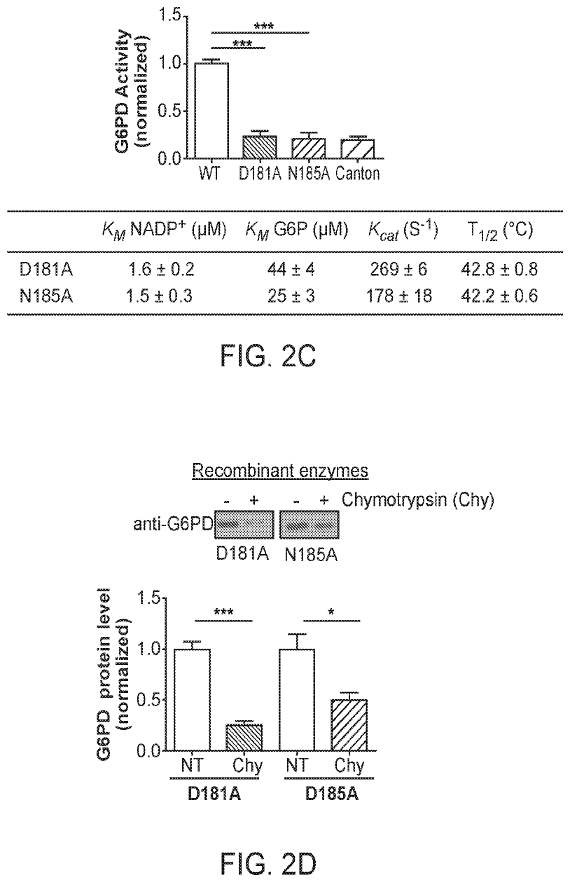

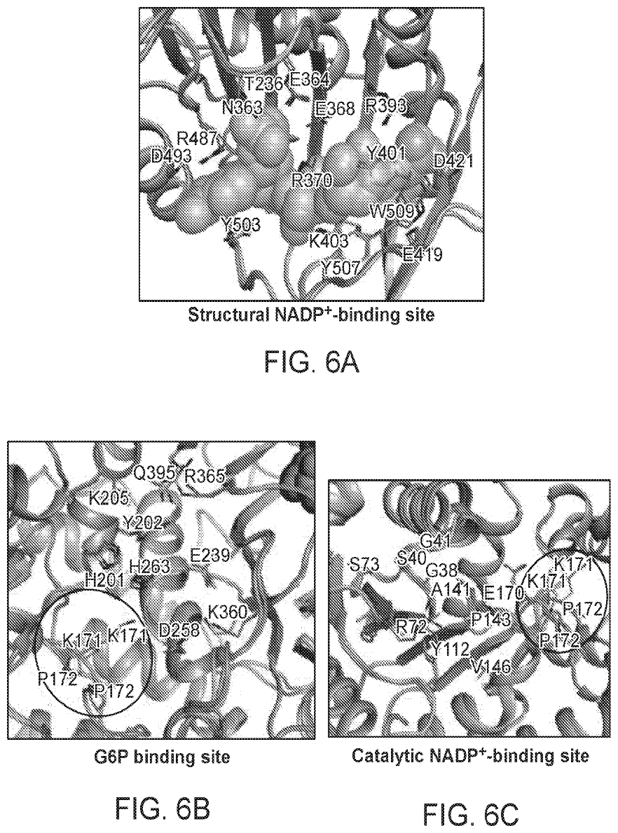

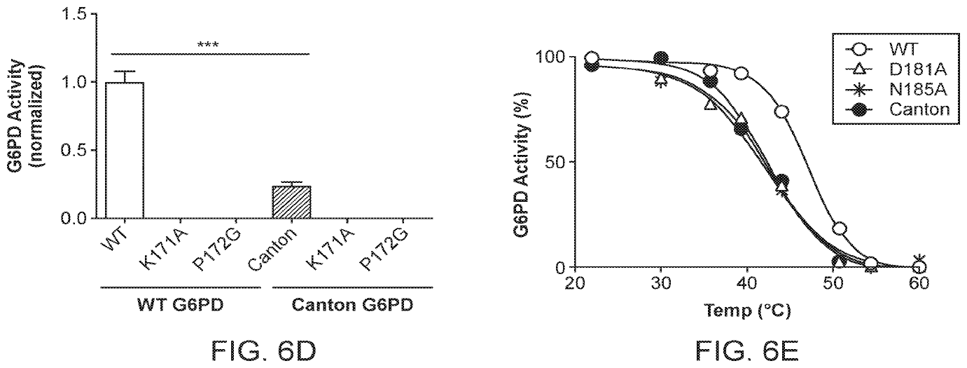

The structural mechanism of Canton G6PD was next investigated to understand its reduced metabolic function, for which we examined the crystal structures of WT and Canton G6PD at 1.9 Å and 2.6 Å resolution, respectively. WT G6PD was crystallized in the F222 space group containing one molecule in the asymmetric unit of the crystal. Canton G6PD crystals belonged to the P212121 space group with 8 molecules of G6PD protein in the asymmetric unit of the crystal. Although no NADP⁺ was added to protein solution prior to the crystallization, both crystal structures contained NADP⁺ at the second NADP⁺-binding site (structural NADP⁺-binding site) (FIG. 6A). The overall conformations of WT and Canton G6PD were very similar, as indicated by a root-mean-square deviation of 0.6 Å for the superimposition of Cα atoms (FIG. 2A). However, a loose helical interaction was noticed between the helix (αn) containing the Canton variant (R459L) and an adjacent helix (αe) (FIG. 2B, left panel), which was not described in previously reported structures (Au et al., Structure 8, 293-303 (2000)). In WT G6PD, R459 forms electrostatic and hydrogen bond interactions with D181 and N185 on the adjacent helix (αe), whereas Canton mutation does not, resulting in loosened inter-helical interactions and displacements of the helix (αe) and a proceeding loop consisting of K171, P172, F173, G174, and R175, a few amino acids away from R459-interacting residues on the helix (αe) (FIG. 2B, right panel). In particular, K171 and P172 are the key residues involved in positioning of G6P and NADP⁺ in their binding pockets.

Thus, the loose inter-helical interaction in the Canton variant is likely to be a major driving force for positioning the loop and thus changing the orientations of these residues. In 7 out of 8 molecules in the asymmetric unit of the Canton variant structure, P172 was observed in the trans conformation and accordingly the side chain of K171 was oriented away from catalytic NADP⁺ and G6P binding pockets (FIG. 2B, right panel, FIGS. 6B and 6C). Mutations of K171A and P172G completely abolished catalytic activity, indicating the importance of these residues in catalysis (FIG. 6D).

To determine whether the inter-helical interactions between R459 on the an helix and its interacting residues on the αe helix are essential for enzyme activity and stability, the interacting residues were mutated to alanine in WT G6PD and found that these point-mutated proteins are biochemically similar to the Canton variant; they exhibited about 20% of normal activity with lower KM for both NADP⁺ and G6P and lower T½ values (FIG. 2C). These mutants were also more susceptible to proteolytic digestion by chymotrypsin, relative to WT G6PD (compare FIG. 2D to FIG. 1E). Taken together, these data support the importance of the interhelical interaction between R459 and D181 and N185 for catalytic activity and stability. Indeed, human mutations located around this helical interaction site, such as P172S (Class I; <10% enzyme activity), F173L (Class II; <10% enzyme activity), and D181V (Class III; 10-60% enzyme activity) cause moderate to severe G6PD deficiency, likely because they are predicted to undergo similar conformational changes based on our observation.

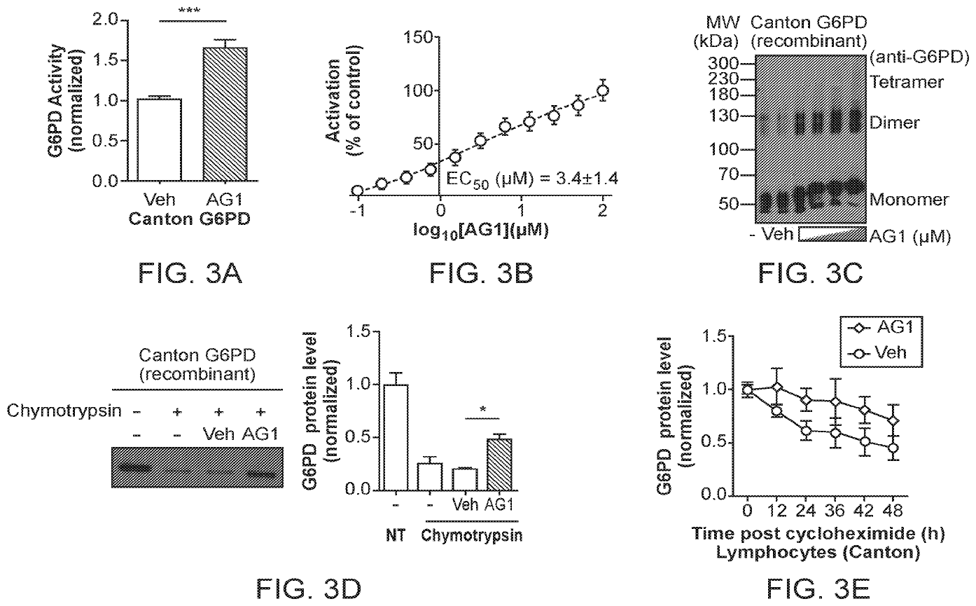

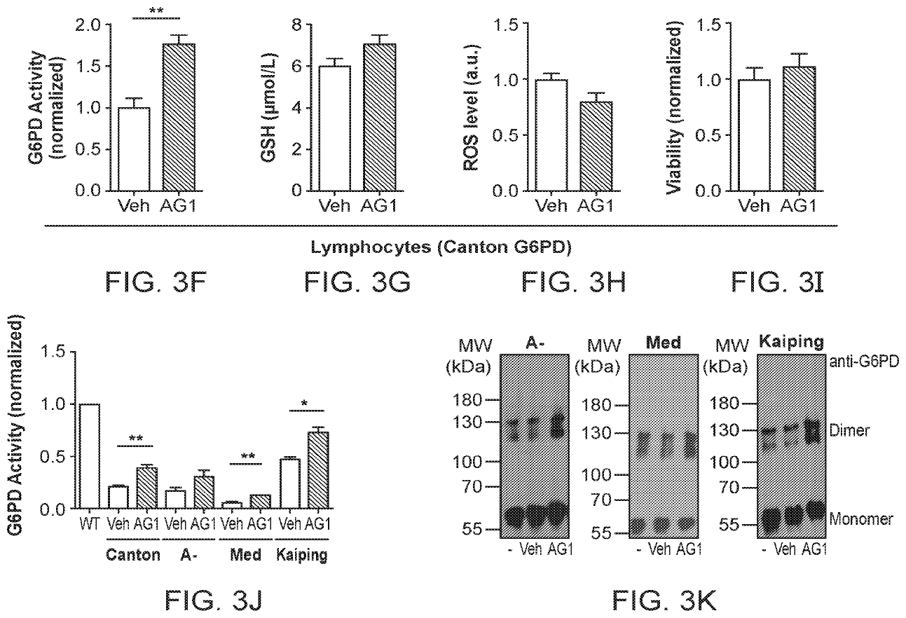

These biochemical and structural studies indicate that restoring (correcting) G6PD activity of G6PD variants with a pharmacological agent can provide a therapeutic approach to reduce the risk of pathologies implicated in patients with G6PD deficiency. To this end, screening for agonists of G6PD (AGs) was performed using the recombinant Canton G6PD enzyme by a high throughput screen. Several agonist compounds were identified including, 2-[2-(1H-indol-3-yl)ethylamino]ethanethiol (AG1, Mr=220.34). It was confirmed that AG1 increased the activity of Canton G6PD up to 1.7-fold with EC50≈3 μM and WT G6PD by about 20% over basal activity (FIGS. 3A and B). Although AG1 was a mild activator, it changed the kinetic parameters of Canton G6PD, indicating that AG1 can facilitate improved binding of NADP⁺ and G6P to the enzyme (Table 5).

TABLE 5

| Canton G6PD | K_{M} NADP⁺ (μM) | K_{M} G6P (μM) | k_{cal} (S⁻¹) |
|---|---|---|---|
| Veh | 1.2 ± 0.2 | 36 ± 0.8 | 193 ± 2 |
| AG1 | 2.3 ± 0.2 | 61 ± 0.6 | 296 ± 1 |

AG1 also promoted formation of active dimers, as determined by native gel electrophoresis (FIG. 3C). An increase in molecular weight of monomeric G6PD might be due to either some modification by AG1 or an equilibrium shift toward dimeric states. AG1 had no effect on the dimerization or activity of several other NAD or NADP⁺-dependent dimeric or tetrameric enzymes, including 6-phosphogluconate dehydrogenase (6PGD), glyceraldehyde 3-phosphate dehydrogenase (GAPDH), aldehyde dehydrogenase 2 (ALDH2), and aldehyde dehydrogenase 3A1 (ALDH3A1). Whereas 1 μM of AG1 increased the viability of SH-SY5Y cells by 20%, it had no effect when G6PD was knocked down by siRNA, supporting the selectivity of AG1 toward G6PD. AG1 also reduced the susceptibility of Canton G6PD to proteolysis; in the presence of AG1, 50% of the protein remained, relative to 25% in its absence (FIG. 3D). The stability of Canton G6PD was improved by AG1 treatment of lymphocytes; in 24 hours following cycloheximide treatment, the level of Canton protein was 48% higher with AG1 treatment (1 μM) relative to the level with vehicle treatment (FIG. 3F). The enzymatic activity in lymphocyte lysates carrying the Canton variant (˜10% of normal activity) was enhanced by 78% 150 in the presence of AG1 (FIG. 3F). The level of GSH was slightly increased after treatment with AG1 for 24 hours, which coincided with decreased ROS level and improved viability (FIG. 3G to 3I) in Canton variant carrying lymphocytes. AG1 treatment also increased the stability of Canton G6PD in SH-SY5Y cells. There was also a mild increase in G6PD activity and viability of these cells. Finally, as predicted, Canton variant mimicking mutations that were generated (FIGS. 2C and 2D), D181A and N185A, were similarly affected by AG1, as measured by increased activity and proteolytic stability, suggesting that AG1 may correct a structural defect.

Because G6PD exhibits cooperative folding and AG1 increased G6PD dimerization, the possibility that binding of AG1 to stabilizing site(s) of the enzyme could correct other point mutation-containing G6PD variants outside the inter-helical interaction sites around the Canton mutation was examined. A- (V68M & N126D), Mediterranean (S188F), and Kaiping (R463H) G6PD, the three most common human variants in non-overlapping regions of 164 the world causing mild to severe deficiency (Africa, Mediterranean and Southeast Asia, respectively) were focused on. AG1 activated all these variants by up to 2-fold and promoted their dimerization (FIGS. 3J and 3K). AG1 also stabilized G6PD in fibroblast cells derived from a subject who carries Mediterranean variant in G6PD with cycloheximide treatment. The lysate of human fibroblasts with the Mediterranean variant had only 22% activity of control human fibroblasts, but AG1 increased that activity by 50%. The fibroblasts with the Mediterranean variant were also less viable under stress conditions relative to fibroblasts from control subject, but AG1 improved their viability by 22%. When Mediterranean G6PD expression was knocked down by siRNA, the viability was further dropped by 23%, and AG1 did not rescue it, indicating the selectivity of the 173 effect of AG1 for G6PD. Taken together, these data suggest that by increasing the dimerization state of G6PD and enzyme activity, AG1 represents a lead compound for a drug to treat not only Canton-like mutations, but also some of the other most common G6PD deficiencies in humans.

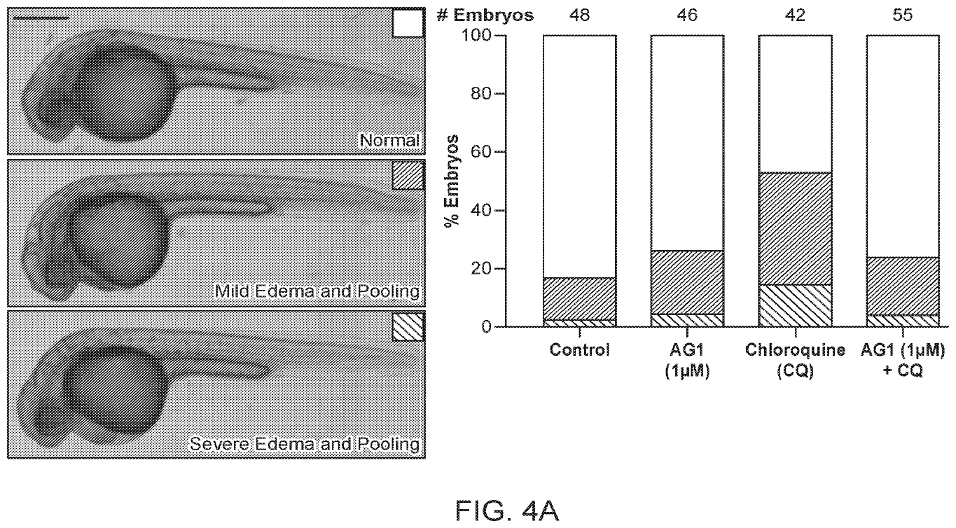

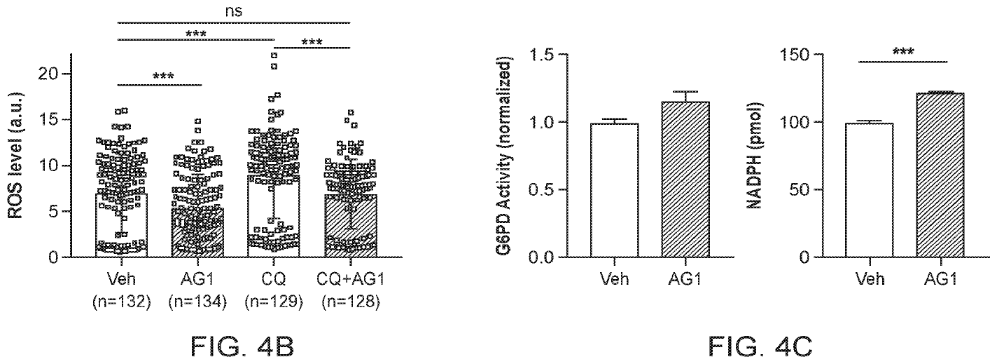

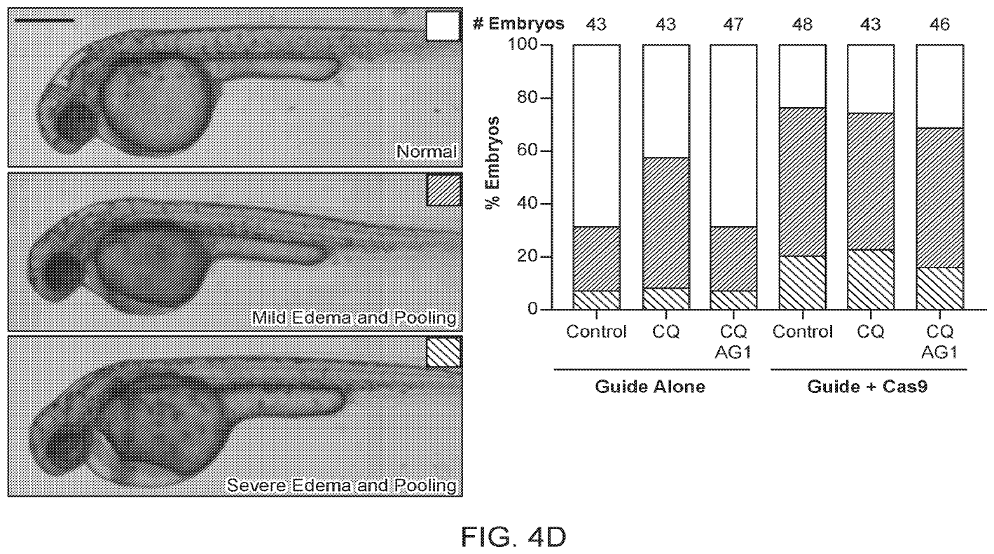

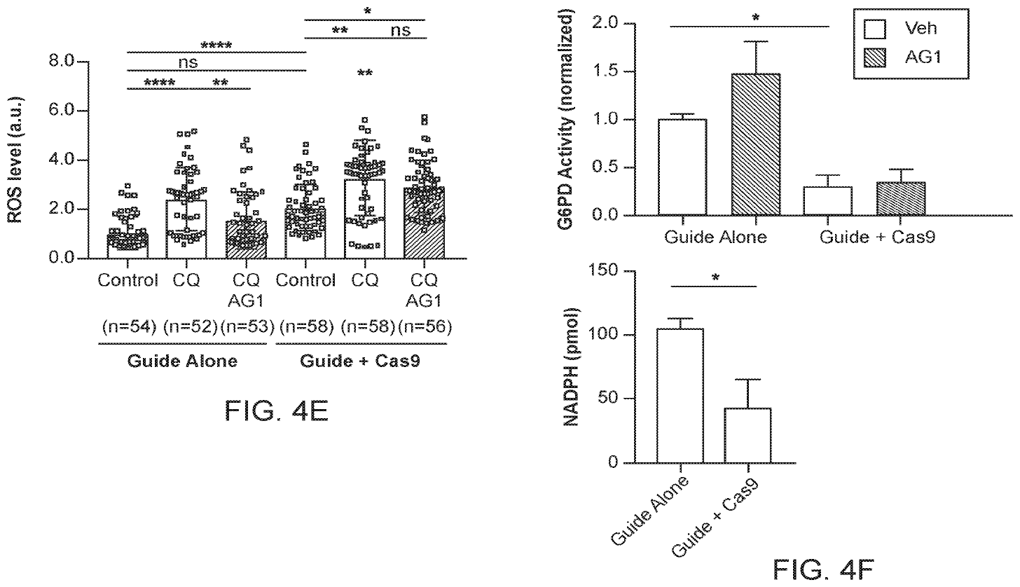

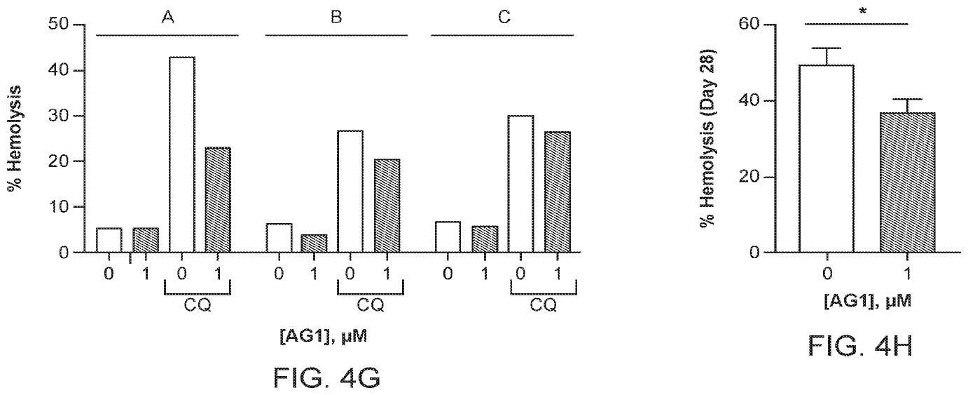

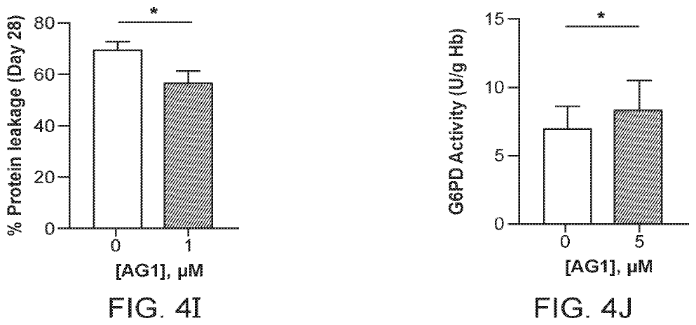

The effect of AG1 in vivo were also examined. A morpholino-based G6PD-deficient zebrafish model was used, in which exposure to pro oxidants results in cardiac edema and a brisk hemolysis (Patrinostro, M. L. Carter, A. C. Kramer, T. C. Lund, A model of glucose-6-phosphate dehydrogenase deficiency in the zebrafish. Experimental hematology 41, 697-710 e692 (2013)). Using zebrafish embryos, it was first determined whether AG1 affects their normal development. Embryos, which were treated with AG1 at 24 hours post fertilization (hpf) and phenotypically scored at 32 or 48 hpf, developed normally at concentrations<10 μM of AG1 (FIG. 4A), indicating that AG1 is not toxic to developing zebrafish embryos. The anti-malarial drug, chloroquine, a common trigger for crisis in G6PD-deficient humans, was used to induce an oxidative challenge in the zebrafish embryos and found that chloroquine (50 μg/ml) treatment at 24 hpf led to pericardial edema and increased ROS levels (FIGS. 4A and 4B). Embryos were stained to determine whether increased ROS results in a decrease in circulating erythrocytes, but hemoglobin staining did not significantly change upon chloroquine treatment. AG1 reduced ROS levels, resulting in less embryos exhibiting pericardial edema (FIGS. 4A and 4B). A slight increase in G6PD activity and a significant increase in NADPH levels were also observed in lysates of pooled AG1-treated embryos (FIG. 4C). As expected, the attenuation of pericardial edema was specific to G6PD deficiency, as pericardial edema due to mesoderm defects in tbx16 mutants was not corrected by AG1 treatment.

To confirm the specificity of AG1 in vivo, CRISPR-Cas9 was used to generate loss-of-function F0 embryos (crispants). g6pd crispant embryos had a lower G6PD level, a 51% higher level of ROS, a 67% lower level of G6PD activity and 196 a 58% lower NADPH level, and an increased pericardial edema (FIG. 4D to 4F). Treatment with 1 μM AG1 did not significantly affect these parameters in the g6pd crispants (FIG. 4D to 4F). Note also that there was a slight increase in the number of crispant embryos exhibiting reduced hemoglobin staining.

In humans, in addition to the role of G6PD in preventing hemolysis (erythrocyte lysis), the antioxidant property of G6PD may relate to development of a variety of other pathologies, including kidney injury, heart failure and cataract, suggesting that G6PD deficiency can be an under-estimated risk factor for multiple human pathologies. Because AG1 increased the impaired activity of several common G6PD variants, this study suggests that a single pharmacological agent, such as AG1, can provide treatment for several major G6PD enzymopathies. Such an agent may also help prevent or reduce the sequelae of G6PD deficiency and/or synergize with other palliative treatments such as illumination for kernicterus. Many other pathologies associated with G6PD deficiency may be affected by such treatment as well. Treatment with AGs may also be beneficial to G6PD-deficient populations in developing countries, in which the use of hemolytic crisis-triggering drugs, like the anti-malarial drug (primaquine and chloroquine), is still common. AG1-like drugs will be useful for subjects with WT G6PD with other diseases associated with increased oxidative stress. Human studies demonstrate that clinical pathology related to G6PD deficiency, at least as reflected by hemolytic crisis, occurs in subjects who carry a variant with <60% activity relative to the normal (WT) variant (Glucose-6-phosphate dehydrogenase deficiency. WHO Working Group. Bulletin of the World Health Organization 67, 601-611 (1989)). Therefore, an optimal AG compound could be improved to increase the catalytic activity in G6PD-deficient subjects to at least 60% of normal. The subject compounds can find use treating G6PD-deficient subjects.

### Example 3

### AG1 Reduces Hemolysis of Human Erythrocytes

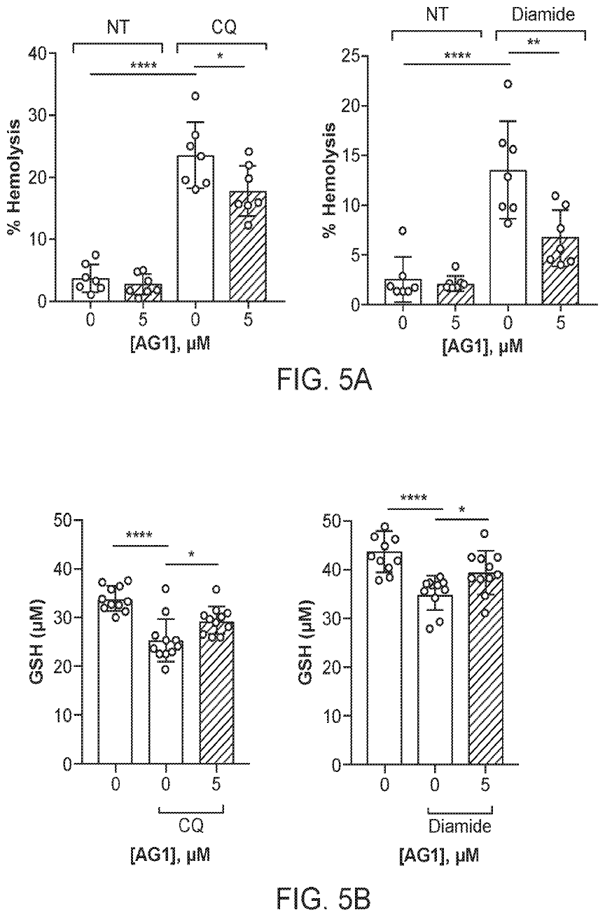

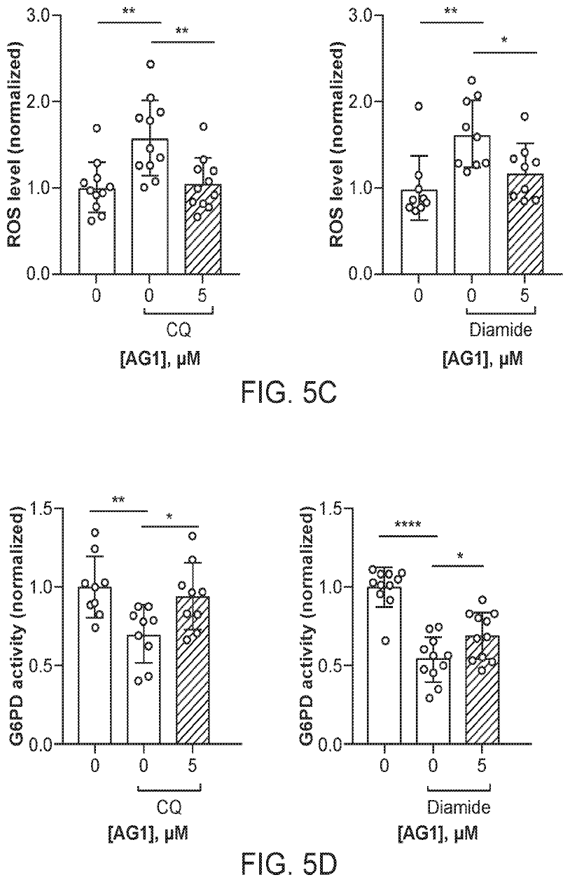

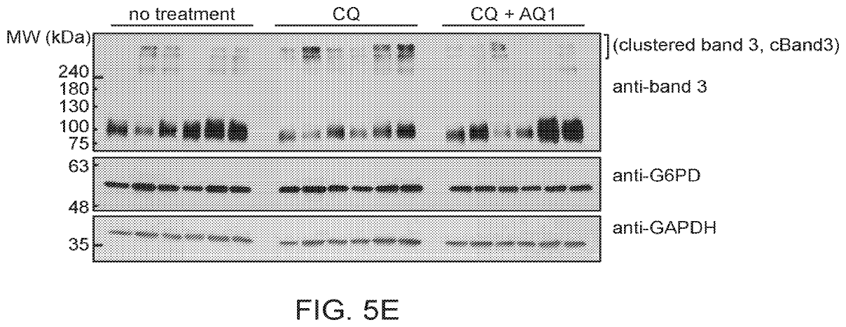

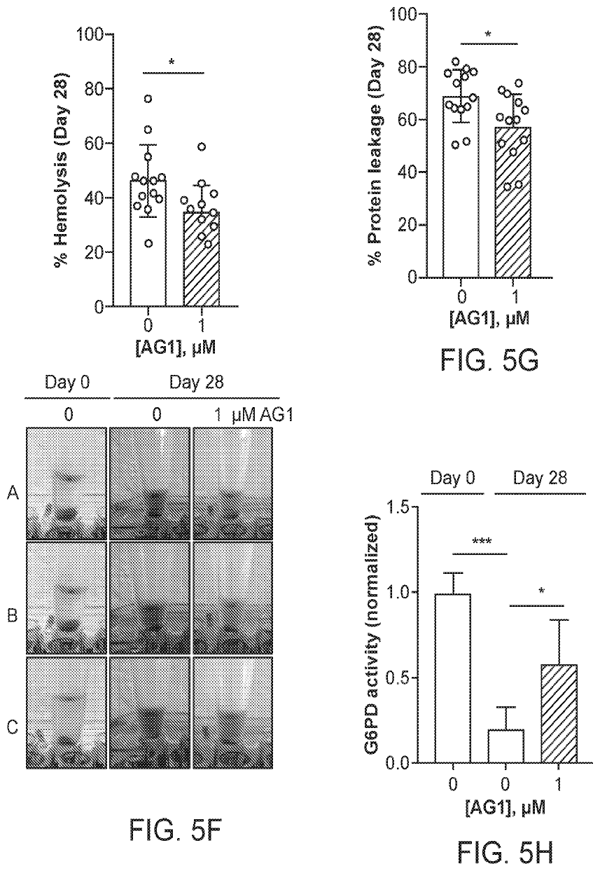

It was next determined whether AG1 protects erythrocytes from oxidative stress. A preliminary study using human erythrocytes from seven healthy subjects showed that AG1 (5 μM) reduces the extent of hemolysis, when erythrocyte suspension (5%) was exposed to either 1 mM chloroquine (CQ) or diamide (a GSH oxidant), suggesting anti-hemolytic potential of AG1 (FIG. 5A). In support of this, AG1 increased GSH levels and reduced ROS levels together with increased G6PD activity under these drug-induced oxidative stress (FIG. 5B, 5C, 5D). Oxidative stress impairs erythrocyte membrane integrity through initial oxidation of hemoglobin, leading to the precipitation of Heinz bodies and band 3 (a major erythrocyte membrane protein) clustering; thus, band 3 clustering serves as an essential molecular marker of erythrocyte removal (Shimo H, et al. Particle Simulation of Oxidation Induced Band 3 Clustering in Human Erythrocytes. PLoS Comput Biol 11, e1004210 (2015)). Using erythrocytes isolated from 9-11 individual whole blood samples, it was confirmed that band 3 protein is aggregated with either chloroquine (CQ) or diamide treatment, which was alleviated by AG1 treatment (FIG. 5E), suggesting that AG1 contributes to stabilizing erythrocyte membranes. Red blood cell transfusion is commonly used clinical therapy, but structural and functional changes in erythrocytes during storage, collectively referred to as storage lesion, remain a concern in transfusion practice. Storage under conventional conditions is considered as oxidative stress for erythrocytes, as evidenced by increase in ROS over time and accumulation of oxidative biomarkers. Thus, it was determined whether AG1 can improve preservation during refrigerated storage by monitoring the degree of hemolysis over 28 days. AG1 (1 μM) reduces hemolysis over time by an average of 12% at day 28 (FIG. 5F). Accordingly, the protein leakage from the treated erythrocytes was decreased as well (FIG. 5G), which corresponded with increased G6PD activity (FIG. 5H). These data suggest that AG1-like compounds can serve as a novel preservative for prolonged storage of erythrocytes and impact to a broader population as well as G6PD-deficient patients.

### Example 4

### G6PD Protein Crystallization

Crystals of WT G6PD grew in sitting drops containing 20% w/v PEG 3350, 0.2M potassium formate, pH 7.3. Suramin (G6PD inhibitor) was added to the protein solution (final concentration in the drop was 0.5 mM) prior to the crystallization. Canton G6PD was crystallized in sitting drops containing 20% w/v PEG 3350, 0.2M ammonium citrate tribasic, pH 7.0. AG1 compound dissolved in 30% DMSO was added to the protein prior to the crystallization to reach final concentration of 0.5 mM in the drop. None of the Suramin or AG1 compound was visible in the electron density map of WT G6PD and Canton G6PD; however, they significantly improved diffracting quality of the crystals. X-ray diffraction data of WT G6PD and Canton G6PD were collected at 100 K at beamline 12-2 of Stanford Synchrotron Radiation Light Source (SSRL) and beamline 5.0.2 of Advanced Light Source (ALS), respectively. A solution of 20% glycerol was used as cryo-protectant. Crystals of WT and Canton G6PD diffracted to 1.9 Å and 2.6 Å resolution, respectively. The data were processed using iMOSFLM, and further analysis of the data by POINTLESS indicated space group of F222 and P212121 for WT G6PD and Canton G6PD crystals, respectively. WT G6PD structure was solved using molecular replacement with a monomer G6PD structure from PDB: 2BHL used as a search model in MOLREP. Canton G6PD structure was solved using the WT G6PD structure that was already solved in this study. Molecular models were further built in Coot and refined using REFMAC5. The atomic coordinates and structure factors are deposited in the PDB database under accession codes PDB: 5VFL for WT G6PD and PDB: 5VG5 for Canton G6PD. All structure figures were prepared in PyMOL.

### Compound Design

In the Canton variant (R459L) of G6PD, a structural perturbation was identified by X-ray crystallography, which propagates into the active site region of the enzyme and lowers the catalytic efficiency. Using this enzyme, screening over 100,000 molecules for activators (chaperones) identified a potential molecule (referred to as AG1 hereafter) that activated the enzyme by at least 1.7-fold and significantly increased the enzyme's stability in cells. AG1 activated other common G6PD mutant enzymes as well, suggesting that it can be a general treatment for G6PD deficiency. Furthermore, AG1 alleviated oxidative stress-induced phenotypes in zebrafish recapitulating G6PD deficiency by CRISPR/Cas 9 system. The studies described herein suggest that a single pharmacological agent can provide a sufficient increase in functions of several G6PD variants to alleviate the clinical problems associated.

FIG. 7A illustrates an expanded view of the X-ray crystal structure of the Canton variant (R459L) of G6PD focused at the G6PD dimer interface and a proposed binding site of G6PD-modulating compounds. The cofactor NADP⁺ (also referred to as structural NADP⁺) is shown as black sticks.

The dimer interface near the structural NADP⁺ binding site has an array of residues that complement the subject compounds (e.g., compound 1, FIG. 7B). First, tryptophan 509 is pi-stacking with the NADP^{f} pyridinium ion and is only 5 angstroms away from aspartate 421. Compound contains an indole group 6 angstroms away from a cationic nitrogen. These groups are reflected about the dimer interface of G6PD which places the two 421 residues at an 8 angstrom distance. This distance is close to the 7 angstrom distance of the linker portion of the ligand (FIG. 8).

FIG. 9A illustrates two views of opposite faces of a space filing representation of the X-ray crystal structure of the Canton variant (R459L) of G6PD. Residues were mutated on either side of the dimer interface. The top panel illustrates various mutations (red, blue) located at the compound binding site. The top panel illustrates the location of various mutations (green, yellow and cyan) at the opposite face of the G6PD enzyme.

FIG. 9B shows a graph indicating the effect of various point mutations of the Canton variant of G6PD have on the G6PD-activity of exemplary compound AR3-069. The colored bars of the graph correspond to the mutations depicted in the structure of FIG. 3A. The Residues only at the putative binding site affect binding of the subject compounds whereas there was no effect on AC₅₀ (also EC₅₀) on the other side.

A co-crystal structure of exemplary compound AG1 and G6PD was obtained using the methods described here, which confirms the analysis described herein. The crystal structure of AG1 in complex with WT G6PD reveals a binding mode consistent with the hypothesis developed through mutagenesis experiments and docking. In the x-ray structure, the ligand is located at the dimer interface between the two structural NADP⁺ molecules. The indoles of AG1 provide pi-stacking interactions with the structural NADP⁺ pyridinium ions in a similar fashion to W509 in x-ray structures without ligand. The cationic amino groups are in close proximity to D421/E419 on either monomer separated by an average distance of approximately 8 angstroms. Linkers that place two cationic amines in close proximity to these anionic residues, while still allowing for pi-stacking to the structural NADP⁺ of both monomers, provide for stabilization and activation of dimeric G6PD. The bound structure of AG1 is in excellent agreement with the SAR observed with this class of compounds.

### Compound Activity

Based on preliminary results with compound AG1, a variety of compounds of interest were prepared and assessed in G6PD enzyme activation assays, according to the methods described herein.

TABLE 6
Canton G6PD Enzyme Activity of Compounds of interest

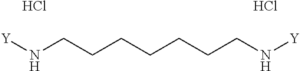

| | | Enzyme |
|---|---|---|
| Compound | Y | Activity^{a} (%) |
| AR3-118 | 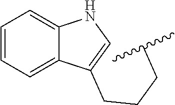 [SMILES: *CCCc1c[nH]c2ccccc12] | 113 |
| AR3-100 | 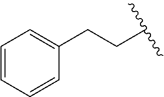 [SMILES: *CCc1ccccc1] | 132 |
| AR3-109 | 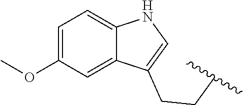 [SMILES: *CCc1c[nH]c2ccc(OC)cc12] | 98 |

(^{a})activation of canton G6PD with 100 μM compound relative to DMSO control

TABLE 7
G6PD enzyme activation activities of compounds of interest measured
using 100 uM compound on wt G6PD obtained from a blood sample

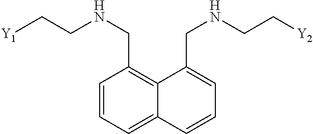

| | | Enzyme | |
|---|---|---|---|
| | | activity | Compound |
| Y₁ | Y₂ | (%) | |
| | | 127% | AR3-069 |
| | | | (AG1) |
| 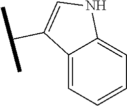 | 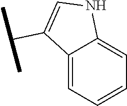 | 118% | AR4-061 |
| 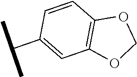 | 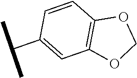 | 154% | AR4-078 |
| 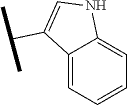 | 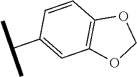 | 137% | AR4-134 |
| 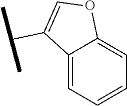 | 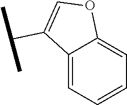 | 153% | AR5-005 |
| 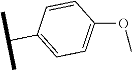 | 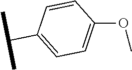 | 139% | AR4-148 |
| 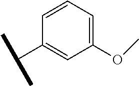 | 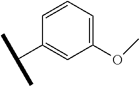 | 139% | AR4-149 |
| 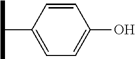 | 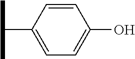 | 107% | AR5-150 |
| 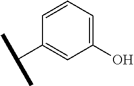 | 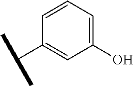 | 103% | AR4-151 |
| 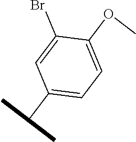 | 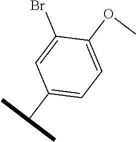 | 142% | AR4-156 |

TABLE 8
G6PD activation activity of compounds of interest including various
linkers [ a) 1 mM compound; b) 200 uM compound;
c) 100 uM compound]

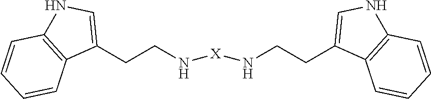

| | | Enzyme |
|---|---|---|
| Compound | X | activity (%) |
| AR3-163 | 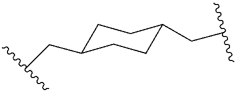 [SMILES: *CC1CCC(C*)CC1] | 137%^{a} |
| AR3-164 | 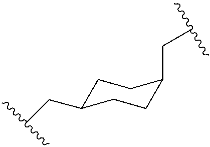 [SMILES: *CC1CCC(C*)CC1] | 89.9%^{a} |
| AR3-222 | 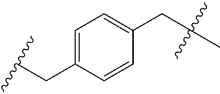 [SMILES: *Cc1ccc(C*)cc1] | 97%^{b} |
| AR3-184 | 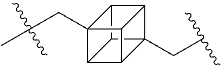 [SMILES: *CC12C3C4C1C1C2C3C41C*] | 97%^{a} |
| AR3-185 | 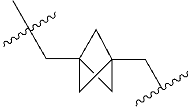 [SMILES: *CC12CC(C*)(C1)C2] | 104%^{a} |
| AR3-218 | 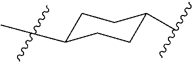 [SMILES: *C1CCC(*)CC1] | 73%^{a} |
| AR3-304 | 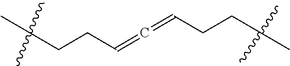 [SMILES: *CCC=C=CCC*] | 121%^{a} |
| AR4-035 | 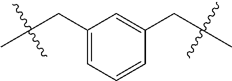 [SMILES: *Cc1cccc(C*)c1] | 126%^{c} |
| AR4-040 | 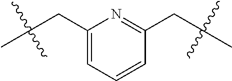 [SMILES: *Cc1cccc(C*)n1] | 108%^{c} |
| AR4-031 | 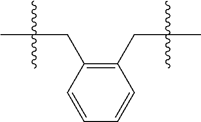 [SMILES: *Cc1ccccc1C*] | 119%^{c} |
| AR4-041 | 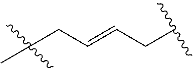 [SMILES: *C/C=C/C*] | 102%^{c} |
| AR4-033 | 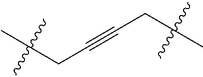 [SMILES: *CC#CC*] | 109%^{c} |
| AR4-032 | 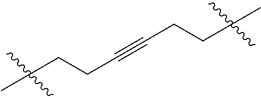 [SMILES: *CCC#CCC*] | 105%^{c} |
| AR3-069 | 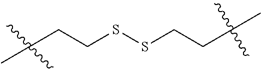 [SMILES: *CCSSCC*] | 156%^{c} |
| AR3-085 | (CH₂)₂ | 97%^{a} |
| AR3-085 | (CH₂)₃ | 60%^{a} |
| AR3-085 | (CH₂)₄ | 130%^{a} |
| AR3-085 | (CH₂)₅ | 105%^{c} |
| AR3-091 | (CH₂)₆ | 101%^{c} |
| AR3-093 | (CH₂)₇ | 125%^{c} |
| AR3-099 | (CH₂)₈ | 132%^{c} |
| AR3-107 | (CH₂)₉ | 136%^{c} |
| AR3-125 | (CH₂)₁₀ | 141%^{c} |
| AR3-140 | (CH₂)₁₂ | 151%^{c} |
| AR4-061 | 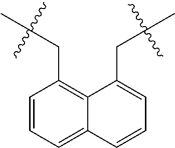 [SMILES: *Cc1cccc2cccc(C*)c12] | 127%^{c} |

TABLE 9
Activity of compound AR4-078 of interest

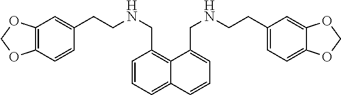 [SMILES: c1cc(CNCCc2ccc3c(c2)OCO3)c2c(CNCCc3ccc4c(c3)OCO4)cccc2c1]

| Enzyme | Canton G6PD | Canton W509A G6PD |
|---|---|---|
| Aₘₐₓ | 2.0 | 3.8 |
| AC₅₀ (uM) | 74 | 58 |

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Notwithstanding the appended claims, the disclosure set forth herein is also described by the following clauses.

Clause 1. A G6PD-modulating agent of the formula (I):

Z¹—Y—Z² (I)

wherein:

Z¹ and Z² are independently selected from an aryl, a substituted aryl, a heteroaryl, a substituted heteroaryl, a saturated carbocycle, a substituted saturated carbocycle, a heterocycle and a substituted heterocycle, wherein optionally Z¹ and Z² are each independently substituted with an amino-containing substituent comprising an amino group; and

Y is a central linking unit, optionally comprising two amino groups separated via a linker;

wherein the agent comprises at least two amino groups configured at a distance of about 4-15 angstroms,

or a salt thereof.

Clause 2. The G6PD-modulating agent of clause 1, wherein the agent is of formula (Ia):

Z¹-T¹-Y-T²-Z² (Ia)

wherein:

T¹ and T² are each independently a covalent bond or a linker; and

Y is the central linking unit and comprises two amino groups.

Clause 3. The G6PD-modulating agent of clause 1 or 2, wherein the agent is of formula (IIa):

Z¹-T¹-N(R¹)ₓ^{p+}-L¹-N(R²)_{y}^{q+}-T²-Z²;

wherein:

R¹ and R² are independently H, an alkyl, a substituted alkyl; and

L¹ is a central linker; and

x and y are independently 1 or 2, wherein:
when x is 1, p is 0;
when x is 2, p is 1;
when y is 1, q is 0; and
when y is 2, q is 1.
   Clause 4. The G6PD-modulating agent of clause 3, wherein the agent is of the formula:

Z¹-T¹-N(R)-L¹-N(R²)-T²-Z².

Clause 5. The G6PD-modulating agent of clause 4, wherein R¹ and R² are each H.
Clause 6. The G6PD-modulating agent of any one of clauses 3-5, wherein L¹ is a linker having a backbone that is 2-20 atoms in length.
Clause 7. The G6PD-modulating agent of clause 1, wherein the agent is of formula (Ib):

N(R¹)ₓ^{p+}-T¹-Z¹-L²-Z²-T²-N(R²)_{y}^{q+} (Ib)

wherein:

T¹ and T² are each independently a covalent bond or a linker;

R¹ and R² are independently H, an alkyl, a substituted alkyl; and

L² is a central linker; and

x and y are independently 2 or 3, wherein:
when x is 2, p is 0;
when x is 3, p is 1;
when y is 2, q is 0; and
when y is 3, q is 1.
   Clause 8. The G6PD-modulating agent of clause 7, wherein the agent is of the formula:

N(R¹)₂-T¹-Z¹-L²-Z²-T²-N(R²)₂.

Clause 9. The G6PD-modulating agent of clause 8, wherein R¹ and R² are each H.
Clause 10. The G6PD-modulating agent of any one of clauses 7-9, wherein L² is a linker having a backbone that is 2-12 atoms in length.
Clause 11. The G6PD-modulating agent of clause 1 or 2, wherein the agent is of formula (IIb):

Z¹-T¹-(NHetN)-T²-Z² (IIb)

wherein:

—(NHetN)— is a bivalent heterocyclic linking ring system having 1 to 4 rings and comprising a first tertiary amino group connected to T¹ and a second tertiary amino group connected to T².

Clause 12. The G6PD-modulating agent of clause 11, wherein —(NHetN)— is selected from one of the following structures:

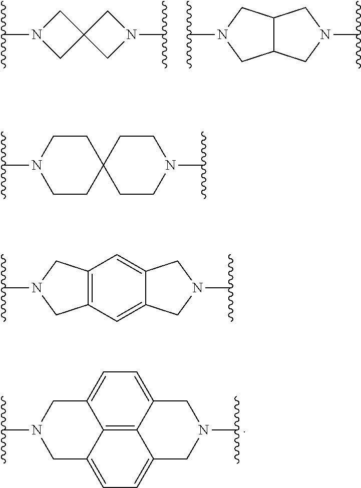

Clause 13. The G6PD-modulating agent of clause 6, wherein L¹ is of the formula:

-L¹¹-Z³-L¹²-

wherein:

L¹¹ and L¹² are independently alkyl, substituted alkyl or a polyethylene glycol (PEG) moiety; and

Z³ is selected from a covalent bond, a cycloalkyl, an aryl, a heteroaryl, a bicyclic carbocycle, a cubane, an alkenyl, an allenyl, an alkynyl and a cleavable group.

Clause 14. The G6PD-modulating agent of clause 13, wherein L¹ is selected from:

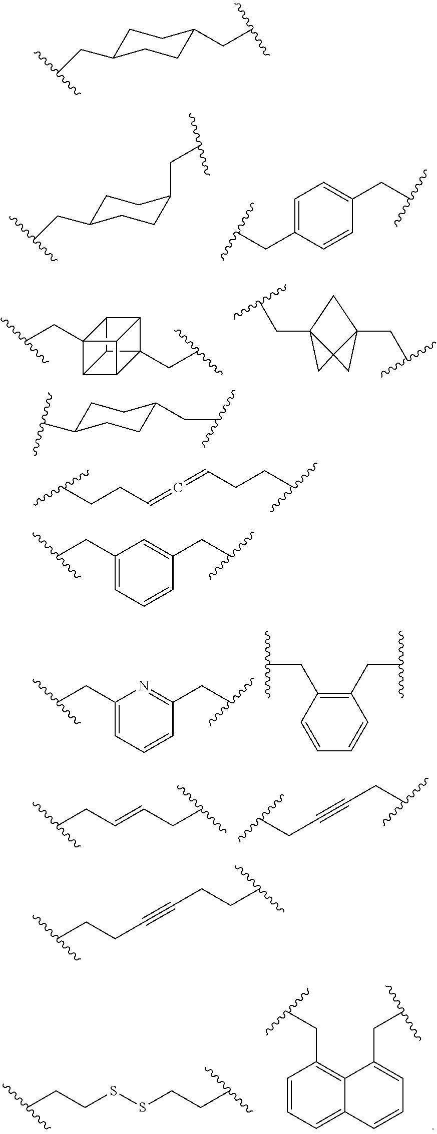

Clause 15. The G6PD-modulating agent of clause 6 wherein L is (CH₂) wherein n is 2-12.
Clause 16. The G6PD-modulating agent of clause 10, wherein L is selected from 4,4′-biphenyl, ethynylene-1,4-phenylene-ethynylene and 1,4-phenylene-ethynylene-1,4-phenylene.
Clause 17. The G6PD-modulating agent of any one of clauses 2-16, wherein T¹ and T² are independently lower alkyl or substituted lower alkyl.
Clause 18. The G6PD-modulating agent of any one of clauses 1-17, wherein Z¹ and Z² are the same.
Clause 19. The G6PD-modulating agent of any one of clauses 1-17, wherein Z¹ and Z² are different.
Clause 20. The G6PD-modulating agent of any once of clauses 1-19, wherein Z¹ and Z² are independently selected from indole, substituted indole, benzofuran, substituted benzofuran, benzothiophene, substituted benzothiophene, phenyl, substituted phenyl, quinoline, substituted quinoline, 1,3-benzodioxole, substituted 1,3-benzodioxole, thiophene, substituted thiophene, 2,3-dihydro-1H-indene, substituted 2,3-dihydro-1H-indene, pyridyl and substituted pyridyl.
Clause 21. The G6PD-modulating agent of any once of clauses 1-20, wherein Z¹ and Z² are independently selected from one of the following: 4-pyridyl, substituted 4-pyridyl, 3-pyridyl, substituted 3-pyridyl, 3-pyridyl, substituted 3-pyridyl, 2-thiophenyl, substituted 2-thiophenyl,

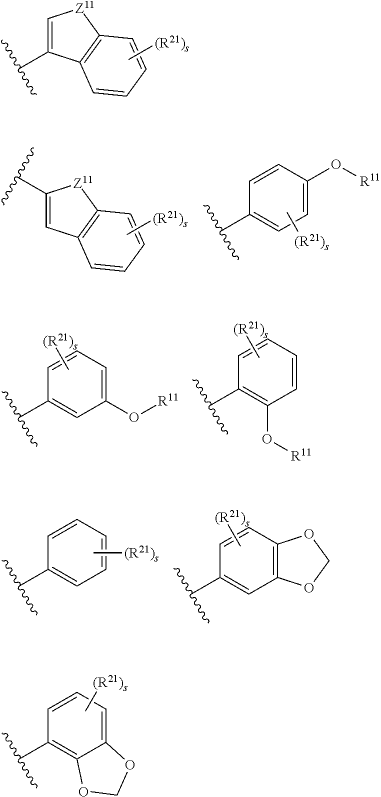

wherein:

Z¹¹ is O, S or NR, wherein R is H, alkyl or substituted alkyl;

s is 0-4;

each R²¹ is independently alkyl, substituted alkyl, halogen, hydroxy, alkoxy, substituted alkoxy, cyano, nitro, formyl (—CHO), sulfonic acid, carboxylic acid, sulfonamide or carobxyamide; and

R¹¹ is hydrogen, alkyl or substituted alkyl.

Clause 21. The G6PD-modulating agent of any once of clauses 7-10, wherein Z¹ and Z² are independently selected from the following:

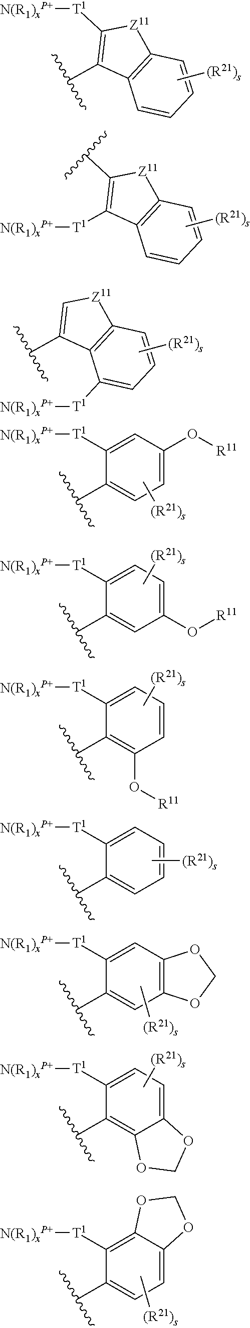

wherein:

Z¹¹ is O, S or NR, wherein R is H, alkyl or substituted alkyl;

s is 0-4 (e.g., 0, 1 or 2);

each R²¹ is independently alkyl, substituted alkyl, halogen (e.g., chloro, bromo or fluoro), hydroxy, alkoxy, substituted alkoxy, cyano, nitro, formyl (—CHO), sulfonic acid, carboxylic acid, sulfonamide or carobxyamide; and

R¹¹ is hydrogen, alkyl or substituted alkyl. In some cases of Z¹ and Z², x is 2 and p is 0.

Clause 22. The G6PD-modulating agent of clause 20, wherein T¹ is a lower alkyl or a substituted lower alkyl.
Clause 23. The G6PD-modulating agent of any one of clauses 1-22, wherein the agent is a compound of one of Tables 1-4.
Clause 24. An erythrocyte preservative composition, comprising a G6PD-modulating agent of any one of clauses 1-23.
Clause 25. A pharmaceutical composition, comprising a G6PD-modulating agent of any one of clauses 1-23 and a pharmaceutically acceptable excipient.
Clause 26. A pharmaceutical composition for use in treating a disease or condition associated with G6PD deficiency, the compositions comprising a G6PD-modulating agent of any one of clauses 1-23, and a pharmaceutically acceptable excipient.
Clause 27. A method for modulating a glucose-6-phosphate dehydrogenase (G6PD) in a sample, the method comprising:

contacting a sample comprising a G6PD with a G6PD-modulating agent of any one of clauses 1-23 to modulate the activity of the G6PD in the sample.

Clause 28. The method of clause 27, wherein the G6PD is a mutant G6PD.
Clause 29. The method of clause 28, wherein the mutant G6PD is a Canton G6PD mutant.
Clause 30. The method of clause 29, wherein the Canton G6PD mutant is the Canton single variant (R459L).
Clause 31. The method of any one of clauses 27-30, further comprising assessing the level of activity of the G6PD in the sample.
Clause 32. The method of any one of clauses 27-31, wherein the agent structurally stabilizes the G6PD to enhance catalytic activity of the G6PD.
Clause 33. The method of any one of clauses 27-32, wherein the agent activates the G6PD to a level of 110% or more.
Clause 34. The method of any one of clauses 27-33, wherein the sample is a cellular sample.
Clause 35. The method of clause 34, wherein the cellular sample comprises erythrocyte cells and the agent decreases hemolysis.
Clause 36. The method of clause 34 or 35, wherein the agent maintains or increases levels of glutathione in a cell.
Clause 37. The method of clause 35, wherein the agent increases the storage stability of a sample of erythrocyte cells.
Clause 38. The method of any one of clauses 27-37, wherein the sample is in vitro.
Clause 39. The method of any one of clauses 27-36, wherein the sample is in vivo.
Clause 40. A method for treating a subject for a G6PD deficiency-associated condition, the method comprising: administering to a subject in need thereof an effective amount of a G6PD-modulating agent according to any one of clauses 1-23 to activate a mutant G6PD and treat the subject for the G6PD deficiency-associated condition.
Clause 41. The method of clause 40, wherein the subject has a Class I-III G6PD deficiency.
Clause 42. The method of any one of clauses 40-41, wherein the condition is selected from an oxidative stress-associated condition, chronic non-spherocytic hemolytic anemia, intermittent hemolytic episode, a neurological condition, edema, kidney injury and cataract.
Clause 43. The method of any one of clauses 40-42 wherein the condition is selected from bilirubin-induced neurological injury and bilirubin encephalopathy (kernicterus).
Clause 44. The method of any one of clauses 40-43, where the subject is undergoing treatment with a drug that precipitates hemolysis in G6PD-deficient individuals.
Clause 45. Use of a pharmaceutical composition of clause 25 for the manufacture of a medicament for treating or preventing G6PD deficiency-associated condition.
Clause 46. Use of a G6PD-modulating agent of any one of clauses 1-23 for the manufacture of a medicament for treating or preventing a G6PD deficiency-associated condition.
Clause 47. The use of any one of clauses 45-46, wherein the condition is associated with a G6PD Class I-III deficiency.
Clause 48. The use of any one of clauses 45-47, wherein the condition is selected from an oxidative stress-associated condition, chronic non-spherocytic hemolytic anemia, intermittent hemolytic episode, a neurological condition, edema, kidney injury and cataract.
Clause 49. The method of any one of clauses 45-48, wherein the condition is selected from bilirubin-induced neurological injury and bilirubin encephalopathy (kernicterus).
Clause 51. A G6PD-modulating agent of the formula:

Z¹-T¹-N(R¹)ₓ^{p+}-L-N(R²)_{y}^{q+}-T²-Z²;

wherein:

Z¹ and Z² are independently selected from an aryl, a substituted aryl, a heteroaryl, a substituted heteroaryl, a saturated carbocycle, a substituted saturated carbocycle, a heterocycle and a substituted heterocycle;

T¹ and T² are each independently a covalent bond or a tether;

R¹ and R² are independently H, an alkyl, a substituted alkyl; and

L is a central linker; and

x and y are independently 1 or 2, wherein:
when x is 1, p is 0;
when x is 2, p is 1;
when y is 1, q is 0; and
when y is 2, q is 1.
   Clause 52. The G6PD-modulating agent of clause 51, wherein x and y are each 1.
   Clause 53. The G6PD-modulating agent of clause 51, wherein x and y are each 2.
   Clause 54. The G6PD-modulating agent of any one of clauses 51-53, wherein R¹ and R² are each independently H.
   Clause 55. The G6PD-modulating agent of any one of clauses 51-54, wherein L is a linker having a backbone that is 2-20 atoms in length.
   Clause 56. The G6PD-modulating agent of clause 55, wherein L is of the formula:

-L¹-Z³-L²-

wherein:

L¹ and L² are independently alkyl, substituted alkyl or a polyethylene glycol (PEG) moiety; and

Z³ is selected from a covalent bond, a cycloalkyl, an aryl, a heteroaryl, a bicyclic carbocycle, a cubane, an alkenyl, an allenyl, an alkynyl and a cleavable group.

Clause 57. The G6PD-modulating agent of clause 56, wherein L is selected from:

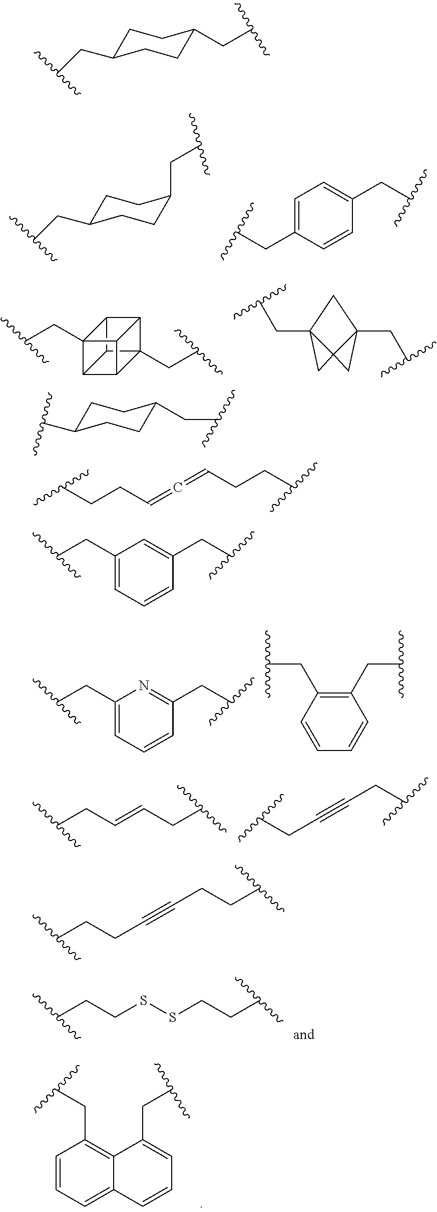

Clause 58. The G6PD-modulating agent of clause 56, wherein L is —(CH₂)ₙ— wherein n is 2-12.
Clause 59. The G6PD-modulating agent of any one of clauses 51-58, wherein Z¹ and Z² are the same.
Clause 60. The G6PD-modulating agent of any one of clauses 51-58, wherein Z¹ and Z² are different.
Clause 61. The G6PD-modulating agent of any once of clauses 51-60, wherein Z¹ and Z² are independently selected from an indole, a substituted indole, a benzofuran, a substituted benzofuran, a phenyl, a substituted phenyl, a quinoline, a substituted quinoline and a 1,3-benzodioxole a substituted 1,3-benzodioxole.
Clause 62. The G6PD-modulating agent of any one of clauses 51-61, wherein Z and Z² are independently selected from:

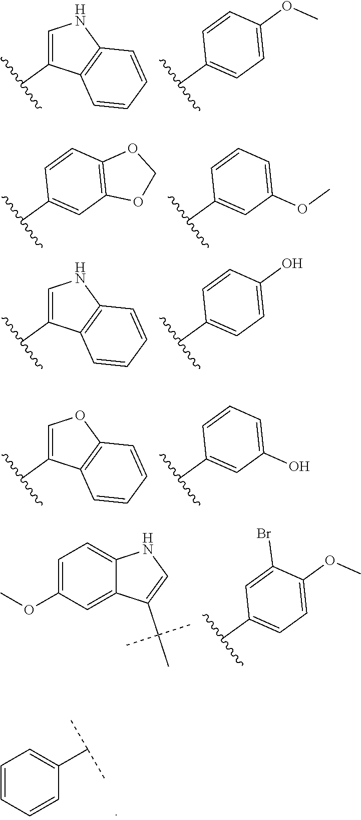

Clause 63. The G6PD-modulating agent of any one of clauses 51-62, wherein T¹ and T² are each independently a lower alkyl or a substituted lower alkyl.
Clause 64. The G6PD-modulating agent of any one of clauses 51-63, wherein Z¹-T¹- and Z²-T²- are each selected from:

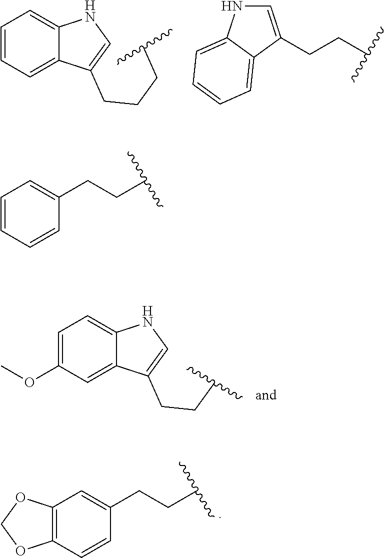

Clause 65. A method for modulating a glucose-6-phosphate dehydrogenase (G6PD) in a sample, the method comprising:

contacting a sample comprising a G6PD with a G6PD-modulating agent to activate the G6PD and/or stabilize the G6PD.

Clause 66. The method of clause 65, wherein the G6PD-modulating agent is dimeric and comprises two carbocyclic or heterocyclic groups connected via a diamino-containing linker.
Clause 67. The method of clause 66, wherein the G6PD-modulating agent is of any one of claims 51-64.
Clause 68. A method for treating a subject for a G6PD deficiency-associated condition, the method comprising:

administering to a subject in need thereof an effective amount of a G6PD-modulating agent to activate a mutant G6PD and treat the subject for the G6PD deficiency-associated condition.

Clause 69. The method of clause 68, wherein the G6PD-modulating agent is of any one of clauses 51-64.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A G6PD-modulating agent of the formula (I):
Z¹—Y—Z² (I)
wherein:
Z¹ and Z² are independently selected from an aryl, a substituted aryl, a heteroaryl, a substituted heteroaryl, a carbocycle, a substituted carbocycle, a heterocycle and a substituted heterocycle, wherein optionally Z¹ and Z² are each independently substituted with an amino-containing substituent comprising an amino group; and
Y is a central linking unit, optionally comprising two amino groups separated via a linker;
wherein the agent comprises at least two amino groups configured at a distance of about 4-15 angstroms,
or a salt thereof.

2. The G6PD-modulating agent of claim 1, wherein the agent is of formula (Ia):
Z¹-T¹-Y-T²-Z² (Ia)
wherein:
T¹ and T² are each independently a covalent bond or a linker; and
Y is the central linking unit and comprises two amino groups.

3. The G6PD-modulating agent of claim 1, wherein the agent is of formula (IIa):
Z¹-T¹-N(R¹)ₓ^{p+}-L¹-N(R²)_{y}^{q+}-T²-Z²;
wherein:
R¹ and R² are independently H, an alkyl, a substituted alkyl; and
L¹ is a central linker; and
x and y are independently 1 or 2, wherein:
when x is 1, p is 0;
when x is 2, p is 1;
when y is 1, q is 0; and
when y is 2, q is 1.

4. The G6PD-modulating agent of claim 3, wherein the agent is of the formula:
Z¹-T¹-N(R¹)-L¹-N(R²)-T²-Z².

5. The G6PD-modulating agent of claim 1, wherein the agent is of formula (Ib):
N(R¹)ₓ^{p+}-T¹-Z¹-L²-Z²-T²-N(R²)_{y}^{q+} (Ib)
wherein:
T¹ and T² are each independently a covalent bond or a linker;
R¹ and R² are independently H, an alkyl, a substituted alkyl; and
L² is a central linker; and
x and y are independently 2 or 3, wherein:
when x is 2, p is 0;
when x is 3, p is 1;
when y is 2, q is 0; and
when y is 3, q is 1.

6. The G6PD-modulating agent of claim 5, wherein the agent is of the formula:
N(R¹)₂-T¹-Z¹-L²-Z²-T²-N(R²)₂.

7. The G6PD-modulating agent of claim 1, wherein the agent is of formula (IIb):
Z¹-T¹-(NHetN)-T²-Z² (IIb)
wherein:
—(NHetN)— is a bivalent heterocyclic linking ring system having 1 to 4 rings and comprising a first tertiary amino group connected to T¹ and a second tertiary amino group connected to T².

8. The G6PD-modulating agent of claim 7, wherein —(NHetN)— is selected from one of the following structures:

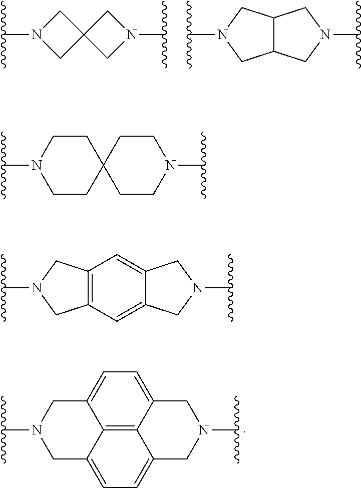

9. The G6PD-modulating agent of claim 6, wherein L¹ is of the formula:
-L¹¹-Z³-L¹²-
wherein:
L¹¹ and L¹² are independently alkyl, substituted alkyl or a polyethylene glycol (PEG) moiety; and
Z³ is selected from a covalent bond, a cycloalkyl, an aryl, a heteroaryl, a bicyclic carbocycle, a cubane, an alkenyl, an allenyl, an alkynyl and a cleavable group.

10. The G6PD-modulating agent of claim 3, wherein L¹ is —(CH₂)ₙ— wherein n is 2-12.

11. The G6PD-modulating agent of claim 5, wherein L² is selected from 4,4′-biphenyl, ethynylene-1,4-phenylene-ethynylene and 1,4-phenylene-ethynylene-1,4-phenylene.

12. The G6PD-modulating agent of claim 1, wherein Z¹ and Z² are independently selected from indole, substituted indole, benzofuran, substituted benzofuran, benzothiophene, substituted benzothiophene, phenyl, substituted phenyl, quinoline, substituted quinoline, 1,3-benzodioxole, substituted 1,3-benzodioxole, thiophene, substituted thiophene, 2,3-dihydro-1H-indene, substituted 2,3-dihydro-1H-indene, pyridyl and substituted pyridyl.

13. The G6PD-modulating agent of claim 1, wherein Z¹ and Z² are independently selected from one of the following:
4-pyridyl, substituted 4-pyridyl, 3-pyridyl, substituted 3-pyridyl, 3-pyridyl, substituted 3-pyridyl, 2-thiophenyl, substituted 2-thiophenyl, 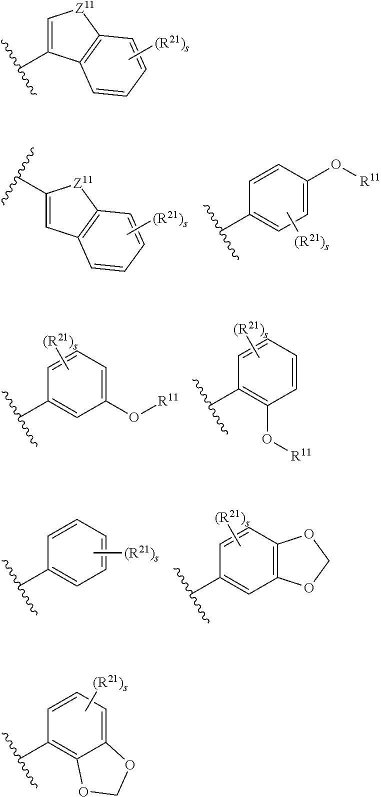
wherein:
Z¹¹ is O, S or NR, wherein R is H, alkyl or substituted alkyl;
s is 0-4;
each R²¹ is independently alkyl, substituted alkyl, halogen, hydroxy, alkoxy, substituted alkoxy, cyano, nitro, formyl (—CHO), sulfonic acid, carboxylic acid, sulfonamide or carobxyamide; and
R¹¹ is hydrogen, alkyl or substituted alkyl.

14. The G6PD-modulating agent of claim 5, wherein Z¹ and Z² are independently selected from the following: 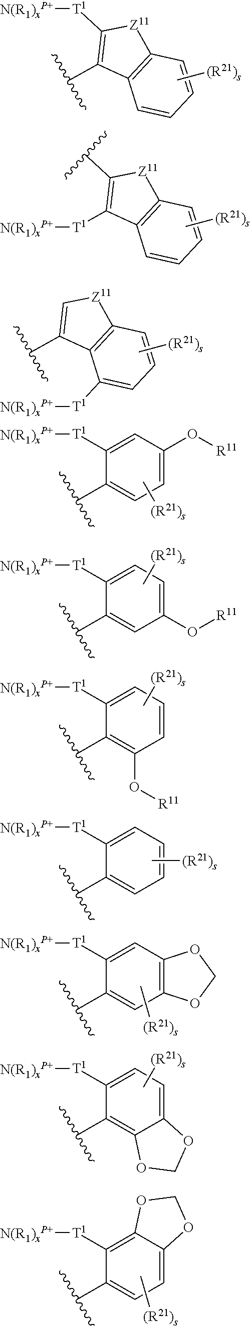
wherein:
Z¹¹ is O, S or NR, wherein R is H, alkyl or substituted alkyl;
s is 0-4 (e.g., 0, 1 or 2);
each R²¹ is independently alkyl, substituted alkyl, halogen (e.g., chloro, bromo or fluoro), hydroxy, alkoxy, substituted alkoxy, cyano, nitro, formyl (—CHO), sulfonic acid, carboxylic acid, sulfonamide or carobxyamide; and
R¹¹ is hydrogen, alkyl or substituted alkyl. In some cases of Z¹ and Z², x is 2 and p is 0.

15. An erythrocyte preservative composition, comprising a G6PD-modulating agent of claim 1.

16. A pharmaceutical composition, comprising a G6PD-modulating agent of claim 1 and a pharmaceutically acceptable excipient.

17. A method for modulating a glucose-6-phosphate dehydrogenase (G6PD) in a sample, the method comprising:
contacting a sample comprising a G6PD with a G6PD-modulating agent of claim 1 to modulate the activity of the G6PD in the sample.

18. The method of claim 17, wherein the agent increases the storage stability of a sample of erythrocyte cells.

19. A method for treating a subject for a G6PD deficiency-associated condition, the method comprising:
administering to a subject in need thereof an effective amount of a G6PD-modulating agent according to claim 1 to activate a mutant G6PD and treat the subject for the G6PD deficiency-associated condition.

20. The method of claim 19, wherein the subject is undergoing treatment with a drug that precipitates hemolysis in G6PD-deficient individuals.
